SEQUENCE LISTING (23 sequences)

SEQ ID NO: 1 - protein, 706 aa, Artificial Sequence
  BCL-6 polypeptide
MASPADSCIQFTRHASDVLLNLNRLRSRDILTDVVIVVSREQFRAHKTVLMACSGLFYSIFTDQLKCNLSVINLDPEINPEGFCILLDFMYTSRLNLREGNIMAVMATAMYLQMEHVVDTCRKFIKASEAEMVSAIKPPREEFLNSRMLMPQDIMAYRGREVVENNLPLRSAPGCESRAFAPSLYSGLSTPPASYSMYSHLPVSSLLFSDEEFRDVRMPVANPFPKERALPCDSARPVPGEYSRPTLEVSPNVCHSNIYSPKETIPEEARSDMHYSVAEGLKPAAPSARNAPYFPCDKASKEEERPSSEDEIALHFEPPNAPLNRKGLVSPQSPQKSDCQPNSPTESCSSKNACILQASGSPPAKSPTDPKACNWKKYKFIVLNSLNQNAKPEGPEQAELGRLSPRAYTAPPACQPPMEPENLDLQSPTKLSASGEDSTIPQASRLNNIVNRSMTGSPRSSSESHSPLYMHPPKCTSCGSQSPQHAEMCLHTAGPTFPEEMGETQSEYSDSSCENGAFFCNECDCRFSEEASLKRHTLQTHSDKPYKCDRCQASFRYKGNLASHKTVHTGEKPYRCNICGAQFNRPANLKTHTRIHSGEKPYKCETCGARFVQVAHLRAHVLIHTGEKPYPCEICGTRFRHLQTLKSHLRIHTGEKPYHCEKCNLHFRHKSQLRLHLRQKHGAITNTKVQYRVSATDLPPELPKAC

SEQ ID NO: 2 - protein, 20 aa, Artificial Sequence
  Anti-BCL-6 Antibody Epitope
TKVQYRVSATDLPPELPKAC

SEQ ID NO: 3 - protein, 112 aa, Artificial Sequence
  Anti-BCL-6 Antibody Heavy Chain Variable Region (VH)
QSLEESGGDLVKPGASLTLTCTASGFSFNKDAVCWVRQAPGKGLEWIACIYAGSSGSTYYATWAKGRFTISETSSTTVTLRMTSLTAADTATYFCSRGTNVWGPGTLVTVSS

SEQ ID NO: 4 - protein, 113 aa, Artificial Sequence
  Anti-BCL-6 Antibody Light Chain Variable Region (VL)
ADIAMTQTPSSVEAAVGGTVTIKCQASQTINSYLAWYQQKPGQPPKLLIYRASTLASGAPSRFKGSGSGTQFTLTISDLECADAATYYCQSYYYSSNTAYGNTFGGGTDVVVK

SEQ ID NO: 5 - protein, 7 aa, Artificial Sequence
  Anti-BCL-6 Antibody Heavy Chain CDR1
GFSFNKD

SEQ ID NO: 6 - protein, 7 aa, Artificial Sequence
  Anti-BCL-6 Antibody Heavy Chain CDR2
YAGSSGS

SEQ ID NO: 7 - protein, 4 aa, Artificial Sequence
  Anti-BCL-6 Antibody Heavy Chain CDR3
GTNV

SEQ ID NO: 8 - protein, 24 aa, Artificial Sequence
  Anti-BCL-6 Antibody Heavy Chain FR1
QSLEESGGDLVKPGASLTLTCTAS

SEQ ID NO: 9 - protein, 19 aa, Artificial Sequence
  Anti-BCL-6 Antibody Heavy Chain FR2
AVCWVRQAPGKGLEWIACI

SEQ ID NO: 10 - protein, 40 aa, Artificial Sequence
  Anti-BCL-6 Antibody Heavy Chain FR3
TYYATWAKGRFTISETSSTTVTLRMTSLTAADTATYFCSR

SEQ ID NO: 11 - protein, 11 aa, Artificial Sequence
  Anti-BCL-6 Antibody Heavy Chain FR4
WGPGTLVTVSS

SEQ ID NO: 12 - protein, 11 aa, Artificial Sequence
  Anti-BCL-6 Antibody Light Chain CDR1
QASQTINSYLA

SEQ ID NO: 13 - protein, 7 aa, Artificial Sequence
  Anti-BCL-6 Antibody Light Chain CDR2
RASTLAS

SEQ ID NO: 14 - protein, 14 aa, Artificial Sequence
  Anti-BCL-6 Antibody Light Chain CDR3
QSYYYSSNTAYGNT

SEQ ID NO: 15 - protein, 23 aa, Artificial Sequence
  Anti-BCL-6 Antibody Light Chain FR1
DIAMTQTPSSVEAAVGGTVTIKC

SEQ ID NO: 16 - protein, 15 aa, Artificial Sequence
  Anti-BCL-6 Antibody Light Chain FR2
WYQQKPGQPPKLLIY

SEQ ID NO: 17 - protein, 32 aa, Artificial Sequence
  Anti-BCL-6 Antibody Light Chain FR3
GAPSRFKGSGSGTQFTLTISDLECADAATYYC

SEQ ID NO: 18 - protein, 10 aa, Artificial Sequence
  Anti-BCL-6 Antibody Light Chain FR4
FGGGTDVVVK

SEQ ID NO: 19 - protein, 323 aa, Artificial Sequence
  Anti-BCL-6 Antibody Heavy Chain Constant Region
GQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 20 - protein, 104 aa, Artificial Sequence
  Anti-BCL-6 Antibody Light Chain Constant Region
GDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 21 - protein, 435 aa, Artificial Sequence
  Anti-BCL-6 Antibody Full Heavy Chain
QSLEESGGDLVKPGASLTLTCTASGFSFNKDAVCWVRQAPGKGLEWIACIYAGSSGSTYYATWAKGRFTISETSSTTVTLRMTSLTAADTATYFCSRGTNVWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 22 - protein, 217 aa, Artificial Sequence
  Anti-BCL-6 Antibody Full Light Chain
ADIAMTQTPSSVEAAVGGTVTIKCQASQTINSYLAWYQQKPGQPPKLLIYRASTLASGAPSRFKGSGSGTQFTLTISDLECADAATYYCQSYYYSSNTAYGNTFGGGTDVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 23 - protein, 24 aa, Artificial Sequence
  Anti-BCL-6 Antibody Light Chain FR1
ADIAMTQTPSSVEAAVGGTVTIKC

(19) United States
(12) Patent Application Publication
(10) Pub. No.: US 2024/0294633 A1
(43) Pub. Date: Sep. 5, 2024

(54) ANTIBODY SPECIFIC FOR ANTI-BCL-6 ANTIBODY AND METHODS OF USE

(71) Applicant: NONAGEN THERAPEUTICS CORPORATION, Los Angeles, CA (US)

(72) Inventors: Charles J. ROSSER, Stevenson Ranch, CA (US); Hideki FURUYA, Beverly Hills, CA (US)

(21) Appl. No.: 18/566,522

(22) PCT Filed: Jun. 6, 2022
(86) PCT No.: PCT/US2022/032373

§ 371 Date: Dec. 1, 2023

Related U.S. Application Data
(60) Provisional application No. 63/197,112, filed on Jun. 4, 2021.

(51) Int. Cl. C07K 16/28; A61K 39/00; A61P 35/00; G01N 33/543

(52) U.S. Cl. CPC C07K 16/28; A61P 35/00; G01N 33/543; A61K 2039/505; C07K 2317/24; C07K 2317/565; C07K 2317/567

(57) ABSTRACT

Antibodies against human B cell lymphoma 6 protein (BCL-6) are provided. The antibodies have neutralizing activity against BCL-6. The anti-human B cell lymphoma 6 protein antibodies may be used in therapeutic treatment in patients with angiogenesis-dependent conditions.

### CROSS REFERENCE TO RELATED APPLICATIONS

This application is the National Stage entry of International Application No. PCT/US22/32373, filed 6 Jun. 2022, which claims priority to U.S. No. 63/197,112, filed 4 Jun. 2021, and entitled ANTIBODY SPECIFIC FOR ANTI-BCL-6 ANTIBODY AND METHODS OF USE. The content of the aforementioned application is herein incorporated by reference in its entirety.

### REFERENCE TO SEQUENCE LISTING SUBMITTED ELECTRONICALLY

Pursuant to the EFS-Web legal framework and 37 CFR §§ 1.821-825 (see MPEP § 2442.03(a)), a Sequence Listing in the form of an ASCII-compliant text file (entitled “3000047-003977_Sequence_Listing_ST25” created on Jun. 6, 2022 and 22,779 bytes in size) is submitted concurrently with the instant application, and the entire contents of the Sequence Listing are incorporated herein by reference.

### BACKGROUND

### 1. Field

The present disclosure relates to antibodies that specifically bind the human B cell lymphoma 6 protein (BCL-6). The antibodies have neutralizing activity against human B cell lymphoma 6 protein (BCL-6) and its biological function and can be used as a therapeutic in patients suffering from cancer, and other proliferative diseases, and complications thereof.

### 2. Description of Related Art

Angiogenesis is a process involving the growth of new blood vessels from pre-existing vessels. In healthy adults, angiogenesis is quiescent in most of the organs; however, angiogenesis may occur wound healing and in certain pathological conditions, such as during benign or malignant tumor growth or in retinal disorders. Several therapeutic compounds, including humanized monoclonal antibodies, target angiogenesis, an essential aspect of tumor development. For example, AVASTIN® (bevacizumab), a humanized monoclonal antibody, that targets the angiogenic factor VEGF (Vascular Endothelial Growth Factor) has obtained Food and Drug Agency (FDA) approval for treating cancers in association with standard chemotherapeutic agents. Hurwitz Clin Colorectal Cancer (2004) 2 Suppl 2:S62-8, incorporated herein by reference in its entirety. However, targeting VEGF alone has been only partially successful. See, e.g., Yang et al. Angiogenesis (2003) 6(2): 151-8; Escudier et al. Expert Rev Anticancer Ther. (2008) 8(10): 1545-57; Escudier et al. Drugs (2011) 71(9): 1179-91, incorporated herein by reference in its entirety. Evasion of treatment may arise via various mechanisms so there remains a need in the art for additional anti-angiogenic compounds that can be used alone or in combination with anti-cancer treatments to improve clinical management and outcomes for cancer patients.

### BRIEF SUMMARY

The present invention encompasses antibodies (including antigen-binding molecules comprising, or alternatively consisting of, antibody fragments or variants thereof) that immunospecifically bind to human B cell lymphoma 6 protein (BCL-6), and compositions, and methods of use thereof. In particular, the invention provides antibodies (including antigen-binding molecules comprising, or alternatively consisting of, antibody fragments or variants thereof) that immunospecifically bind to human B cell lymphoma 6 protein (BCL-6) (SEQ ID NO: 1), preferably an epitope comprising (SEQ ID NO: 2). The present invention further provides methods and compositions for preventing, treating or ameliorating proliferative diseases or disorders, preferably cancer, optionally, solid tumors, in an animal, preferably a mammal, and most preferably a human, comprising, or alternatively consisting of, administering to said animal an effective amount of one or more antibodies (including antigen-binding molecules comprising, or alternatively consisting of, antibody fragments or variants thereof) that immunospecifically bind to BCL-6. Diseases and disorders which can be prevented, treated or ameliorated by administering an effective amount of an antibody described herein include, but are not limited to, cancer, optionally solid tumors.

The present inventors identified antibodies that immunospecifically bind to BCL-6, in particular, to the BCL-6, including an epitope comprising the amino acid sequence of SEQ ID NO: 2. The whole antibodies described herein may have a general “Y” structure consisting of four polypeptides, two heavy chains and two light chains, wherein each heavy chain and each light chain may be identical. Each heavy and light chain may consist of a constant region (or constant domain) and a variable region (or constant region), e.g., variable heavy (HV) and variable light (LH). Each heavy and light chain may comprise a set of three (3) complementarity-determining regions (CDRs) in the variable regions. Antigen-binding molecules comprising, or alternatively consisting of, fragments or variants of these antibodies (e.g., including VH domains, VH CDRs, VL domains, or VL CDRs having an amino acid sequence of any one of those described herein), that immunospecifically bind to BCL-6, are also encompassed by the invention, as are nucleic acid molecules that encode these antibodies, vectors and host cells comprising the same, and/or antigen-binding molecules. Fragments or variants may include, e.g., Fabs, F(ab′)2 fragments, Fc fragments, disulfide-linked Fvs (sdFvs), antiidiotypic (anti-Id) antibodies, and scFvs.

In one embodiment, antibodies may comprise a heavy chain constant region. Antigen-binding molecules comprising, or alternatively consisting of, fragments or variants of these monoclonal antibodies (e.g., including VH domains, VH CDRs, VL domains, or VL CDRs), that immunospecifically bind to BCL-6, also are described are nucleic acid molecules that encode these antibodies, vectors and host cells comprising the same, and/or antigen-binding molecules.

In one embodiment, antibodies may comprise a light chain constant region. Antigen-binding molecules comprising, or alternatively consisting of, fragments or variants of these monoclonal antibodies (e.g., including VH domains, VH CDRs, VL domains, or VL CDRs), that immunospecifically bind to BCL-6, also are described are nucleic acid molecules that encode these antibodies, vectors and host cells comprising the same, and/or antigen-binding molecules.

Molecules comprising, or alternatively consisting of, fragments or variants the heavy chain constant region that immunospecifically bind to BCL-6, are also encompassed by the invention, as are nucleic acid molecules that encode these antigen-binding molecules.

Molecules comprising, or alternatively consisting of, fragments or variants the light chain constant region that immunospecifically bind to BCL-6, are also encompassed by the invention, as are nucleic acid molecules that encode these antigen-binding molecules.

In one embodiment, antibodies (including molecules comprising, or alternatively consisting of, antibody fragments or variants thereof) may immunospecifically bind to a polypeptide or a polypeptide fragment of BCL-6 (SEQ ID NO: 1, 2, or combinations thereof), said antibodies comprising, or alternatively consisting of, a polypeptide having the amino acid sequence of any one, two, three or more of the VH complementarity determining regions (“CDRs”) (e.g., VH CDR1, VH CDR2, or VH CDR3) of a VH domain and/or any one, two, three or more of the VL CDRs (e.g., VL CDR1, VL CDR2, or VL CDR3) of a VL domain.

In one embodiment, antibodies (including molecules comprising, or alternatively consisting of, antibody fragments or variants thereof (including derivatives)) may comprise, or alternatively consist of, of VH domains, VL domains and/or CDRs described herein, which antibodies, immunospecifically bind to BCL-6 (e.g., the BCL-6) and can be routinely assayed for immunospecific binding to BCL-6 using methods known in the art, such as, for example, the immunoassays disclosed herein. Antibodies and antibody fragments or variants (including derivatives) described herein may include, for example, one or more amino acid sequence alterations (addition, deletion, substitution and/or insertion of an amino acid residue). These alterations may be made in one or more framework regions and/or one or more CDRs. Amino acid changes in the CDRs may preferably be conservative amino acid substitutions and/or me be preferably performed without disrupting the BCL-6 binding specificity. The antibodies described herein (including antibody fragments, and variants and derivative thereof) can be routinely made by methods known in the art. Molecules comprising, or alternatively consisting of, fragments or variants of any of the VH domains, VH CDRs, VL domains, and VL CDRs whose sequences are specifically disclosed herein may be employed in accordance with the present invention. Nucleic acid molecules encoding these antibodies and molecules (including fragments, variants, and derivatives) are also encompassed by the invention.

In one embodiment, panels of antibodies (including molecules comprising, or alternatively consisting of, antibody fragments or variants) are described, wherein the panel members correspond to one, two, three, four, five, ten, fifteen, twenty, or more different antibodies described herein (e.g., whole antibodies, Fabs, F(ab′)2 fragments, Fc fragments, disulfide-linked Fvs (sdFvs), antiidiotypic (anti-Id) antibodies, and scFvs). The present invention further provides mixtures of antibodies, wherein the mixture corresponds to one, two, three, four, five, ten, fifteen, twenty, or more different antibodies described herein (e.g., whole antibodies, Fabs, F(ab′)2 fragments, Fc fragments, disulfide-linked Fvs (sdFvs), antiidiotypic (anti-Id) antibodies, and scFvs).

In one embodiment, compositions may comprise or consist of one, two, three, four, five, ten, fifteen, twenty, or more antibodies of the present invention (including molecules comprising, or alternatively consisting of, antibody fragments or variants thereof). A composition described herein may comprise, or alternatively consist of, one, two, three, four, five, ten, fifteen, twenty, or more amino acid sequences of one or more antibodies or fragments or variants thereof. Alternatively, a composition described herein may comprise, or alternatively consist of, nucleic acid molecules encoding one or more antibodies described herein.

In one embodiment, fusion proteins may comprise an antibody (including molecules comprising, or alternatively consisting of, antibody fragments or variants thereof) described herein, and a heterologous polypeptide (e.g., a polypeptide unrelated to an antibody or antibody domain). Nucleic acid molecules encoding these fusion proteins are also encompassed by the invention. A composition of the present invention may comprise, or alternatively consist of, one, two, three, four, five, ten, fifteen, twenty or more fusion proteins described herein. Alternatively, a composition described herein may comprise, or alternatively consist of, nucleic acid molecules encoding one, two, three, four, five, ten, fifteen, twenty or more fusion proteins described herein.

In one embodiment, a recombinant nucleic acid molecule, which may be isolated, encoding an antibody (including molecules which may comprise or consist of an antibody fragment or variant thereof) described herein is provided. The present invention also provides a host cell transformed with a nucleic acid molecule described herein and progeny thereof. The present invention also provides a method for the production of an antibody (including a molecule comprising, or alternatively consisting of, an antibody fragment or variant thereof) described herein. The present invention further provides a method of expressing an antibody (including a molecule comprising, or alternatively consisting of, an antibody fragment or variant thereof) described herein from a recombinant nucleic acid molecule. These and other aspects described herein are described in further detail below.

In one embodiment, methods and compositions for treating proliferative disorders, optionally cancer, preferably a solid tumor, in an animal, preferably a mammal, and most preferably a human, may comprise using antibodies (including molecules which comprise, or alternatively consist of, antibody fragments or variants thereof) that immunospecifically bind to BCL-6. In an embodiment, the antibodies and antibody fragments described herein may be used in compositions and/or methods for the treatment of a disease characterized by excessive neovascularization. In an embodiment, the antibodies and antibody fragments described herein may be used in compositions and/or methods for the treatment of a disease characterized by pathological angiogenesis. Diseases and disorders which can be treated with the antibodies described herein include, but are not limited to abnormal angiogenesis, ophthalmological diseases with abnormal angiogenesis, rheumatoid arthritis, psoriasis, angioma, endometriosis, Kaposi sarcoma associated with the extracellular presence of human B cell lymphoma or fragments thereof, and combinations thereof.

In an embodiment, an anti-human B cell lymphoma 6 protein antibody or an antigen binding fragment thereof may comprise a heavy chain variable (VH) region and a light chain variable (VL) region that specifically binds a human B cell lymphoma 6 protein (BCL-6) polypeptide.

In an embodiment, a recombinant and/or isolated antibody or antigen binding fragment thereof comprising: (a) a heavy chain variable domain comprising (i) a complementarity-determining region (CDR) 1 comprising the amino acid sequence of SEQ ID NO: 5 comprising at most one conservative substitution, (ii) a CDR 2 comprising the amino acid sequence of SEQ ID NO: 6 comprising at most one conservative substitution, and (iii) a CDR 3 comprising the amino acid sequence of SEQ ID NO: 7 comprising at most one conservative substitution; and (b) a light chain variable domain comprising (i) a CDR 1 comprising the amino acid sequence of SEQ ID NO: 12 comprising at most one conservative substitution, (ii) a CDR 2 comprising the amino acid sequence of SEQ ID NO: 13 comprising at most one conservative substitution, and (iii) a CDR 3 comprising the amino acid sequence of SEQ ID NO: 14 comprising at most one conservative substitution may be provided.

In an embodiment, (a) the heavy chain variable domain may comprise (i) a CDR 1 comprising the amino acid sequence of SEQ ID NO: 5, (ii) a CDR 2 comprising the amino acid sequence of SEQ ID NO: 6, and (iii) a CDR 3 comprising the amino acid sequence of SEQ ID NO: 7; and (b) the light chain variable domain may comprise (i) a CDR 1 comprising the amino acid sequence of SEQ ID NO: 12, (ii) a CDR 2 comprising the amino acid sequence of SEQ ID NO: 13, and (iii) a CDR 3 comprising the amino acid sequence of SEQ ID NO: 14.

In an embodiment, (a) the heavy chain variable domain may comprise (i) a framework region (FR) 1 comprising an amino acid sequence at least about 95% identical to SEQ ID NO: 8, (ii) a FR 2 comprising an amino acid sequence at least about 95% identical to SEQ ID NO: 9, (iii) a FR 3 comprising an amino acid sequence at least about 95% identical to SEQ ID NO: 10, (iv) a FR 4 comprising an amino acid sequence at least about 95% identical to SEQ ID NO: 11; and (b) the light chain variable domain may comprise (i) a FR 1 comprising an amino acid sequence at least about 95% identical to SEQ ID NO: 15, or a FR 1 comprising an amino acid sequence at least about 95% identical to SEQ ID NO: 23, (ii) a FR 2 comprising an amino acid sequence at least about 95% identical to SEQ ID NO: 16, (iii) a FR 3 comprising an amino acid sequence at least about 95% identical to SEQ ID NO: 17, (iv) a FR 4 comprising an amino acid sequence at least about 95% identical to SEQ ID NO: 18.

In an embodiment, (a) the heavy chain variable domain may comprise (i) a framework region (FR) 1 comprising the amino acid sequence of SEQ ID NO: 8, (ii) a FR 2 comprising the amino acid sequence of SEQ ID NO: 9, (iii) a FR 3 comprising the amino acid sequence of SEQ ID NO: 10, and (iv) a FR 4 comprising the amino acid sequence of SEQ ID NO: 11; and (b) the light chain variable domain may comprise (i) a FR 1 comprising the amino acid sequence of SEQ ID NO: 15 or 23, (ii) a FR 2 comprising the amino acid sequence of SEQ ID NO: 16, (iii) a FR 3 comprising the amino acid sequence of SEQ ID NO: 17, (iv) a FR 4 comprising the amino acid sequence of SEQ ID NO: 18.

In an embodiment, a recombinant and/or isolated antibody or an antigen binding fragment thereof comprising: (a) a heavy chain variable domain comprising the amino acid sequence of SEQ ID NO: 3 or an amino acid sequence at least about 85%, at least about 90%, at least about 91%, at least about 92%, at least about 93%, at least about 94%, at least about 95%, at least about 96%, at least about 97%, at least about 98%, or at least about 99% identical thereto; and (b) a light chain variable domain comprising the amino acid sequence of SEQ ID NO: 4 or an amino acid sequence at least about 85%, at least about 90%, at least about 91%, at least about 92%, at least about 93%, at least about 94%, at least about 95%, at least about 96%, at least about 97%, at least about 98%, or at least about 99% identical thereto may be provided.

In an embodiment, the recombinant and/or isolated antibody or an antigen binding fragment may further comprise: (a) a heavy chain constant domain or portion thereof, (b) a light chain constant domain or portion thereof, or (c) a combination of (a) and (b).

In an embodiment, (a) the heavy chain constant domain or portion thereof may be or may be derived from an alpha (α), delta (δ), epsilon (ε), gamma (γ), or mu (μ) constant domain; (b) the light chain constant domain or portion thereof may be or may be derived from a kappa (κ) or lambda (λ) constant domain; or (c) a combination of (a) and (b).

In an embodiment, (a) the heavy chain constant domain may be or may be derived from a gamma (γ) constant domain; (b) the light chain constant domain may be or may be derived from a kappa (κ) constant domain; or (c) a combination of (a) and (b).

In an embodiment, (a) the heavy chain constant domain may comprise the amino acid sequence of SEQ ID NO: 19 or an amino acid sequence at least about 85%, at least about 90%, at least about 91%, at least about 92%, at least about 93%, at least about 94%, at least about 95%, at least about 96%, at least about 97%, at least about 98%, or at least about 99% identical thereto; and (b) the light chain constant domain may comprise the amino acid sequence of SEQ ID NO: 20 or an amino acid sequence at least about 85%, at least about 90%, at least about 91%, at least about 92%, at least about 93%, at least about 94%, at least about 95%, at least about 96%, at least about 97%, at least about 98%, or at least about 99% identical thereto; or (c) a combination of (a) and (b).

In an embodiment, the antigen binding fragment may an scFv, Fab, Fab′, or F(ab′)2.

In an embodiment, a recombinant and/or isolated antibody comprising: (i) a heavy chain comprising the amino acid sequence of SEQ ID NO: 21 or an amino acid sequence at least about 85%, at least about 90%, at least about 91%, at least about 92%, at least about 93%, at least about 94%, at least about 95%, at least about 96%, at least about 97%, at least about 98%, or at least about 99% identical thereto; and (ii) a light chain comprising the amino acid sequence of SEQ ID NO: 22 or an amino acid sequence at least about 85%, at least about 90%, at least about 91%, at least about 92%, at least about 93%, at least about 94%, at least about 95%, at least about 96%, at least about 97%, at least about 98%, or at least about 99% identical thereto may be provided.

In an embodiment, the antibody or antigen binding fragment thereof may be monoclonal. In an embodiment, the recombinant and/or isolated antibody or antigen binding fragment thereof may be humanized. In an embodiment, the antibody or antigen binding fragment thereof may be chimeric.

In an embodiment, the recombinant and/or isolated antibody or an antigen binding fragment thereof may specifically bind a human B cell lymphoma 6 protein (BCL-6) polypeptide.

In an embodiment, the antibody or antibody binding fragment thereof may specifically bind to a BCL-6 having at least about 80%, at least about 85%, at least about 90%, at least about 91%, at least about 92%, at least about 93%, at least about 94%, at least about 95%, at least about 96%, at least about 97%, at least about 98%, or at least about 99% sequence homology to an amino acid sequence comprising the amino acid sequence of SEQ ID NO: 1. In an embodiment, the antibody or antibody binding fragment thereof may specifically bind to an epitope on BCL-6 having at least about 80%, at least about 85%, at least about 90%, at least about 91%, at least about 92%, at least about 93%, at least about 94%, at least about 95%, at least about 96%, at least about 97%, at least about 98%, or at least about 99% sequence homology to an amino acid sequence comprising the amino acid sequence of SEQ ID NO: 2.

In an embodiment, the recombinant and/or isolated antibody or antigen binding fragment thereof may be conjugated to a label, cytotoxic agent, immunosuppressive agent, toxin, or a combination thereof. In an embodiment, the recombinant and/or isolated antibody or antigen binding fragment thereof may be conjugated to a detectable label. In an embodiment, the detectable label may be selected from a fluorescent label, luminescent label, bioluminescent label, radioactive label, chemiluminescent label, colorimetric label, fluorogenic label, enzymatic label, or a combination thereof.

In an embodiment, an anti-BCL-6 antibody that competes for binding to human B cell lymphoma 6 protein with a recombinant and/or isolated antibody or fragment thereof described herein may be provided.

In an embodiment, a composition comprising the recombinant and/or isolated antibody or fragment thereof described herein may be provided. In an embodiment, the composition may be a pharmaceutical composition and may further comprise a pharmaceutical excipient, carrier, diluent, adjuvant, or a combination thereof. In an embodiment, the adjuvant may be selected from the group consisting of anti-CD40 antibody, imiquimod, resiquimod, GM-CSF, cyclophosphamide, sunitinib, bevacizumab, interferon-alpha, interferon-beta, CpG oligonucleotides and derivatives, poly-(I:C) and derivatives, RNA, sildenafil, particulate formulations with poly(lactide co-glycolide) (PLG), virosomes, or an interleukin (IL).

In an embodiment, a fusion protein comprising the recombinant and/or isolated antibody or fragment thereof described herein may be provided. In an embodiment, the fusion protein may be isolated.

In an embodiment, a polynucleotide comprising a nucleic acid sequence encoding the recombinant and/or isolated antibody or fragment thereof described herein may be provided. In an embodiment, a polynucleotide comprising a nucleic acid sequence (i) encoding any one or a combination of the amino acid sequence of SEQ ID NO: 3-22, or (ii) a polynucleotide comprising a nucleic acid with about 80%. about 85%, about 90%, about 91%, about 92%, about 93%, about 94%, about 95%, about 96%, about 97% about 98%, or about 99% identity thereto may be provided. In an embodiment, a polynucleotide comprising a nucleic acid sequence encoding the fusion protein described herein may be provided. In an embodiment, polynucleotide may be isolated.

In an embodiment, a vector comprising the polynucleotide described herein may be provided. In an embodiment, the vector may be isolated.

In an embodiment, a host cell comprising the vector described herein may be provided. In an embodiment, the host cell may be isolated.

In an embodiment, a composition comprising the fusion protein described herein may be provided. In an embodiment, a composition comprising the polynucleotide described herein may be provided. In an embodiment, a composition comprising the vector described herein may be provided. In an embodiment, composition comprising the host cell described herein may be provided. In an embodiment, the composition may be a pharmaceutical composition and further comprises a pharmaceutically acceptable excipient, carrier, diluent, adjuvant, vehicle, or a combination thereof. In an embodiment, the adjuvant may be selected from the group consisting of anti-CD40 antibody, imiquimod, resiquimod, GM-CSF, cyclophosphamide, sunitinib, bevacizumab, interferon-alpha, interferon-beta, CpG oligonucleotides and derivatives, poly-(I:C) and derivatives, RNA, sildenafil, particulate formulations with poly(lactide co-glycolide) (PLG), virosomes, or an interleukin (TL).

In an embodiment, a method for detecting a human B cell lymphoma 6 protein polypeptide comprising contacting a sample with the isolated and/or recombinant antibody or antigen binding fragment thereof described herein may be provided. In an embodiment, the antibody or antigen binding fragment thereof, may be attached to a solid phase support. In an embodiment, the solid phase support may be an array, a bead, plate, matrix, polymer, test tube, sheet, culture dish, or test strip.

In an embodiment, a method for treating an angiogenesis-dependent condition comprising administering an effective amount of the isolated and/or recombinant antibody or antigen binding fragment thereof described herein may be provided. In an embodiment, the effective amount may be between about 1 ng and 1,000 ng. In an embodiment, the effective amount may be between about 1 μg and 1,000 μg. In an embodiment, the effective amount may be between about 1 mg and 1,000 mg. In an embodiment, the effective amount may be between about 1 g and 1,000 g. In an embodiment, the condition may be acute respiratory failure, retinopathy, age related macular degeneration, chronic articular rheumatism and psoriasis, disorders associated with inappropriate or inopportune invasion of vessels such as diabetic retinopathy, neovascular glaucoma, restenosis, capillary proliferation in atherosclerotic plaques and osteoporosis, and cancer associated disorders, such as solid tumors, solid tumor metastases, angiofibromas, retrolental fibroplasia, hemangiomas, Kaposi's sarcoma, cancer, optionally, solid tumors. In an embodiment, the condition may be cancer. In an embodiment, the cancer may comprise a solid tumor, optionally a tumor that requires neovascularization to support tumor growth.

In an embodiment, the isolated and/or recombinant antibody or antigen binding fragment thereof may be administered as part of a pharmaceutical composition. In an embodiment, the isolated and/or recombinant antibody is administered as part of a pharmaceutical composition. In an embodiment, the pharmaceutical composition may further comprise a pharmaceutically acceptable excipient, carrier, diluent, vehicle, adjuvant, or a combination thereof. In an embodiment, the adjuvant may be selected from the group consisting of anti-CD40 antibody, imiquimod, resiquimod, GM-CSF, cyclophosphamide, sunitinib, bevacizumab, interferon-alpha, interferon-beta, CpG oligonucleotides and derivatives, poly-(I:C) and derivatives, RNA, sildenafil, particulate formulations with poly(lactide co-glycolide) (PLG), virosomes, or an interleukin (IL).

In an embodiment, the pharmaceutical composition may be administered intravenously, inhalation, subcutaneously, via infusion, orally, intrathecally, intraperitoneally, parenterally, intranasally, or a combination thereof.

In an embodiment, the pharmaceutical composition may be administered at least 1, 2, 3, 4, 5, 6, 7, 8, 9, or 10 times. In an embodiment, the pharmaceutical composition may be administered over the course of 1, 2, 3, 4, 5, 6, or 7 days. In an embodiment, the pharmaceutical composition may be administered over the course of 1, 2, 3, or 4 weeks.

In an embodiment, a kit comprising the isolated and/or recombinant antibody or antigen binding fragment thereof described herein may be provided. In an embodiment, the isolated and/or recombinant antibody or antigen binding fragment thereof may be attached to a solid phase support. In an embodiment, the solid phase support may be an array, a bead, plate, matrix, polymer, test tube, sheet, culture dish, or test strip.

In an embodiment, the use of an effective amount of the isolated and/or recombinant antibody or antigen binding fragment thereof described herein for manufacture of a medicament for the treatment of an angiogenesis-dependent condition. In an embodiment, the condition may be acute respiratory failure, retinopathy, age related macular degeneration, chronic articular rheumatism and psoriasis, disorders associated with inappropriate or inopportune invasion of vessels such as diabetic retinopathy, neovascular glaucoma, restenosis, capillary proliferation in atherosclerotic plaques and osteoporosis, and cancer associated disorders, such as solid tumors, solid tumor metastases, angiofibromas, retrolental fibroplasia, hemangiomas, Kaposi's sarcoma, cancer, optionally, solid tumors. In an embodiment, the condition may be cancer. In an embodiment, the cancer may comprise a solid tumor, optionally a tumor that requires neovascularization to support tumor growth.

In an embodiment, the medicament may comprise a pharmaceutical composition further comprising a pharmaceutically acceptable excipient, carrier, diluent, vehicle, adjuvant, or a combination thereof. In an embodiment, the adjuvant may be selected from the group consisting of anti-CD40 antibody, imiquimod, resiquimod, GM-CSF, cyclophosphamide, sunitinib, bevacizumab, interferon-alpha, interferon-beta, CpG oligonucleotides and derivatives, poly-(I:C) and derivatives, RNA, sildenafil, particulate formulations with poly(lactide co-glycolide) (PLG), virosomes, or an interleukin (IL).

In an embodiment, a composition for treating an angiogenesis-dependent condition comprising an effective amount of the isolated and/or recombinant antibody or antigen binding fragment thereof described herein may be provided. In an embodiment, the condition may be retinopathy, age related macular degeneration, chronic articular rheumatism and psoriasis, disorders associated with inappropriate or inopportune invasion of vessels such as diabetic retinopathy, neovascular glaucoma, restenosis, capillary proliferation in atherosclerotic plaques and osteoporosis, and cancer associated disorders, such as solid tumors, solid tumor metastases, angiofibromas, retrolental fibroplasia, hemangiomas, Kaposi's sarcoma, cancer, optionally, solid tumors, or a combinations thereof. In an embodiment, the condition may be cancer. In an embodiment, the cancer may comprise a solid tumor, optionally a tumor that requires neovascularization to support tumor growth.

In an embodiment, the composition may be a pharmaceutical composition further comprising a pharmaceutically acceptable excipient, carrier, diluent, vehicle, adjuvant, or a combination thereof. In an embodiment, the adjuvant may be selected from the group consisting of anti-CD40 antibody, imiquimod, resiquimod, GM-CSF, cyclophosphamide, sunitinib, bevacizumab, interferon-alpha, interferon-beta, CpG oligonucleotides and derivatives, poly-(I:C) and derivatives, RNA, sildenafil, particulate formulations with poly(lactide co-glycolide) (PLG), virosomes, or an interleukin (IL).

In an embodiment, an isolated and/or recombinant anti-human B cell lymphoma 6 protein antibody or an antigen binding fragment thereof comprising a heavy chain variable (VH) region and a light chain variable (VL) region that specifically binds a human B cell lymphoma 6 protein (BCL-6) polypeptide may be provided. In an embodiment, the antibody or antigen binding fragment thereof may specifically bind to a BCL-6 having at least about 80% sequence homology to an amino acid sequence comprising the amino acid sequence of SEQ ID NO: 1. In an embodiment, the antibody or antigen binding fragment thereof may specifically bind to an epitope on BCL-6 having at least about 80% sequence homology to an amino acid sequence comprising the amino acid sequence of SEQ ID NO: 2. In an embodiment, the sequence homology may be at least about 85%, 90%, 91%, 92%, 93%, 94%, 95%, 96%, 97%, 98%, 99%, or 100% sequence homology.

In an embodiment, the antigen-binding fragment may be an scFv, Fab, Fab′, or F(ab′)2.

In an embodiment, wherein the antibody may be an IgG, IgM, IgE, IgD, or IgA antibody.

In an embodiment, the antibody or antigen binding fragment thereof may be monoclonal. In an embodiment, the antibody or antigen binding fragment thereof may be humanized. In an embodiment, the antibody or antigen binding fragment thereof may be chimeric.

In an embodiment, the antibody or antigen binding fragment thereof may be conjugated to a label, cytotoxic agent, toxin, immunosuppressive agent, or a combination thereof. In an embodiment, the label may be a detectable label. In an embodiment, the detectable label may be a fluorescent label, luminescent label, bioluminescent label, radioactive label, chemiluminescent label, colorimetric label, fluorogenic label, enzymatic label, or a combination thereof.

In an embodiment, an anti-BCL-6 antibody or antigen binding fragment that competes for binding to human B cell lymphoma 6 protein with the antibody or antigen binding fragment described herein may be provided.

In an embodiment, a composition comprising the antibody or antigen binding fragment thereof described herein may be provided. In an embodiment, the composition may be a pharmaceutical composition and further comprises a pharmaceutical excipient, carrier, diluent, adjuvant, or a combination thereof.

In an embodiment, a polynucleotide comprising a nucleic acid sequence comprising at least about 80% sequence homology with a nucleic acid sequence that encodes the amino acid sequence of SEQ ID NO: 1 may be provided. In an embodiment, the sequence homology may be at least about 85%, 90%, 91%, 92%, 93%, 94%, 95%, 96%, 97%, 98%, 99%, or 100% sequence homology. In an embodiment, the polynucleotide may be isolated.

In an embodiment, vector comprising the polynucleotide described herein may be provided. In an embodiment, wherein the vector may be isolated.

In an embodiment, a host cell comprising the vector described herein may be provided. In an embodiment, the host cell is isolated.

In an embodiment, a composition comprising the polynucleotide described herein may be provided. In an embodiment, a composition comprising the vector described herein may be provided. In an embodiment, a composition comprising the host cell described herein may be provided. In an embodiment, the composition may be a pharmaceutical composition and further comprises a pharmaceutically acceptable excipient, carrier, diluent, adjuvant, vehicle, or a combination thereof.

In an embodiment, a method for detecting a human B cell lymphoma 6 protein polypeptide comprising contacting a sample with the antibody or antigen binding fragment thereof described herein may be provided. In an embodiment, the antibody or antigen binding fragment thereof may be attached to a solid phase support. In an embodiment, the solid phase support may be an array, a bead, plate, matrix, polymer, test tube, sheet, culture dish, or test strip.

In an embodiment, a method for treating an angiogenesis-dependent condition comprising administering an effective amount of the antibody or antigen binding fragment thereof described herein may be provided. In an embodiment, the condition may be acute respiratory failure, retinopathy, age related macular degeneration, chronic articular rheumatism and psoriasis, disorders associated with inappropriate or inopportune invasion of vessels such as diabetic retinopathy, neovascular glaucoma, restenosis, capillary proliferation in atherosclerotic plaques and osteoporosis, and cancer associated disorders, such as solid tumors, solid tumor metastases, angiofibromas, retrolental fibroplasia, hemangiomas, Kaposi's sarcoma, cancer, optionally, solid tumors.

In an embodiment, the antibody, or antigen binding fragment thereof may be administered as part of a pharmaceutical composition.

In an embodiment, the effective amount may be between about 1 ng and 1,000 ng. In an embodiment, the effective amount may be between about 1 μg and 1,000 μg. In an embodiment, the effective amount may be between about 1 mg and 1,000 mg. In an embodiment, the effective amount may be between about 1 g and 1,000 g.

In an embodiment, the antibody or antigen binding fragment thereof may be administered intravenously, inhalation, subcutaneously, via infusion, orally, intrathecally, intraperitoneally, parenterally, intranasally, or a combination thereof. In an embodiment, may be administered at least 1, 2, 3, 4, 5, 6, 7, 8, 9, or 10 times. In an embodiment, the antibody or antigen binding fragment thereof may be administered over the course of 1, 2, 3, 4, 5, 6, or 7 days. In an embodiment, the antibody or antigen binding fragment thereof may be administered over the course of 1, 2, 3, or 4 weeks.

In an embodiment, a kit comprising the antibody or antigen binding fragment thereof described herein may be provided. In an embodiment, the antibody or antigen binding fragment thereof may be attached to a solid phase support. In an embodiment, the solid phase support may be an array, a bead, plate, matrix, polymer, test tube, sheet, culture dish, or test strip.

In an embodiment, the use of an effective amount of the antibody or antigen binding fragment thereof described herein for manufacture of a medicament for the treatment of an angiogenesis-dependent condition may be provided. In an embodiment, the condition may be acute respiratory failure, retinopathy, age related macular degeneration, chronic articular rheumatism and psoriasis, disorders associated with inappropriate or inopportune invasion of vessels such as diabetic retinopathy, neovascular glaucoma, restenosis, capillary proliferation in atherosclerotic plaques and osteoporosis, and cancer associated disorders, such as solid tumors, solid tumor metastases, angiofibromas, retrolental fibroplasia, hemangiomas, Kaposi's sarcoma, cancer, optionally, solid tumors.

In an embodiment, the antibody or antigen binding fragment thereof may be formulated for intravenous, subcutaneous, inhalation, preferably via the intranasal route, infusion, oral, intrathecal, intraperitoneal, parenteral administration, or a combination thereof. In an embodiment, the antibody may be an IgG, IgM, IgE, IgD, or IgA antibody.

In an embodiment, a composition for treating an angiogenesis-dependent condition comprising an effective amount of the antibody or antigen binding fragment thereof described herein may be provided. In an embodiment, the condition may be retinopathy, age related macular degeneration, chronic articular rheumatism and psoriasis, disorders associated with inappropriate or inopportune invasion of vessels such as diabetic retinopathy, neovascular glaucoma, restenosis, capillary proliferation in atherosclerotic plaques and osteoporosis, and cancer associated disorders, such as solid tumors, solid tumor metastases, angiofibromas, retrolental fibroplasia, hemangiomas, Kaposi's sarcoma, cancer, optionally, solid tumors, or a combinations thereof. In an embodiment, the condition may be cancer. In an embodiment, the cancer may comprise a solid tumor, optionally a tumor that requires neovascularization to support tumor growth.

In an embodiment, the composition may be a pharmaceutical composition further comprising a pharmaceutically acceptable excipient, carrier, diluent, vehicle, adjuvant, or a combination thereof.

In an embodiment, the effective amount may be between about 1 ng and 1,000 ng. In an embodiment, the effective amount may be between about 1 ng and 1,000 ng. In an embodiment, the effective amount may be between about 1 mg and 1,000 mg. In an embodiment, the effective amount may be between about 1 g and 1,000 g.

In an embodiment, the antibody or antigen binding fragment thereof may be formulated for intravenous, inhalation, preferably via the intranasal route, subcutaneous, infusion, oral, intrathecal, intraperitoneal, parenteral administration, or a combination thereof.

In an embodiment, an isolated peptide comprising an amino acid sequence that shares at least about 80% sequence homology with the amino acid sequence of SEQ ID NO: 1 may be provided. In an embodiment, an isolated peptide comprising an amino acid sequence that shares at least about 80% sequence homology with the amino acid sequence of SEQ ID NO: 2 may be provided. In an embodiment, the sequence homology may be at least about 81%, 82%, 83%, 84%, 85%, 86%, 87%, 88%, 89%, 90%, 91%, 92%, 93%, 94%, 95%, 96%, 97%, 98%, 99%, or 100%.

In an embodiment, a composition comprising the isolated peptide described herein may be provided. In an embodiment, the composition may be a pharmaceutical composition. In an embodiment, the composition may further comprise an excipient, carrier, diluent, adjuvant, or a combination thereof.

In an embodiment, a pharmaceutical salt of the isolated peptide described herein may be provided. In an embodiment, the pharmaceutically acceptable salt may be a chloride salt, acetate salt, or trifluoro-acetate salt.

In an embodiment, a fusion protein comprising the isolated peptide described herein may be provided.

In an embodiment, a MHC-peptide complex comprising the peptide described herein complexed with an MHC may be provided.

In an embodiment, a method of making a hybridoma cell that produces a monoclonal antibody that specifically binds BCL-6 comprising immunizing a mouse with a BCL-6 antigen, optionally a peptide comprising an amino acid sequence of with at least about 80% sequence homology with the amino acid sequence of SEQ ID NO: 1, 2, or a combination thereof, fusing a B cell from the immunized mouse with a myeloma cell to produce a monoclonal antibody producing hybridoma cell may be provided.

In an embodiment, a method for producing an antibody that specifically binds to antigen consisting of an amino acid sequence of with at least about 80% sequence homology with the amino acid sequence of SEQ ID NO: 1, 2, or a combination thereof may be provided, the method comprising immunizing a genetically engineered non-human mammal with the antigen, isolating mRNA molecules from antibody producing cells of the non-human mammal, producing a phage display library displaying protein molecules encoded by the mRNA molecules, and isolating at least one phage from the phage display library, wherein the at least one phage displays the antibody specifically binding to the antigen. In an embodiment, the antibody may be a monoclonal antibody or a chimeric antibody. In an embodiment, the antibody may be a monoclonal antibody. In an embodiment, the antibody may be a chimeric antibody. In an embodiment, the method may further comprise humanizing the antibody. In an embodiment, the non-human mammal is mouse.

In an embodiment, a peptide comprising at least 90% sequence identity to an amino acid sequence comprising TKVQYRVSATDLPPELPKAC (SEQ ID NO: 2) may be provided. In an embodiment, the peptide may comprise 100% sequence identity to TKVQYRVSATDLPPELPKAC (SEQ ID NO: 2). In an embodiment, the peptide may be in the form of a pharmaceutically acceptable salt.

In an embodiment, a composition comprising the peptides described herein may be provided, wherein the composition may comprise an adjuvant, a pharmaceutically acceptable carrier, or both. In an embodiment, the peptide may be in the form of a chloride or acetate salt.

In an embodiment, the adjuvant may be selected from the group consisting of anti-CD40 antibody, imiquimod, resiquimod, GM-CSF, cyclophosphamide, sunitinib, bevacizumab, interferon-alpha, interferon-beta, CpG oligonucleotides and derivatives, poly-(I:C) and derivatives, RNA, sildenafil, particulate formulations with poly(lactide co-glycolide) (PLG), virosomes, or an interleukin (TL).

In an embodiment, a nucleic acid may encode the peptide, protein, or antibody or antigen binding fragment thereof described herein.

In an embodiment, the nucleic acid may be isolated, engineered, and/or recombinant.

In an embodiment, a vector may comprise the nucleotide described herein.

In an embodiment, the vector may be isolated, engineered, and/or recombinant.

In an embodiment, a host cell may comprise the vector described herein.

In an embodiment, the cell may be isolated, engineered, and/or recombinant.

In an embodiment, a composition may comprise the peptide, protein, or antibody or antigen binding fragment thereof described herein.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements.

FIG. 1 depicts the Cancer Genome Atlas (TCGA) database for Bcl-6 expression in human tumors. Values in this dataset are generated by first combining gene expression RNAseq values of all TCGA cohorts and then mean normalizing all values per gene. This data was then divided into the individual cancer type after normalization so that this data is available for each cancer type. Since there are 30-40 cancer types with RNAseq data, the TCGA Pan-Cancer Atlas data can serve as a proxy of background distribution of gene expression. The results indicate that Bcl-6 is highly expressed in many cancer types.

FIG. 2 sets forth the annotated sequence of a variable light (FIG. 2A) and variable heavy (FIG. 2B) chains of an exemplary recombinant and/or isolated antibody or an antigen binding fragment thereof that specifically binds a human B cell lymphoma 6 protein (BCL-6).

FIG. 3 depicts lysates from human cell lines and cell media supernatants subjected to RNA extraction and qPCR (FIG. 3A) or ELISA assay (FIG. 3B), respectively, for the presence of Bcl-6. Lysates of a panel of human cell lines (human endothelial cells, bladder cells, prostate cell, lung cells, colon cells and breast cells) were subjected to quantitative PCR using primers towards Bcl-6. Bcl-6 levels were highest in prostate (LNCaP and PC-3) and bladder (UROtsa and UMUC-3). Supernatants from these same cell lines were analyzed by ELISA assay towards Bcl-6. Secreted Bcl-6 was noted in all cell types (endothelial cells, bladder cells, prostate cell, lung cells, colon cells and breast cells) with the highest seen associated with endothelial cells.

FIG. 4 depicts the ability of the neutralizing anti-Bcl-6 antibody ability described herein to inhibit the ability of endothelial cells, human dermal microvascular endothelial cells (HDMEC) [FIG. 4A, FIG. 4B, FIG. 4C] and human umbilical vein endothelial cells (HUVEC) [FIG. 4D, FIG. 4E, FIG. 4F] to form capillaries. Neutralizing Bcl-6 antibody described herein inhibited the ability of endothelial cells HDMEC and HUVEC to form capillaries. IgG serves as a negative control and AVASTIN® (bevacizumab) as a positive control. (A) HDMEC, no treatment; (B) HDMEC, treated with 0.7 μg/mL normal rabbit IgG; and (C) HDMEC treated with 0.7 mg/mL anti-BCL-6. (D) HUVEC, no treatment; (E) HUVEC, treated with 0.7 μg/mL normal rabbit IgG; and (C) HUVEC treated with 0.7 mg/mL anti-BCL-6. Each is a representative photograph from 96 well tube formation assay.

FIG. 5 depicts a graphical representation of tube formation assay (FIG. 5A) and tube disruption (FIG. 5B) for human dermal microvascular endothelial cells (HDMEC) and human umbilical vein endothelial cells (HUVEC) for no treatment, human IgG, AVASTIN® (bevacizumab, FDA approved drug, comparative control), rabbit IgG, or anti-BCL-6 antibody described herein. Both bevacizumab and anti-Bcl-6 antibody inhibited tube formation (which is generation of new blood vessels), while they have no effect on tube disruption (breakdown of existing blood vessels).

FIG. 6A and FIG. 6B show that neutralizing antibodies to Bcl-6 described herein reduced cellular viability of HUVEC and HDMEC cells. Recombinant Bcl-6 was added to human endothelial cells and cell viability monitored. The addition of recombinant Bcl-6 did not increase the viability or proliferative ability of these cells. Alternatively, neutralizing antibody to Bcl-6 reduced cellular viability of both HDMEC and HUVEC cells like what is seen with bevacizumab (FDA approved drug, comparative control), a neutralizing monoclonal antibody towards vascular endothelial growth factor (VEGF). Specifically, about 50-times lower amount of neutralizing antibody to Bcl-6 demonstrated the same efficacy as bevacizumab.

FIG. 7 depicts data showing that the neutralizing antibodies to Bcl-6 described herein reduced cellular number of human bladder cancer cell line T24 after 72 hours of incubation, suggesting that the neutralizing antibodies to Bcl-6 inhibited cancer cellular proliferation.

FIG. 8 depicts data showing that patients with bladder cancer had more Bcl-6 in the urine than controls.

FIG. 9A is a graph depicting cellular proliferation of T24 parental cells measured by MTT assay at 72 hrs after treatment with increasing concentrations of anti-Bcl-6 antibody disclosed herein. FIG. 9B is a graph depicting capillary tube formation in HUVECs cultured on growth factor-reduced Matrigel in the presence of anti-Bcl-6 antibody disclosed herein, bevacizumab, control IgG, or no treatment.

FIG. 10A and FIG. 10B are a photograph (FIG. 10A) and a graph (FIG. 10B) showing that tumors treated with anti-Bcl-6 antibody disclosed herein were significantly smaller than control untreated tumors.

### DETAILED DESCRIPTION

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments, and is not intended to be limiting. Instead, the scope of the present disclosure will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

### Definitions

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Antibodies are heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains (Clothia et al. J. Mol. Biol. 186:651 (1985); Novotny and Haber, Proc. Natl. Acad. Sci. U.S.A. 82:4592 (1985), incorporated herein by reference in its entirety).

The term “variable” refers broadly to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a 3-sheet configuration, connected by three CDRs, which form loops connecting, and in some cases forming part of, the 3-sheet structure. The CDRs in each chain are held together in close proximity by the FR regions and, with the CDRs from the other chain, contribute to the formation of the antigen-binding site of antibodies. See Kabat et al. Sequences of Proteins of Immunological Interest, Fifth Edition, National Institute of Health, Bethesda, MD. (1991), incorporated herein by reference in its entirety. The constant domains are not involved directly in binding an antibody to an antigen, but exhibit various effector functions, such as participation of the antibody in antibody-dependent cellular toxicity.

As used herein, the term “constant region” or “constant domain” are interchangeable and refer to an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain. In embodiments, an antibody or antigen-binding fragment comprises a constant region or portion thereof that is sufficient for antibody-dependent cell-mediated cytotoxicity (ADCC).

As used herein, the term “variable region” or “variable domain” are interchangeable. The variable region may comprise three complementarity-determining regions (CDRs) and may comprise four framework regions (FRs).

As used herein, the term “heavy chain” when used in reference to an antibody can refer to any distinct type, e.g., alpha (α), delta (δ), epsilon (ε), gamma (γ), or mu (μ), based on the amino acid sequence of the constant domain, which give rise to IgA, IgD, IgE (e.g., IgG, e.g., IgG1, IgG2, IgG3, and IgG4), IgG, and IgM classes of antibodies, respectively.

As used herein, the term “light chain” when used in reference to an antibody can refer to any distinct type, e.g., kappa (κ) or lambda (λ), based on the amino acid sequence of the constant domains.

In embodiments, anti-BCL-6 antibodies or fragments thereof (including molecules which comprise, or alternatively consist of, antibody fragments or variants thereof) comprise all or a portion of a constant region of a heavy chain of an antibody. Heavy chain constant region amino acid sequences are well known in the art. In embodiments, the heavy chain is a mammalian, human, primate, bovine, porcine, murine (e.g., mouse or rat), or lapine (e.g., rabbit) heavy chain. In embodiments, the heavy chain is a rabbit heavy chain. In embodiments, the heavy chain is a human heavy chain. In embodiments, the heavy chain is an alpha (α), delta (δ), epsilon (ε), gamma (γ), or mu (μ) heavy chain. In embodiments, the heavy chain is a gamma (γ) heavy chain.

In embodiments, anti-BCL-6 antibodies or fragments thereof (including molecules which comprise, or alternatively consist of, antibody fragments or variants thereof) comprise all or a portion of a constant region of a light chain of an antibody. Light chain constant region amino acid sequences are well known in the art. In embodiments, the light chain is a mammalian, human, primate, bovine, porcine, murine (e.g., mouse or rat), or lapine (e.g., rabbit) light chain. In embodiments, the light chain is a rabbit light chain. In embodiments, the light chain is a human light chain. In embodiments, the light chain is a kappa (κ) or lambda (λ) light chain. In embodiments, the light chain is a kappa (κ) light chain.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′) 2 fragment that has two antigen-combining sites and is still capable of cross-linking antigen.

“Fv” is the minimum antibody fragment which contains a complete antigen-recognition and binding site. In a two-chain Fv species, this region consists of a dimer of one heavy and one light chain variable domain in tight, non-covalent association. In a single-chain Fv (scFv) species, one heavy and one light chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′) 2 antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG 1, IgG2, IgG3, IgG4, IgA1, and IgA2. The heavy-chain constant domains that correspond to the different classes of immunoglobulins are called α, δ, ε, γ, and μ, respectively. In addition, secretory immunoglobulin A (IgA), the predominant immunoglobulin isotype present in airway secretions, is composed of two IgA molecules (dimeric IgA), a joining protein (J chain), and a secretory component. The subunit structures and three-dimensional configurations of different classes of immunoglobulins are well known. “Therapeutic Antibody Engineering” (1^{st} Ed.) Strohl & Strohl Woodhead Publishing (2012), incorporated herein by reference in its entirety. The term “antibody” specifically encompasses monoclonal antibodies, including antibody fragment clones. The anti-BCL-6 antibodies described herein comprise CDRs specifically bind to an epitope in the amino acid sequences of SEQ ID NO: 1, preferably the amino acid sequence of SEQ ID NO: 2.

“Antibody fragments” comprise a portion of an intact antibody, generally the antigen binding or variable region of the intact antibody. Examples of antibody fragments include Fab, Fab′, F(ab′) 2, and Fv fragments; diabodies; single-chain antibody molecules, including single-chain Fv (scFv) molecules; and multispecific antibodies formed from antibody fragments. “Human Monoconal Antibodies: Methods and Protocols” (2^{nd} Ed.) Steinitz (Ed.) Humana Press (2019) incorporated herein by reference in its entirety.

The term “monoclonal antibody” as used herein refers to an antibody (or antibody fragment) obtained from a population of substantially homogeneous antibodies, e.g., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they may be synthesized, for example, by hybridoma culture or via recombinant DNA methods, and therefore are usually uncontaminated by other immunoglobulins. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by the hybridoma method first described by Kohler et al. Nature 256:495 (1975), incorporated herein by reference in its entirety, or may be made by recombinant DNA methods (see, e.g., U.S. Pat. No. 4,816,567, incorporated herein by reference in its entirety). The “monoclonal antibodies” also include clones of antigen-recognition and binding-site containing antibody fragments (Fv clones) isolated from phage antibody libraries using the techniques described in, for example, Clackson, et al. Nature 352:624-628 (1991) and Marks et al. J. Mol. Biol. 222:581-597 (1991), each of which is incorporated herein by reference in its entirety.

Monoclonal antibodies of the invention may be prepared using hybridoma methods. In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods, such as those described in U.S. Pat. No. 4,816,567, incorporated herein by reference in its entirety. DNA encoding the monoclonal antibodies of the invention can be readily isolated and sequenced using conventional procedures (e.g., by using oligonucleotide probes that are capable of binding specifically to genes encoding the heavy and light chains of murine antibodies).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. U.S. Pat. No. 4,816,567; Morrison et al. Proc. Natl. Acad. Sci. USA, 81:6851-6855 (1984); “Antibody Engineering” Volume 2 (2^{nd} Ed.) Kontermann & Dubel. Springer Press (2010), each of which is incorporated herein by reference in its entirety.

A “human” antibody (also called a “fully human” antibody) is an antibody that includes human framework regions and all of the CDRs from a human immunoglobulin. In one example, the framework and the CDRs are from the same originating human heavy and/or light chain amino acid sequence. However, frameworks from one human antibody can be engineered to include CDRs from a different human antibody.

“Humanized” forms of non-human (e.g., murine) antibodies are chimeric immunoglobulins, immunoglobulin chains or fragments thereof (such as Fv, Fab, Fab′, F(ab′) 2 or other antigen-binding subsequences of antibodies) which contain minimal sequence derived from non-human immunoglobulin. For the most part, humanized antibodies are human immunoglobulins (recipient antibody) in which residues from a complementarity-determining region (CDR) of the recipient are replaced by residues from a CDR of a non-human species (such as mouse, rat or rabbit) or a synthetic sequence (donor antibody), having the desired specificity, affinity, and capacity. In some instances, Fv framework region (FR) residues of the human immunoglobulin are replaced by corresponding non-human residues. Furthermore, humanized antibodies may comprise residues which are found neither in the recipient antibody nor in the imported CDR or framework sequences. These modifications may be made to further refine and optimize antibody performance. In some embodiments, all the CDRs are from the donor immunoglobulin in a humanized immunoglobulin. Constant regions need not be present, but if they are, they should be substantially identical to human immunoglobulin constant regions, e.g., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Humanized immunoglobulins can be constructed by art-known means, such as genetic engineering, framework assembly, and combinatorial engineering. See for example, U.S. Pat. Nos. 5,585,089; 5,565,332; 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,225,539; 4,816,567; 6,054,297; and 9,090,994, U.S. Patent Application No. 2005/0042664, each of which is incorporated herein by reference in its entirety.

Numerous methods for humanization of an antibody sequence are known in the art; see e.g. the review by Almagro & Fransson (2008) Front Biosci. 13: 1619-1633, incorporated herein by reference in its entirety. One commonly used method is CDR grafting, or antibody reshaping, which involves grafting of the CDR sequences of a donor antibody, such as a murine (e.g., mouse or rat) or lapine (e.g., rabbit) antibody, into the framework scaffold of a human antibody of different specificity. Since CDR grafting may reduce the binding specificity and affinity, and thus the biological activity, of a CDR grafted non-human antibody, back mutations may be introduced at selected positions of the CDR grafted antibody in order to retain the binding specificity and affinity of the parent antibody. Identification of positions for possible back mutations can be performed using information available in the literature and in antibody databases. An alternative humanization technique to CDR grafting and back mutation is resurfacing, in which non-surface exposed residues of non-human origin are retained, while surface residues are altered to human residues. Another alternative technique is known as “guided selection” (Jespers et al. (1994) Biotechnology 12, 899, incorporated herein by reference in its entirety) and can be used to derive from for example a murine or rat antibody a fully human antibody conserving the epitope and binding characteristics of the parental antibody. A further method of humanization is the so-called 4D humanization. The 4D humanization protocol is described in the patent application US20110027266 A1 (WO2009032661A1), each of which incorporated herein by reference in its entirety, and is exemplified in the following applying the 4D humanization to humanize the rat antibody variable light (V_{L}) and heavy (V_{H}) domains.

For chimeric antibodies, humanization typically involves modification of the framework regions of the variable region sequences.

Amino acid residues that are part of a CDR will typically not be altered in connection with humanization, although in certain cases it may be desirable to alter individual CDR amino acid residues, for example to remove a glycosylation site, a deamidation site, an isomerization site, or an undesired cysteine residue. N-linked glycosylation occurs by attachment of an oligosaccharide chain to an asparagine residue in the tripeptide sequence Asn-X-Ser or Asn-X-Thr, where X may be any amino acid except Pro. Removal of an N-glycosylation site may be achieved by mutating either the Asn or the Ser/Thr residue to a different residue, in particular by way of conservative substitution. Deamidation of asparagine and glutamine residues can occur depending on factors such as pH and surface exposure. Asparagine residues are particularly susceptible to deamidation, primarily when present in the sequence Asn-Gly, and to a lesser extent in other dipeptide sequences such as Asn-Ala. When such a deamidation site, in particular Asn-Gly, is present in a CDR sequence, it may therefore be desirable to remove the site, typically by conservative substitution to remove one of the implicated residues. Substitution in a CDR sequence to remove one of the implicated residues is also intended to be encompassed by the present invention.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv see Pluckthun, in The Pharmacology of Monoclonal Antibodies, vol. 113, Rosenburg and Moore (Eds.), Springer-Verlag, New York, pp. 269-315 (1994), incorporated herein by reference in its entirety.

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP Patent No. 2,158,221; WO 93/11161; and Hollinger et al. Proc. Natl. Acad. Sci. USA 90:6444-6448 (1993), incorporated herein by reference in its entirety.

An “isolated” antibody, peptide, protein, cell, nucleic acid, or vector is one that has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

A “recombinant” nucleic acid or vector is one that has been formed by laboratory methods of genetic recombination, e.g., molecular cloning, or one that comprises such recombinant material. Genetic recombination generally creates sequences that would not otherwise be found in the genome. A “recombinant” antibody, peptide, or protein is one that is encoded by a recombinant nucleic acid(s). A “recombinant” cell is one that contains, comprises, or expresses a recombinant nucleic acid(s), recombinant vector, and/or recombinant antibody, peptide and/or protein.

An “engineered” nucleic acid, vector, antibody, peptide, or protein is one that is created and/or manipulated via human or artificial intelligence engineering. An “engineered” cell is one that is created and/or manipulated via human or artificial intelligence engineering and/or contains, comprises, and/or expresses an engineered nucleic acid(s), recombinant vector, and/or recombinant antibody, peptide and/or protein.

The term “variant” as used herein refers broadly to a polypeptide that possesses a similar or identical function as a BCL-6 polypeptide, an anti-BCL-6 antibody or antibody fragment thereof, but does not necessarily comprise a similar or identical amino acid sequence of a BCL-6 polypeptide, anti-BCL-6 or antibody fragment thereof, or possess a similar or identical structure of a BCL-6 polypeptide, an anti-BCL-6 antibody or antibody fragment thereof. A variant having a similar amino acid identity refers to a polypeptide that satisfies at least one of the following: (a) a polypeptide comprising, or alternatively consisting of, an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of a BCL-6 polypeptide, anti-BCL-6 or antibody fragment thereof (including a VH domain, VHCDR, VL domain, or VLCDR having an amino acid sequence of any one of those described herein); (b) a polypeptide encoded by a nucleotide sequence, the complementary sequence of which hybridizes under stringent conditions to a nucleotide sequence encoding a BCL-6 polypeptide or fragment thereof, an anti-BCL-6 antibody or antibody fragment thereof (including a VH domain, VHCDR, VL domain, or VLCDR having an amino acid sequence of any one of those described herein), of at least 5 amino acid residues, at least 10 amino acid residues, at least 15 amino acid residues, at least 20 amino acid residues, at least 25 amino acid residues, at least 30 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues; and (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, identical to the nucleotide sequence encoding a BCL-6 polypeptide or fragment thereof, an anti-BCL-6 antibody or antibody fragment thereof (including a VH domain, VHCDR, VL domain, or VLCDR having an amino acid sequence described herein). A polypeptide with similar structure to a BCL-6 polypeptide or fragment thereof, an anti-BCL-6 antibody or antibody fragment thereof, described herein refers to a polypeptide that has a similar secondary, tertiary or quaternary structure of a BCL-6 polypeptide or fragment thereof, an anti-BCL-6 antibody, or antibody fragment thereof, described herein. The structure of a polypeptide can be determined by methods known to those skilled in the art, including but not limited to, X-ray crystallography, nuclear magnetic resonance, and crystallographic electron microscopy. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide at the corresponding position in the second sequence, then the molecules are identical at that position. The percent identity between the two sequences is a function of the number of identical positions shared by the sequences (e.g., % identity=number of identical overlapping positions/total number of positions×100%). In one embodiment, the two sequences are the same length.

The determination of percent identity between two sequences can be accomplished using a mathematical algorithm known to those of skill in the art. An example of a mathematical algorithm for comparing two sequences is the algorithm of Karlin and Altschul Proc. Natl. Acad. Sci. USA 87:2264-2268 (1990), incorporated herein by reference in its entirety, modified as in Karlin and Altschul Proc. Natl. Acad. Sci. USA 90:5873-5877 (1993), incorporated herein by reference in its entirety. The BLASTn and BLASTx programs of Altschul, et al. J. Mal. Biol. 215:403-410 (1990), incorporated herein by reference in its entirety, have incorporated such an algorithm. BLAST nucleotide searches can be performed with the BLASTn program, score=100, wordlength=12 to obtain nucleotide sequences homologous to a nucleic acid molecule described herein. BLAST protein searches can be performed with the BLASTx program, score=50, wordlength=3 to obtain amino acid sequences homologous to a protein molecule described herein. To obtain gapped alignments for comparison purposes, Gapped BLAST can be utilized as described in Altschul et al. Nucleic Acids Res. 25: 3389-3402 (1997). Alternatively, PSI-BLAST can be used to perform an iterated search which detects distant relationships between molecules (Id.). When utilizing BLAST, Gapped BLAST, and PSI-BLAST programs, the default parameters of the respective programs (e.g., BLASTx and BLASTn) can be used. (See ncbi.nlm.nih.gov).

Another example of a mathematical algorithm utilized for the comparison of sequences is the algorithm of Myers and Miller, CABIOS (1989). The ALIGN program (version 2.0) which is part of the GCG sequence alignment software package has incorporated such an algorithm. Other algorithms for sequence analysis known in the art include ADVANCE and ADAM as described in Torellis and Robotti Comput. Appl. Biosci., 10:3-5(1994), incorporated herein by reference in its entirety; and FASTA described in Pearson and Lipman Proc. Natl. Acad. Sci. 85:2444-8(1988), incorporated herein by reference in its entirety. Within FASTA, ktup is a control option that sets the sensitivity and speed of the search.

Amino acid substitutions may be conservative or non-conservative. Preferably, substitutions are conservative substitutions, in which one amino acid is substituted for another amino acid with similar structural and/or chemical properties.

“Conservative” amino acid substitutions are those substitutions that do not substantially affect or decrease the affinity of a protein, such as an antibody to BCL-6. For example, a monoclonal antibody that immunospecifically binds BCL-6 can include at most about 1, at most about 2, at most about 5, at most about 10, or at most about 15 conservative substitutions and immunospecifically bind a BCL-6 polypeptide. The term “conservative variant” also includes the use of a substituted amino acid in place of an unsubstituted parent amino acid, provided that antibody immunospecifically binds BCL-6. Non-conservative substitutions are those that reduce an activity or binding to BCL-6.

Conservative amino acid substitution tables providing functionally similar amino acids are well known to one of ordinary skill in the art. Conservative substitutions may comprise those, which are described by Dayhoff in “The Atlas of Protein Sequence and Structure. Vol. 5”, Natl. Biomedical Research, incorporated herein by reference in its entirety. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

For example, in an embodiment, amino acids, which belong to one of the following groups, can be exchanged for one another, thus, constituting a conservative exchange: Group 1: alanine (A), proline (P), glycine (G), asparagine (N), serine (S), threonine (T); Group 2: cysteine (C), serine (S), tyrosine (Y), threonine (T); Group 3: valine (V), isoleucine (I), leucine (L), methionine (M), alanine (A), phenylalanine (F); Group 4: lysine (K), arginine (R), histidine (H); Group 5: phenylalanine (F), tyrosine (Y), tryptophan (W), histidine (H); and Group 6: aspartic acid (D), glutamic acid (E). In an embodiment, a conservative amino acid substitution may be selected from the following of T→A, G→A, A→I, T→V, A→M, T→I, A→V, T→G, and/or T→S.

A conservative amino acid substitution may comprise the substitution of an amino acid by another amino acid of the same class, for example, (1) nonpolar: Ala, Val, Leu, Ile, Pro, Met, Phe, Trp; (2) uncharged polar: Gly, Ser, Thr, Cys, Tyr, Asn, Gln; (3) acidic: Asp, Glu; and (4) basic: Lys, Arg, His. Other conservative amino acid substitutions may also be made as follows: (1) aromatic: Phe, Tyr, His; (2) proton donor: Asn, Gln, Lys, Arg, His, Trp; and (3) proton acceptor: Glu, Asp, Thr, Ser, Tyr, Asn, Gln (see, for example, U.S. Pat. No. 10,106,805, the contents of which are incorporated by reference in their entirety).

Conservative substitutions may be made in accordance with Table A. Methods for predicting tolerance to protein modification may be found in, for example, Guo et al., Proc. Natl. Acad. Sci., USA, 101(25):9205-9210 (2004), the contents of which are incorporated by reference in their entirety.

TABLE A
Conservative Amino Acid substitution
Conservative Amino Acid Substitutions

| Amino Acid | Substitutions (others are known in the art) |
|---|---|
| Ala | Ser, Gly, Cys |
| Arg | Lys, Gln, His |
| Asn | Gln, His, Glu, Asp |
| Asp | Glu, Asn, Gln |
| Cys | Ser, Met, Thr |
| Gln | Asn, Lys, Glu, Asp, Arg |
| Glu | Asp, Asn, Gln |
| Gly | Pro, Ala, Ser |
| His | Asn, Gln, Lys |
| Ile | Leu, Val, Met, Ala |
| Leu | Ile, Val, Met, Ala |
| Lys | Arg, Gln, His |
| Met | Leu, Ile, Val, Ala, Phe |
| Phe | Met, Leu, Tyr, Trp, His |
| Ser | Thr, Cys, Ala |
| Thr | Ser, Val, Ala |
| Trp | Tyr, Phe |
| Tyr | Trp, Phe, His |
| Val | Ile, Leu, Met, Ala, Thr |

The sequences described herein may comprise 1, 2, 3, 4, 5, 10, 15, 20, 25, or 30 amino acid or nucleotide mutations, substitutions, deletions. Any one of SEQ ID NO: 1-22 may comprise 1, 2, 3, 4, 5, 10, 15, 20, 25, or 30 mutations, substitutions, or deletions. In another aspect, any one of SEQ ID NO: 1-22 may comprise at most 1, 2, 3, 4, 5, 10, 15, 20, 25, or 30 mutations, substitutions, or deletions. In another aspect, any one of SEQ ID NO: 1-22 may comprise at most 1, 2, 3, 4, or 5 mutations, substitutions, or deletions. In another aspect, any one of SEQ ID NO: 1-22 may comprise at most 1, 2, or 3 mutations, substitutions, or deletions. In another aspect, any one of SEQ ID NO: 1-22 may comprise at most 1 or 2 mutations, substitutions, or deletions. In another aspect, any one of SEQ ID NO: 1-22 may comprise at most 1 mutation, substitution, or deletion. In an aspect, the mutations or substitutions may be conservative amino acid substitutions. In another aspect, any one of SEQ ID NO: 1-22 may comprise at most 1, 2, 3, 4, or 5 conservative substitutions. In another aspect, any one of SEQ ID NO: 1-22 may comprise at most 1, 2, or 3 conservative substitutions. In another aspect, any one of SEQ ID NO: 1-22 may comprise at most 1 or 2 conservative substitutions. In another aspect, any one of SEQ ID NO: 1-22 may comprise at most 1 conservative substitution.

Conservative substitutions in the polypeptides described herein may be those shown in Table B under the heading of “conservative substitutions.” If such substitutions result in a change in biological activity, then more substantial changes, denominated “exemplary substitutions” in Table B, may be introduced and the products screened if needed

TABLE B
Amino Acid substitution
Amino Acid Substitutions

| Original Residue | | |
|---|---|---|
| (naturally occurring | Conservative | |
| amino acid) | Substitutions | Exemplary Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; |
| | | Norleucine |
| Leu (L) | Ile | Norleucine; Ile; Val; Met; |
| | | Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; |
| | | Norleucine |

“Derivative” as used herein, refers to a variant polypeptide described herein that comprises, or alternatively consists of, an amino acid sequence of a BCL-6 polypeptide or fragment thereof, or an antibody described herein that immunospecifically binds to BCL-6, which has been altered by the introduction of amino acid residue substitutions, deletions or additions. The term “derivative” as used herein also refers to a BCL-6 polypeptide or fragment thereof, an antibody that immunospecifically binds to BCL-6 which has been modified, e.g., by the covalent attachment of any type of molecule to the polypeptide. For example, but not by way of limitation a BCL-6 polypeptide or fragment thereof, or an anti-BCL-6 antibody, may be modified, e.g., by glycosylation, acetylation, pegylation, phosphorylation, amidation, derivatization by known protecting/blocking groups, proteolytic cleavage, linkage to a cellular ligand or other protein, etc. A derivative of a BCL-6 polypeptide or fragment thereof, or an anti-BCL-6 antibody or fragment thereof, may be modified by chemical modifications using techniques known to those of skill in the art, including, but not limited to, specific chemical cleavage, acetylation, formylation, metabolic synthesis of tunicamycin, etc. Further, a derivative of a BCL-6 polypeptide or fragment thereof, or an anti-BCL-6 antibody or fragment thereof, may contain one or more non-classical amino acids. A polypeptide derivative possesses a similar or identical function as a BCL-6 polypeptide or fragment thereof, or an anti-BCL-6 antibody or fragment thereof, described herein.

“Epitopes” as used herein refers to portions of BCL-6 having antigenic or immunogenic activity in an animal, preferably a mammal. An epitope having immunogenic activity is a portion of BCL-6 that elicits an antibody response in an animal. An epitope having antigenic activity is a portion of BCL-6 to which an antibody immunospecifically binds as determined by any method known in the art, for example, by the immunoassays described herein. Antigenic epitopes need not necessarily be immunogenic.

“Fragment” as used herein refers broadly to a polypeptide comprising an amino acid sequence of at least 5 amino acid residues, at least 10 amino acid residues, at least 15 amino acid residues, at least 20 amino acid residues, at least 25 amino acid residues, at least 30 amino acid residues, at least 35 amino acid residues, at least 40 amino acid residues, at least 45 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues, of the amino acid sequence of a BCL-6, or an anti-BCL-6 antibody (including molecules such as scFvs, that comprise, or alternatively consist of, antibody fragments or variants thereof) that immunospecifically binds to BCL-6 comprising the amino acid sequence of SEQ ID NO: 1, preferably the amino acid sequence of SEQ ID NO: 2.

“Fusion protein,” as used herein, refers broadly to a polypeptide that comprises, or alternatively consists of, an amino acid sequence of an anti-BCL-6 antibody described herein and an amino acid sequence of a heterologous polypeptide (e.g., a polypeptide unrelated to an antibody or antibody domain).

“Host cell,” as used herein, refers broadly to the particular subject cell transfected with a nucleic acid molecule and the progeny or potential progeny of such a cell. Progeny may not be identical to the parent cell transfected with the nucleic acid molecule due to mutations or environmental influences that may occur in succeeding generations or integration of the nucleic acid molecule into the host cell genome.

“Treatment” refers broadly to both therapeutic treatment and prophylactic or preventative measures. Those in need of treatment include those already with the disorder as well as those in which the disorder is to be prevented. As used herein, the term “treating,” refers broadly to treating a disease, arresting, or reducing the development of the disease or its clinical symptoms, and/or relieving the disease, causing regression of the disease or its clinical symptoms. Therapy encompasses prophylaxis, treatment, remedy, reduction, alleviation, and/or providing relief from a disease, signs, and/or symptoms of a disease. Therapy encompasses an alleviation of signs and/or symptoms in patients with ongoing disease signs and/or symptoms. Therapy also encompasses “prophylaxis”. The term “reduced”, for purpose of therapy, refers broadly to the clinical significant reduction in signs and/or symptoms. Therapy includes treating relapses or recurrent signs and/or symptoms. Therapy encompasses but is not limited to precluding the appearance of signs and/or symptoms anytime as well as reducing existing signs and/or symptoms and eliminating existing signs and/or symptoms. Therapy includes treating chronic disease (“maintenance”) and acute disease. For example, treatment includes treating or preventing relapses or the recurrence of signs and/or symptoms.

“Effective amount,” as used herein, refers broadly to the amount of a compound, antibody, antigen, or cells that, when administered to a patient for treating a disease, is sufficient to effect such treatment for the disease. The effective amount may be an amount effective for prophylaxis, and/or an amount effective for prevention. The effective amount may be an amount effective to reduce, an amount effective to prevent the incidence of signs/symptoms, to reduce the severity of the incidence of signs/symptoms, to eliminate the incidence of signs/symptoms, to slow the development of the incidence of signs/symptoms, to prevent the development of the incidence of signs/symptoms, and/or effect prophylaxis of the incidence of signs/symptoms. The “effective amount” may vary depending on the disease and its severity and the age, weight, medical history, susceptibility, and pre-existing conditions, of the patient to be treated. The term “effective amount” is synonymous with “therapeutically effective amount” for purposes of this invention.

“Mammal,” as used herein, refers broadly to any and all warm-blooded vertebrate animals of the class Mammalia, characterized by a covering of hair on the skin and, in the female, milk-producing mammary glands for nourishing the young. Mammals include, but are not limited to, humans, domestic and farm animals, and zoo, sports, or pet animals. Examples of mammals include but are not limited to alpacas, armadillos, capybaras, cats, camels, chimpanzees, chinchillas, cattle, dogs, gerbils, goats, gorillas, guinea pigs, hamsters, horses, humans, lemurs, llamas, mice, non-human primates, pigs, rats, sheep, shrews, squirrels, and tapirs. Mammals include but are not limited to bovine, canine, equine, feline, murine, ovine, porcine, primate, and rodent species. Mammal also includes any and all those listed on the Mammal Species of the World maintained by the National Museum of Natural History, Smithsonian Institution in Washington D.C. Similarly, the term “subject” or “patient” includes both human and veterinary subjects and/or patients.

“Substantially free,” as used herein, refers broadly to the presence of a specific component in an amount less than 1%, preferably less than 0.1% or 0.01%. More preferably, the term “substantially free” refers broadly to the presence of a specific component in an amount less than 0.001%. The amount may be expressed as w/w or w/v depending on the composition.

### Anti-BCL-6 Antibodies

The anti-BCL-6 antibodies and antigen binding fragments thereof and methods of use described herein may be used to block Bcl-6 for therapeutic purposes and overcome shortfalls of current angiogenesis inhibitor drugs.

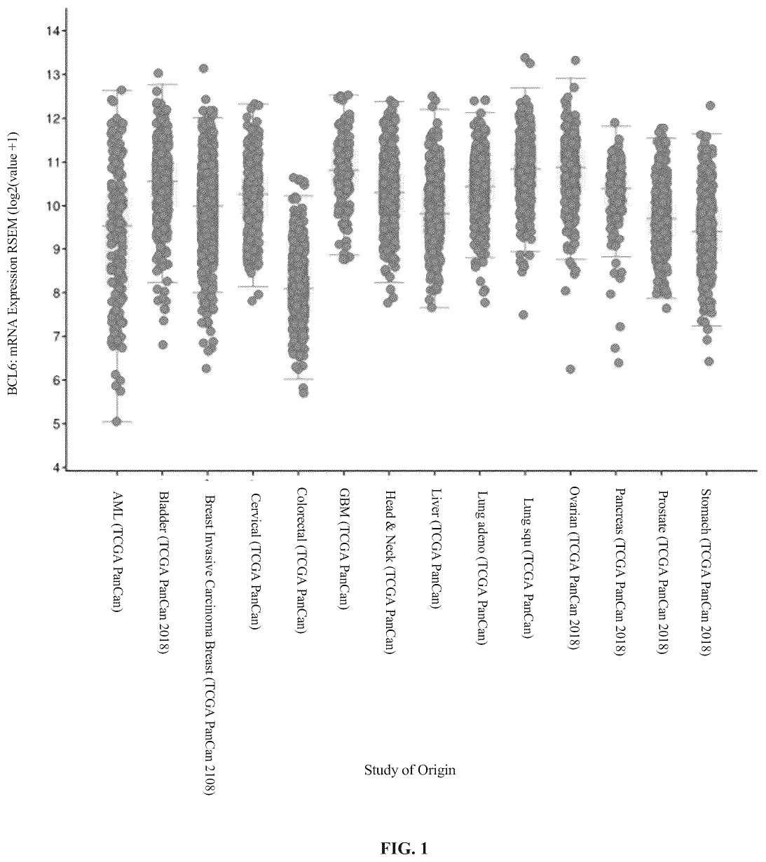

Bcl-6 is a transcriptional repressor protein and plays a critical role in regulating normal B cell differentiation. Dent et al. Critical Reviews in Oncology/Hematology (2002) 41(1): 1-9, incorporated herein by reference in its entirety. The molecular pathways by which Bcl-6 regulates B cell differentiation and also promotes the transformation of primary B cells are most likely related. The commonly accepted notion into Bcl-6's function is that it inhibits the terminal differentiation of activated B cells into plasma cells and that deregulation of Bcl-6 leads to an inhibition of terminal differentiation and continued proliferation. Niu et al. Hematol. Oncol. (2002). 20(4): 155-66, incorporated herein by reference in its entirety. Furthermore, Bcl-6 can promote the immortalization of primary B cells and can augment telomerase activity. Kusam et al. Immunol. Res. (2007) 37: 177-186, incorporated herein by reference in its entirety. Bcl-6 has recently been shown to act as an oncogene in several mouse model systems and have been demonstrated to serve a negative regulator of inflammation. Kusam et al. (2007). Attesting to its potential complex role in tumorigenesis, Bcl-6 is overexpressed in many cancers. See FIG. 1.

Human prostate cancer cells that over-expresses the Bcl-2 proto-oncogene show an increased angiogenic potential. Specifically, increased Bcl-2 expression enhanced the tumorigenic and angiogenic ability of human prostate cancer xenografts. Sakai et al. World J Urol. (2009) 27(5): 659-66, incorporated herein by reference in its entirety.

Human endothelial cells (HUVEC and HDMEC) cultured in conditioned media from Bcl-2 over expressing human prostate cancer cells resulted in increased rates of proliferation and the expression of key anti-apoptotic genes/proteins, possibly providing a survival advantage over endothelial cells grown in conditioned media from cancer cells with low Bcl-2 expression. Comparative genomic profiling of the treated and untreated endothelial cells revealed approximately 250 differentially expressed genes (p<0.001). After validation studies, Bcl-6, a well-known transcription factor of follicular helper T (Tfh) cells, stood out among several membrane bound proteins of interest (fold-change 2.85, p<0.000211).

Through a series of investigations with no direction as to which of many possible choices was likely to be successful, the inventors have shown that Bcl-6 influences neo-angiogenesis. The inventors surprisingly discovered that that the administration of a neutralizing antibody that targets Bcl-6 described herein resulted in the inhibition of endothelial cell proliferation and inhibition in endothelial tube formation (but not tube disruption) as well as the inhibition of cancer cell's ability to proliferate. Thus, therapeutic targeting of the Bcl-6 could offer a promising strategy to inhibit tumor angiogenesis and growth.

Given the key role of Bcl-6 in angiogenesis, it is possible to inhibit angiogenesis, thus treat diseases, by neutralizing Bcl-6 using the anti-BCL-6 antibodies and antigen-binding fragments described herein. The anti-BCL-6 antibodies and antigen-binding fragments described herein are capable of neutralizing Bcl-6 may be used in the treatment of many angiogenesis-dependent diseases, including but not limited to, cancer, preferably solid tumors.

Monoclonal anti-BCL-6 antibodies and antigen-binding fragments described herein have the ability to bind with high-specificity to BCL-6 (SEQ ID NO: 1 or 2), and may have a longer plasma half-life, lower toxicity, and/or fewer side effects as compared to other therapeutics. Furthermore, the fully human (or humanized) anti-BCL-6 antibodies and antigen-binding fragments described herein, can generally avoid immunogenicity issues.

Anti-BCL-6 antibodies may be used in the treatment of proliferative diseases, preferably cancer, optionally solid tumors. In some embodiments, the anti-BCL-6 antibodies described herein are used to treat as solid tumors, solid tumor metastases, angiofibromas, retrolental fibroplasia, hemangiomas, Kaposi's sarcoma and the like cancers which require neovascularization to support tumor growth. Other suitable tumors include melanoma, carcinoma, sarcoma, fibrosarcoma, glioma and astrocytoma.

The anti-BCL-6 antibodies described herein also find utility as reagents for detection and isolation of BCL-6, such as detection and/or quantification of BCL-6 expression in various cells and/or tissues. The anti-BCL-6 antibodies described herein can be used as BCL-6 antagonists.

The antibodies described herein immunospecifically may bind a BCL-6 polypeptide having the amino acid sequence of SEQ ID NO: 1, 2, or a combination thereof, or a polypeptide comprising a portion (e.g., a fragment) of the amino acid sequence of SEQ ID NO: 1, 2, or a combination thereof. The antibodies described herein including molecules comprising, or alternatively consisting of, antibody fragments or variants thereof that immunospecifically bind to the BCL-6 (e.g., a polypeptide comprising, or alternatively consisting of, amino acids 686 to 706 of BCL-6 (SEQ ID NO: 1). The antibodies and antigen binding fragments described herein preferably bind to an epitope consisting of 6 to 24 residues (SEQ ID NO: 2) within the BCL-6 protein (SEQ ID NO: 1).

Moreover, polypeptide fragments that may be bound by antibodies of the present invention, can be at least 10, 20, 30, 40, 50, 60, 70, 80, 90, 100, 110, 120, 130, 140, 150, 175 or 200 amino acids in length. In this context, “about” means the particularly recited ranges and ranges larger or smaller by several, a few, 5, 4, 3, 2 or 10 amino acid residues at either or both the amino- and carboxy-termini.

Additional embodiments described herein encompass antibodies that bind BCL-6 polypeptide fragments comprising, or alternatively consisting of, functional regions of polypeptides described herein, such as the Gamier-Robson alpha-regions, beta-regions, turn-regions, and coil-regions, Chou-Fasman alpha-regions, beta-regions, and coil-regions, Kyte-Doolittle hydrophilic regions and hydrophobic regions, Eisenberg alpha- and beta-amphipathic regions, Karplus-Schulz flexible regions, Emini surface-forming regions and Jameson-Wolf regions of high antigenic index. In a preferred embodiment, the polypeptide fragments bound by the antibodies described herein are antigenic (e.g., containing four or more contiguous amino acids having an antigenic index of greater than or equal to 1.5, as identified using the default parameters of the Jameson-Wolf program) of a complete (e.g., full-length) BCL-6 polypeptide (e.g., SEQ ID NO: 1 (full peptide) and 2 (epitope)).

The antibodies described herein may bind a polypeptide comprising, or alternatively consisting of, an epitope-bearing portion of a polypeptide described herein. The epitope of this polypeptide portion may be an immunogenic or antigenic epitope of a polypeptide described herein. For example, the epitope may comprise the amino acid sequence of SEQ ID NO: 2. An “immunogenic epitope” is a part of a protein that elicits an antibody response when the whole protein is the immunogen. On the other hand, a region of a protein molecule to which an antibody can bind is an “antigenic epitope.” The number of immunogenic epitopes of a protein generally is less than the number of antigenic epitopes. See, for instance, Geysen et al. Proc. Natl. Acad. Sci. USA 81:3998-4002 (1983), incorporated herein by reference in its entirety.

As to the selection of polypeptides bearing an antigenic epitope (e.g., that contain a region of a protein molecule to which an antibody can bind), it is well known in that art that relatively short synthetic peptides that mimic part of a protein sequence are routinely capable of eliciting an antiserum that reacts with the partially mimicked protein. See, for instance, Sutcliffe et al. (1983) Science 219: 660-666, incorporated herein by reference in its entirety. Peptides capable of eliciting protein-reactive sera are frequently represented in the primary sequence of a protein, can be characterized by a set of simple chemical rules, and are confined neither to immunodominant regions of intact proteins (e.g., immunogenic epitopes) nor to the amino or carboxyl terminals. Antigenic epitope-bearing peptides and polypeptides described herein are therefore useful to raise antibodies, including monoclonal antibodies, that bind specifically to a polypeptide described herein. See, for instance, Wilson et al. Cell 37: 767-778 (1984), incorporated herein by reference in its entirety, at 777.

The antibodies described herein may bind antigenic epitope-bearing peptides and polypeptides of BCL-6 and preferably contain a sequence of at least 4, at least 5, at least 6, at least 7, more preferably at least 8, at least 9, at least 10, at least 11, at least 12, at least 13, at least 14, at least 15, at least 20, at least 25, at least 30, at least 40, at least 50, and, most preferably, between about 15 to about 30 amino acids contained within the amino acid sequence of a BCL-6 polypeptide. Preferred polypeptides comprising immunogenic or antigenic epitopes are at least 10, 15, 20, 25, 30, 35, 40, 45, 50, 55, 60, 65, 70, 75, 80, 85, 90, 95, or 100 amino acid residues in length. Additional non-exclusive preferred antigenic epitopes include the antigenic epitopes disclosed herein, as well as portions thereof.

### Anti-BCL-6 Antibody Epitopes

Antibodies that bind polypeptides comprising, or alternatively consisting of, an epitope contained within the polypeptide having an amino acid sequence of SEQ ID NO: 1, 2, or a combination thereof, are described herein.

The antibodies and antigen binding fragments described herein preferably bind to an epitope consisting of 6 to 24 residues within the receptor binding domain of amino acids 686-706 of the BCL-6 (SEQ ID NO: 1), amino acid residues 686-706 (SEQ ID NO: 2).

The term “epitopes,” as used herein, refers broadly to portions of a polypeptide having antigenic or immunogenic activity in an animal, preferably a mammal, and most preferably in a human. In a preferred embodiment, the present invention encompasses antibodies that bind a polypeptide comprising an epitope. An “immunogenic epitope,” as used herein, is defined as a portion of a protein that elicits an antibody response in an animal, as determined by any method known in the art, for example, by the methods for generating antibodies described infra. See, for example, Geysen et al. Proc. Natl. Acad. Sci. USA 81: 3998-4002 (1983), incorporated herein by reference in its entirety. The term “antigenic epitope,” as used herein, is defined as a portion of a protein to which an antibody can immunospecifically bind its antigen as determined by any method well known in the art, for example, by the immunoassays described herein. Immunospecific binding excludes non-specific binding but does not necessarily exclude cross-reactivity with other antigens. Antigenic epitopes need not necessarily be immunogenic. Antigenic epitopes are useful, for example, to raise antibodies, including monoclonal antibodies, that specifically bind the epitope. Preferred antigenic epitopes include, but are not limited to, the antigenic epitopes disclosed herein, as well as any combination of two, three, four, five or more of these antigenic epitopes. Antigenic epitopes can be used as the target molecules in immunoassays. See, for instance, Wilson et al. Cell 37: 767-778 (1984); Sutcliffe et al. Science 219:660-666 (1983), each of which is incorporated herein by reference in its entirety.

Similarly, immunogenic epitopes can be used, for example, to induce antibodies according to methods well known in the art. See, for instance, Sutcliffe et al. supra; Wilson et al. supra; Chow et al. Proc. Natl. Acad. Sci. USA 82: 910-914; and Bittle et al. J. Gen. Virol. 66:2347-2354 (1985), each of which incorporated herein by reference in its entirety. Preferred immunogenic epitopes include the immunogenic epitopes disclosed herein, as well as any combination of two, three, four, five or more of these immunogenic epitopes. The polypeptides comprising one or more immunogenic epitopes of BCL-6 may be presented for eliciting an antibody response together with a carrier protein, such as an albumin, to an animal system (such as rabbit or mouse), or, if the polypeptide is of sufficient length (at least about 25 amino acids), the polypeptide may be presented without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting).

BCL-6 polypeptide fragments which function as epitopes may be produced by any conventional means. See, e.g., Houghten Proc. Natl. Acad. Sci. USA 82: 5131-5135 (1985) and U.S. Pat. No. 4,631,211, each of which is incorporated herein by reference in its entirety.

Epitope-bearing BCL-6 polypeptides (e.g., SEQ ID NO: 1 and 2) may be used to induce antibodies according to methods well known in the art including, but not limited to, in vivo immunization, in vitro immunization, and phage display methods. See, e.g., Sutcliffe et al. supra; Wilson et al. supra, and Bittle et al. J. Gen. Virol. 66: 2347-2354 (1985), each of which is incorporated herein by reference in its entirety. If in vivo immunization is used, animals may be immunized with free peptide; however, antipeptide antibody titer may be boosted by coupling the peptide to a macromolecular carrier, such as keyhole limpet hemocyanin (KLH) or tetanus toxoid. For instance, peptides containing cysteine residues may be coupled to a carrier using a linker such as maleimidobenzoyl-N-hydroxysuccinimide ester (MBS), while other peptides may be coupled to carriers using a more general linking agent such as glutaraldehyde. Animals such as rabbits, rats and mice are immunized with either free or carrier-coupled peptides, for instance, by intraperitoneal and/or intradermal injection of emulsions containing about 100 micrograms of peptide or carrier protein and Freund's adjuvant or any other adjuvant known for stimulating an immune response. Several booster injections may be needed, for instance, at intervals of about two weeks, to provide a useful titer of anti-peptide antibody that can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

### Anti-BCL-6 Antibody Fusion Proteins

The antibodies of the present invention may bind polypeptides comprising an immunogenic or antigenic epitope fused to other polypeptide sequences. For example, the BCL-6 polypeptides may be fused with the constant domain of immunoglobulins (IgA, IgE, IgG, IgM), or portions thereof (CH1, CH2, CH3, or any combination thereof and portions thereof), or albumin (including but not limited to recombinant human albumin or fragments or variants thereof (See, e.g., U.S. Pat. No. 5,876,969, EP Patent No. 0 413 622, and U.S. Pat. No. 5,766,883, each of which is incorporated herein by reference in its entirety), resulting in chimeric polypeptides. Such fusion proteins may facilitate purification and may increase half-life in vivo. This has been shown for chimeric proteins consisting of the first two domains of the human CD4-polypeptide and various domains of the constant regions of the heavy or light chains of mammalian immunoglobulins. See, e.g., EP 0394827; Traunecker et al. Nature 331: 84-86 (1988), each of which is incorporated herein by reference in its entirety. Enhanced delivery of an antigen across the epithelial barrier to the immune system has been demonstrated for antigens (e.g., insulin) conjugated to an FcRn binding partner such as IgG or Fe fragments (See, e.g., WO 96/22024 and WO 99/04813, each of which incorporated herein by reference in its entirety). IgG fusion proteins that have a disulfide-linked dimeric structure due to the IgG portion disulfide bonds have also been found to be more efficient in binding and neutralizing other molecules than monomeric polypeptides or fragments thereof alone. See, e.g., Fountoulakis et al. J. Biochem. 270:3958-3964 (1995), incorporated herein by reference in its entirety. Nucleic acids encoding the above epitopes can also be recombined with a gene of interest as an epitope tag (e.g., the hemagglutinin (“HA”) tag or flag tag) to aid in detection and purification of the expressed polypeptide. For example, a system described by Janknecht et al. allows for the ready purification of non-denatured fusion proteins expressed in human cell lines (Janknecht et al. (1991) Proc. Natl. Acad. Sci. USA 88: 8972-897 (incorporated herein by reference in its entirety)): In this system, the gene of interest is subcloned into a vaccinia recombination plasmid such that the open reading frame of the gene is translationally fused to an amino-terminal tag consisting of six histidine residues. The tag serves as a matrix-binding domain for the fusion protein. Extracts from cells infected with the recombinant vaccinia virus are loaded onto Ni nitriloacetic acid-agarose column and histidine-tagged proteins can be selectively eluted with imidazole-containing buffers.

The antibodies of the present invention may bind BCL-6 polypeptides and/or the epitope-bearing fragments thereof that are fused with a heterologous antigen (e.g., polypeptide, carbohydrate, phospholipid, or nucleic acid). In specific embodiments, the heterologous antigen is an immunogen.

### Anti-BCL-6 Antibody Specificity

The binding specificity of antibodies described herein to BCL-6 polypeptides (SEQ ID NOs: 1 and 2), or fragments or variants thereof can be determined by any suitable means. Examples of suitable assays to measure binding specificity include, but are not limited to, immunoprecipitation or in vitro binding assays, such as radioimmunoassay (RIA) or enzyme-linked immunoadsorbent assay (ELISA). Other means, such as, surface plasmon resonance may also be used.

The binding affinity of antibodies can, for example, be determined by the Scatchard analysis described by Frankel et al. Mol. Immunol. 16:101-106, 1979, incorporated herein by reference in its entirety. Binding affinity may be measured by an antigen/antibody dissociation rate. A high binding affinity may be measured by a competition radioimmunoassay. Binding affinity may be measured by ELISA. Antibody affinity may be measured by flow cytometry.

An antibody that “specifically binds” or “immunospecifically binds” an antigen (such as BCL-6 or fragments or variants thereof) is an antibody that binds the antigen with high affinity and does not significantly bind other unrelated antigens.

The antibodies described herein may bind a BCL-6 polypeptide or fragment thereof (such as soluble and/or cell-surface BCL-6) with a dissociation constant (K_{d}) of about 1 nM or less. In some embodiments, the antibodies bind a BCL-6 polypeptide or fragment thereof with a binding affinity of about 1 nM, about 0.9 nM, about 0.8 nM, about 0.7 nM, about 0.6 nM, about 0.5 nM, about 0.4 nM, about 0.3 nM, about 0.2 nM, about 0.15 nM, about 0.1 nM, about 0.05 nM, about 0.04 nM, about 0.03 nM, about 0.02 nM or about 0.01 nM.

Some embodiments described herein are directed to antibodies that bind polypeptides comprising, or alternatively consisting of, a polypeptide having an amino acid sequence least 80%, 85%, 90% identical and more preferably at least 95%, 96%, 97%, 98%, 99% or 100% identical to a BCL-6 polypeptide having the amino acid sequence at positions within SEQ ID NO: 1, 2, or a combination thereof.

Additional embodiments described herein are directed to antibodies that bind polypeptides comprising, or alternatively consisting of, a polypeptide having an amino acid sequence of about 90% to 99% sequence identity to a BCL-6 polypeptide having the amino acid sequence of SEQ ID NO: 1, 2, or a combination thereof. The anti-BCL-6 antibodies may selectively bind to a polypeptide having at least about 91%, 92%, 93%, 94%, 95%, 96%, 97%, 98%, 99%, or 100% sequence identity to a BCL-6 polypeptide having the amino acid sequence of SEQ ID NO: 1, 2, or a combination thereof.

Antibodies of the present invention may bind fragments, derivatives or analogs of the polypeptide of SEQ ID NO: 1, 2, or a combination thereof, such as (i) polypeptides in which one or more of the amino acid residues are substituted with a conserved or non-conserved amino acid residue (preferably a conserved amino acid residue) and such substituted amino acid residue may or may not be one encoded by the genetic code, or (ii) polypeptides in which one or more of the amino acid residues includes a substituent group, or (iii) polypeptides in which the polypeptide is fused with another compound, such as a compound to increase the half-life of the polypeptide (for example, polyethylene glycol), or (iv) polypeptides in which the additional amino acids are fused to the polypeptide, such as an IgG Fc fusion region peptide or leader or secretory sequence or a sequence which is employed for purification of the polypeptide or a proprotein sequence.

Amino acids in the BCL-6 polypeptides that are essential for function can be identified by methods known in the art, such as site-directed mutagenesis or alanine-scanning mutagenesis. Cunningham & Wells Science 244:1081-1085 (1989), incorporated herein by reference in its entirety. The latter procedure introduces single alanine mutations at every residue in the molecule. The resulting mutant molecules are then tested for functional activity, such ligand binding. Accordingly, antibodies of the present invention may bind amino acids in the BCL-6 polypeptides that are essential for function. In some embodiments, antibodies of the present invention bind amino acids in the BCL-6 polypeptides that are essential for angiogenesis. In some embodiments, antibodies of the present invention bind amino acids in the BCL-6 polypeptides that inhibit or reduce angiogenesis, e.g., neovascularization in solid tumors. Sites that are critical for ligand-receptor binding can also be determined by structural analysis such as crystallization, nuclear magnetic resonance or photoaffinity labelling. Smith et al. J. Mol. Biol. 224:899-904 (1992) and de Vos et al. Science 255:306-312 (1992), each of which is incorporated herein by reference in its entirety.

### Anti-BCL-6 Antibody Activity

The anti-BCL-6 antibodies described herein inhibit the activity of human B cell lymphoma 6 protein. Any convenient angiogenesis inhibition assay is suitable for use herein. Such assays are well known in the art. Generally, cells are seeded in attached cell culture plates, grown for 1 day, and then incubated for an additional day in the presence of a predetermined number of units of a selected BCL-6 plus various concentrations of the candidate anti-BCL-6 antibody. The candidate anti-BCL-6 antibody that inhibits the activity of BCL-6 will inhibit more angiogenesis activity than the baseline level of angiogenesis activity inhibition measured in the presence of an equivalent concentration of control antibody. Optionally, the candidate anti-BCL-6 antibody that inhibits the activity of BCL-6, will inhibit at least and/or about 30%, or at least and/or about 40%, or at least and/or about 50%, or at least and/or about 60%, or at least and/or about 70%, or at least and/or about 80%, or at least and/or about 90%, or at least and/or about 95%, or at least and/or about 96%, or at least and/or about 97%, or at least and/or about 98%, or at least and/or about 99%, or about 100% of the activity of the BCL-6 in an activity assay as compared to baseline activity measured in the presence of an equivalent concentration of control antibody. The candidate anti-BCL-6 antibody that does not inhibit the activity of BCL-6 will exhibit similar or approximately the same level of angiogenesis activity inhibition as a control antibody.

If an anti-BCL-6 monoclonal antibody that binds to a particular BCL-6 determinant(s) is desired, the candidate antibody can be screened for the presence or absence of differential affinity to wild type BCL-6 and to mutant BCL-6 that contains Ala substitution(s) at the determinant(s) of interest as described above. In one aspect, the candidate antibody can be tested for binding to wild type BCL-6 and mutant BCL-6 in an immunoprecipitation or immunoadsorption assay. For example, a capture ELISA can be used wherein plates are coated with a given concentration of wild type BCL-6 or an equal concentration of mutant BCL-6, the coated plates are contacted with equal concentrations of the candidate antibody, and the bound antibody is detected enzymatically, e.g., contacting the bound antibody with HRP-conjugated anti-Ig antibody and developing the HRP color reaction. The candidate antibody that binds to the particular BCL-6 determinant(s) of interest will exhibit binding activity with wild type BCL-6 that is greater than the candidate antibody's binding activity with the corresponding Ala-substituted BCL-6 mutant (i.e., a binding level with wild type BCL-6 that is above the background binding level with mutant BCL-6). Optionally, the candidate antibody that binds to the particular BCL-6 determinant(s) of interest will exhibit binding activity with the corresponding Ala-substituted BCL-6 mutant that is less than about 50%, or less than about 30%, or less than about 20%, or less than about 10%, or less than about 7%, or less than about 6%, or less than about 5%, or less than about 4%, or less than about 3%, or less than about 2%, or less than about 1%, or about 0% of the antibody's binding activity with wild type BCL-6, e.g., as determined by dividing the HRP color reaction optical density observed for capture ELISA with BCL-6 mutant adsorbent by the HRP color reaction optical density observed for capture ELISA with wild type BCL-6 adsorbent.

### BCL-6 Epitopes

Provided herein are antibodies that immunospecifically bind to BCL-6 with high affinity and neutralize BCL-6. These antibodies have been demonstrated, for example, to strongly bind to BCL-6.

The amino acid sequence of BCL-6 is shown below (underlined text comprises the epitope):

(SEQ ID NO: 1)
MASPADSCIQFTRHASDVLLNLNRLRSRDILTDVVIVVSREQFRAHKTV
LMACSGLFYSIFTDQLKCNLSVINLDPEINPEGFCILLDFMYTSRLNLR
EGNIMAVMATAMYLQMEHVVDTCRKFIKASEAEMVSAIKPPREEFLNSR
MLMPQDIMAYRGREVVENNLPLRSAPGCESRAFAPSLYSGLSTPPASYS
MYSHLPVSSLLFSDEEFRDVRMPVANPFPKERALPCDSARPVPGEYSRP
TLEVSPNVCHSNIYSPKETIPEEARSDMHYSVAEGLKPAAPSARNAPYF
PCDKASKEEERPSSEDEIALHFEPPNAPLNRKGLVSPQSPQKSDCQPNS
PTESCSSKNACILQASGSPPAKSPTDPKACNWKKYKFIVLNSLNQNAKP
EGPEQAELGRLSPRAYTAPPACQPPMEPENLDLQSPTKLSASGEDSTIP
QASRLNNIVNRSMTGSPRSSSESHSPLYMHPPKCTSCGSQSPQHAEMCL
HTAGPTFPEEMGETQSEYSDSSCENGAFFCNECDCRFSEEASLKRHTLQ
THSDKPYKCDRCQASFRYKGNLASHKTVHTGEKPYRCNICGAQFNRPAN
LKTHTRIHSGEKPYKCETCGARFVQVAHLRAHVLIHTGEKPYPCEICGT
RFRHLQTLKSHLRIHTGEKPYHCEKCNLHFRHKSQLRLHLRQKHGAITN
TKVQYRVSATDLPPELPKAC

The antibody or fragment thereof described herein may selectively bind the BCL-6 amino acid sequence of SEQ ID NO: 1.

The antibody or fragment thereof described herein may selectively bind a BCL-6 epitope comprising the amino acid sequence of SEQ ID NO: 2 (TKVQYRVSATDLPPELPKAC).

### Anti-BCL-6 Antibodies and Antigen-Binding Fragments Thereof

Provided herein are antibodies and/or antigen binding fragments thereof that immunospecifically bind to BCL-6 with high affinity and neutralize BCL-6. These antibodies have been demonstrated, for example, to strongly bind to BCL-6.

The antibodies and/or antigen binding fragments thereof may be isolated, recombinant, and/or engineered. The antibodies and/or antigen binding fragments thereof monoclonal or polyclonal. The antibodies and/or antigen binding fragments thereof may be humanized or chimeric.

The recombinant and/or isolated antibody or antigen binding fragment thereof may specifically bind to a BCL-6 having at least about 80%, at least about 85%, at least about 90%, at least about 91%, at least about 92%, at least about 93%, at least about 94%, at least about 95%, at least about 96%, at least about 97%, at least about 98%, or at least about 99% sequence homology to an amino acid sequence comprising the amino acid sequence of SEQ ID NO: 1.

The recombinant and/or isolated antibody or antigen binding fragment thereof may specifically bind to an epitope on BCL-6 having at least about 80%, at least about 85%, at least about 90%, at least about 91%, at least about 92%, at least about 93%, at least about 94%, at least about 95%, at least about 96%, at least about 97%, at least about 98%, or at least about 99% sequence homology to an amino acid sequence comprising the amino acid sequence of SEQ ID NO: 2.

In an embodiment, the recombinant and/or isolated antibody or an antigen binding fragment thereof that specifically binds a human B cell lymphoma 6 protein (BCL-6) polypeptide comprises: (a) a heavy chain variable domain comprising a complementarity-determining region (CDR) 1 comprising the amino acid sequence of SEQ ID NO: 5 optionally comprising one or more amino acid mutations, substitutions, or deletions that do not diminish the binding ability of isolated antibody or an antigen binding fragment thereof, a CDR 2 comprising the amino acid sequence of SEQ ID NO: 6 optionally comprising one or more amino acid mutations, substitutions, or deletions that do not diminish the binding ability of isolated antibody or an antigen binding fragment thereof, and a CDR 3 comprising the amino acid sequence of SEQ ID NO: 7 optionally comprising one or more amino acid mutations, substitutions, or deletions that do not diminish the binding ability of isolated antibody or an antigen binding fragment thereof, and (b) a light chain variable domain comprising a CDR 1 comprising the amino acid sequence of SEQ ID NO: 12 optionally comprising one or more amino acid mutations, substitutions, or deletions that do not diminish the binding ability of isolated antibody or an antigen binding fragment thereof, a CDR 2 comprising the amino acid sequence of SEQ ID NO: 13 optionally comprising one or more amino acid mutations, substitutions, or deletions that do not diminish the binding ability of isolated antibody or an antigen binding fragment thereof, and a CDR 3 optionally comprising one or more amino acid mutations, substitutions, or deletions that do not diminish the binding ability of isolated antibody or an antigen binding fragment thereof.

In an embodiment, the recombinant and/or isolated antibody or an antigen binding fragment thereof that specifically binds a human B cell lymphoma 6 protein (BCL-6) polypeptide comprises: (a) a heavy chain variable domain comprising a complementarity-determining region (CDR) 1 comprising the amino acid sequence of SEQ ID NO: 5 comprising at most one conservative substitution, a CDR 2 comprising the amino acid sequence of SEQ ID NO: 6 comprising at most one conservative substitution, and a CDR 3 comprising the amino acid sequence of SEQ ID NO: 7 comprising at most one conservative substitution; and (b) a light chain variable domain comprising a CDR 1 comprising the amino acid sequence of SEQ ID NO: 12 comprising at most one conservative substitution, a CDR 2 comprising the amino acid sequence of SEQ ID NO: 13 comprising at most one conservative substitution, and a CDR 3 comprising the amino acid sequence of SEQ ID NO: 14 comprising at most one conservative substitution.

In an embodiment, (a) the heavy chain variable domain further comprises (i) a framework region (FR) 1 comprising the amino acid sequence of SEQ ID NO: 8 or an amino acid sequence at least about 85%, at least about 90%, at least about 91%, at least about 92%, at least about 93%, at least about 94%, at least about 95%, at least about 96%, at least about 97%, at least about 98%, or at least about 99% identical thereto, (ii) a FR 2 comprising the amino acid sequence of SEQ ID NO: 9 or an amino acid sequence at least about 85%, at least about 90%, at least about 91%, at least about 92%, at least about 93%, at least about 94%, at least about 95%, at least about 96%, at least about 97%, at least about 98%, or at least about 99% identical thereto, (iii) a FR 3 comprising the amino acid sequence of SEQ ID NO: 10 or an amino acid sequence at least about 85%, at least about 90%, at least about 91%, at least about 92%, at least about 93%, at least about 94%, at least about 95%, at least about 96%, at least about 97%, at least about 98%, or at least about 99% identical thereto, (iv) a FR 4 comprising the amino acid sequence of SEQ ID NO: 11 or an amino acid sequence at least about 85%, at least about 90%, at least about 91%, at least about 92%, at least about 93%, at least about 94%, at least about 95%, at least about 96%, at least about 97%, at least about 98%, or at least about 99% identical thereto; or (v) any combination of (i)-(iv); (b) the light chain variable domain further comprises (i) a FR 1 comprising the amino acid sequence of SEQ ID NO: 15 or an amino acid sequence at least about 85%, at least about 90%, at least about 91%, at least about 92%, at least about 93%, at least about 94%, at least about 95%, at least about 96%, at least about 97%, at least about 98%, or at least about 99% identical thereto, or a FR 1 comprising the amino acid sequence of SEQ ID NO: 23 or an amino acid sequence at least about 85%, at least about 90%, at least about 91%, at least about 92%, at least about 93%, at least about 94%, at least about 95%, at least about 96%, at least about 97%, at least about 98%, or at least about 99% identical thereto, (ii) a FR 2 comprising the amino acid sequence of SEQ ID NO: 16 or an amino acid sequence at least about 85%, at least about 90%, at least about 91%, at least about 92%, at least about 93%, at least about 94%, at least about 95%, at least about 96%, at least about 97%, at least about 98%, or at least about 99% identical thereto, (iii) a FR 3 comprising the amino acid sequence of SEQ ID NO: 17 or an amino acid sequence at least about 85%, at least about 90%, at least about 91%, at least about 92%, at least about 93%, at least about 94%, at least about 95%, at least about 96%, at least about 97%, at least about 98%, or at least about 99% identical thereto, (iv) a FR 4 comprising the amino acid sequence of SEQ ID NO: 18 or an amino acid sequence at least about 85%, at least about 90%, at least about 91%, at least about 92%, at least about 93%, at least about 94%, at least about 95%, at least about 96%, at least about 97%, at least about 98%, or at least about 99% identical thereto; or (v) any combination of (i)-(iv); or (c) a combination of (a) and (b).

In an embodiment, (a) the heavy chain variable domain further comprises (i) a framework region (FR) 1 comprising the amino acid sequence of SEQ ID NO: 8 optionally comprising one or more amino acid mutations, substitutions, or deletions that do not diminish the binding ability of isolated antibody or an antigen binding fragment thereof, (ii) a FR 2 comprising the amino acid sequence of SEQ ID NO: 9 optionally comprising one or more amino acid mutations, substitutions, or deletions that do not diminish the binding ability of isolated antibody or an antigen binding fragment thereof, (iii) a FR 3 comprising the amino acid sequence of SEQ ID NO: 10 optionally comprising one or more amino acid mutations, substitutions, or deletions that do not diminish the binding ability of isolated antibody or an antigen binding fragment thereof, (iv) a FR 4 comprising the amino acid sequence of SEQ ID NO: 11 optionally comprising one or more amino acid mutations, substitutions, or deletions that do not diminish the binding ability of isolated antibody or an antigen binding fragment thereof, or (v) any combination of (i)-(iv); (b) the light chain variable domain further comprises (i) a FR 1 comprising the amino acid sequence of SEQ ID NO: 15 optionally comprising one or more amino acid mutations, substitutions, or deletions that do not diminish the binding ability of isolated antibody or an antigen binding fragment thereof, or a FR 1 comprising the amino acid sequence of SEQ ID NO: 23 optionally comprising one or more amino acid mutations, substitutions, or deletions that do not diminish the binding ability of isolated antibody or an antigen binding fragment thereof, (ii) a FR 2 comprising the amino acid sequence of SEQ ID NO: 16 optionally comprising one or more amino acid mutations, substitutions, or deletions that do not diminish the binding ability of isolated antibody or an antigen binding fragment thereof, (iii) a FR 3 comprising the amino acid sequence of SEQ ID NO: 17 optionally comprising one or more amino acid mutations, substitutions, or deletions that do not diminish the binding ability of isolated antibody or an antigen binding fragment thereof, (iv) a FR 4 comprising the amino acid sequence of SEQ ID NO: 18 optionally comprising one or more amino acid mutations, substitutions, or deletions that do not diminish the binding ability of isolated antibody or an antigen binding fragment thereof; or (v) any combination of (i)-(iv); or (c) a combination of (a) and (b).

In an embodiment, (a) the heavy chain variable domain further comprises (i) a framework region (FR) 1 comprising the amino acid sequence of SEQ ID NO: 8 comprising at most one or two amino acid substitutions, (ii) a FR 2 comprising the amino acid sequence of SEQ ID NO: 9 comprising at most one or two amino acid substitutions, (iii) a FR 3 comprising the amino acid sequence of SEQ ID NO: 10 comprising at most one or two amino acid substitutions, (iv) a FR 4 comprising the amino acid sequence of SEQ ID NO: 11 comprising at most one or two amino acid substitutions; or (v) any combination of (i)-(iv); (b) the light chain variable domain further comprises (i) a FR 1 comprising the amino acid sequence of SEQ ID NO: 15 comprising at most one or two amino acid substitutions, a FR 1 comprising the amino acid sequence of SEQ ID NO: 23 comprising at most one or two amino acid substitutions, (ii) a FR 2 comprising the amino acid sequence of SEQ ID NO: 16 comprising at most one or two amino acid substitutions, (iii) a FR 3 comprising the amino acid sequence of SEQ ID NO: 17 comprising at most one or two amino acid substitutions, (iv) a FR 4 comprising the amino acid sequence of SEQ ID NO: 18 comprising at most one or two amino acid substitutions; or (v) any combination of (i)-(iv); or (c) a combination of (a) and (b).

In an embodiment, (a) the heavy chain variable domain further comprises (i) a framework region (FR) 1 comprising the amino acid sequence of SEQ ID NO: 8 comprising at most one or two conservative amino acid substitutions, (ii) a FR 2 comprising the amino acid sequence of SEQ ID NO: 9 comprising at most one or two conservative amino acid substitutions, (iii) a FR 3 comprising the amino acid sequence of SEQ ID NO: 10 comprising at most one or two conservative amino acid substitutions, (iv) a FR 4 comprising the amino acid sequence of SEQ ID NO: 11 comprising at most one or two conservative amino acid substitutions; or (v) any combination of (i)-(iv); (b) wherein light chain variable domain further comprises (i) a FR 1 comprising the amino acid sequence of SEQ ID NO: 15 comprising at most one or two conservative amino acid substitutions, (ii) a FR 2 comprising the amino acid sequence of SEQ ID NO: 16 comprising at most one or two conservative amino acid substitutions, (iii) a FR 3 comprising the amino acid sequence of SEQ ID NO: 17 comprising at most one or two conservative amino acid substitutions, (iv) a FR 4 comprising the amino acid sequence of SEQ ID NO: 18 comprising at most one or two conservative amino acid substitutions; or (v) any combination of (i)-(iv); or (c) a combination of (a) and (b).

In an embodiment, a recombinant and/or isolated antibody or an antigen binding fragment thereof that specifically binds a human B cell lymphoma 6 protein (BCL-6) polypeptide comprises a variable chain comprising the structure, from amino terminus to carboxy terminus:
FR1-CDR1-FR2-CDR2-FR3-CDR3-FR4.

In an embodiment, a recombinant and/or isolated antibody or an antigen binding fragment thereof that specifically binds a human B cell lymphoma 6 protein (BCL-6) polypeptide comprises a variable heavy (H) chain comprising the structure, from amino terminus to carboxy terminus:
HFR1-HCDR1-HFR2-HCDR2-HFR3-HCDR3-HFR4.

In an embodiment, a recombinant and/or isolated antibody or an antigen binding fragment thereof that specifically binds a human B cell lymphoma 6 protein (BCL-6) polypeptide comprises a variable light (L) chain comprising the structure, from amino terminus to carboxy terminus:
LFR1-LCDR1-LFR2-LCDR2-LFR3-LCDR3-LFR4.

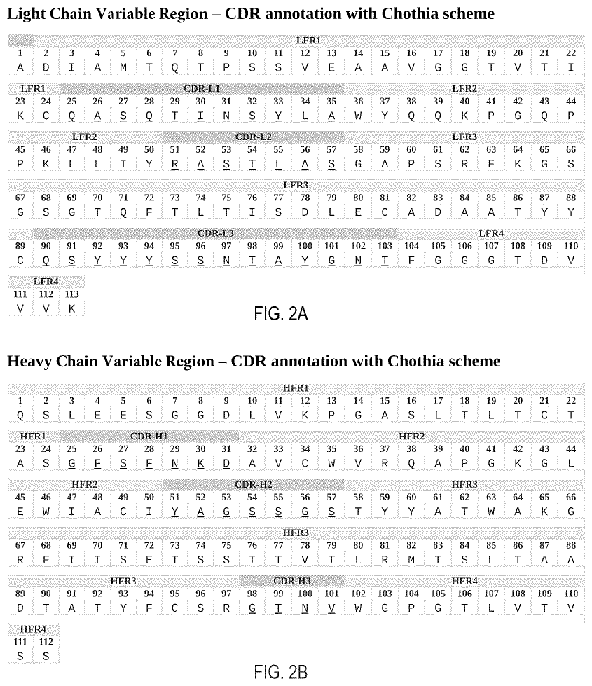

In an embodiment, a recombinant and/or isolated antibody or an antigen binding fragment thereof that specifically binds a human B cell lymphoma 6 protein (BCL-6) polypeptide comprises the CDRs and FRs set forth in FIG. 2, in the order set forth therein. In FIG. 2, the CDRs are underlined, and annotated according to the scheme set forth in Al-Lazikani B, Lesk AM, Chothia C (1997), “Standard conformations for the canonical structures of immunoglobulins”, Journal of Molecular Biology, 273 (4): 927-48, incorporated by reference herein in its entirety.

In an embodiment, a recombinant and/or isolated antibody or an antigen binding fragment thereof that specifically binds a human B cell lymphoma 6 protein (BCL-6) polypeptide comprises: (a) a heavy chain variable domain comprising the amino acid sequence of SEQ ID NO: 3 or an amino acid sequence at least about 85%, at least about 90%, at least about 91%, at least about 92%, at least about 93%, at least about 94%, at least about 95%, at least about 96%, at least about 97%, at least about 98%, or at least about 99% identical thereto; and (b) a light chain variable domain comprising the amino acid sequence of SEQ ID NO: 4 or an amino acid sequence at least about 85%, at least about 90%, at least about 91%, at least about 92%, at least about 93%, at least about 94%, at least about 95%, at least about 96%, at least about 97%, at least about 98%, or at least about 99% identical thereto.

In an embodiment, the recombinant and/or isolated antibody or an antigen binding fragment further comprises: (a) a heavy chain constant domain or portion thereof, (b) a light chain constant domain or portion thereof, or (c) a combination of (a) and (b). In an embodiment, (a) the heavy chain constant domain or portion thereof is or is derived from an alpha (α), delta (δ), epsilon (ε), gamma (γ), or mu (μ) constant domain; (b) the light chain constant domain or portion thereof is or is derived from a kappa (κ) or lambda (λ) constant domain; or (c) a combination of (a) and (b). In an embodiment, (a) the heavy chain constant domain is or is derived from a gamma (γ) constant domain; (b) the light chain constant domain is or is derived from a kappa (κ) constant domain; or (c) a combination of (a) and (b).

In an embodiment, (a) the heavy chain constant domain comprises the amino acid sequence of SEQ ID NO: 19 or an amino acid sequence at least about 85%, at least about 90%, at least about 91%, at least about 92%, at least about 93%, at least about 94%, at least about 95%, at least about 96%, at least about 97%, at least about 98%, or at least about 99% identical thereto; (b) the light chain constant domain comprises the amino acid sequence of SEQ ID NO: 20 or an amino acid sequence at least about 85%, at least about 90%, at least about 91%, at least about 92%, at least about 93%, at least about 94%, at least about 95%, at least about 96%, at least about 97%, at least about 98%, or at least about 99% identical thereto; or (c) a combination of (a) and (b).

In an embodiment, the recombinant and/or isolated antibody that specifically binds a human B cell lymphoma 6 protein (BCL-6) polypeptide comprises: (i) a heavy chain comprising the amino acid sequence of SEQ ID NO: 21 or an amino acid sequence at least about 85%, at least about 90%, at least about 91%, at least about 92%, at least about 93%, at least about 94%, at least about 95%, at least about 96%, at least about 97%, at least about 98%, or at least about 99% identical thereto; and (ii) a light chain comprising the amino acid sequence of SEQ ID NO: 22 or an amino acid sequence at least about 85%, at least about 90%, at least about 91%, at least about 92%, at least about 93%, at least about 94%, at least about 95%, at least about 96%, at least about 97%, at least about 98%, or at least about 99% identical thereto.

### Anti-BCL-6 Antibody Variants

The antibodies described herein may be from any animal origin, including birds and mammals. Preferably, the antibodies are human, murine (e.g., mouse and rat), donkey, sheep, rabbit, goat, guinea pig, camel, horse, or chicken. In some embodiments, the antibodies are human antibodies. As used herein, “human” antibodies include antibodies having the amino acid sequence of a human immunoglobulin and include antibodies isolated from human immunoglobulin libraries and xenomice or other organisms that have been genetically engineered to produce human antibodies. For a detailed discussion of a few of the technologies for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598; and Lenberg and Huszar Int. Rev. Immunol. 13:65-93 (1995), each of which is incorporated herein by reference in its entirety. Human antibodies or “humanized” chimeric monoclonal antibodies can be produced using techniques described herein or otherwise known in the art. For example, methods for producing chimeric antibodies are known in the art. See, for review the following references each of which is hereby incorporated herein in its entirety: Morrison Science 229:1202 (1985); Oi et al. BioTechniques 4:214 (1986); Cabilly et al. U.S. Pat. No. 4,816,567; Taniguchi et al. EP 171496; Morrison et al. EP 173494; Neuberger et al. WO 8601533; Robinson et al. WO 8702671; Boulianne et al. Nature 312:643 (1984); Neuberger et al. Nature 314:268 (1985).

### Chimeric Anti-BCL-6 Antibodies

A chimeric antibody is a molecule in which different portions of the antibody are derived from different immunoglobulin molecules such as antibodies having a variable region derived from a human antibody and a non-human immunoglobulin constant region. Methods for producing chimeric antibodies are known in the art. See e.g., Morrison, Science 229:1202 (1985); Oi et al. BioTechniques 4:214 (1986); Gillies et al. J. Immunol. Methods 125:191-202 (1989); U.S. Pat. Nos. 5,807,715; 4,816,567; and 4,816,397, each which is incorporated herein by reference in its entirety. Chimeric antibodies comprising one or more CDRs from human species and framework regions from a non-human immunoglobulin molecule (e.g., framework regions from a canine or feline immunoglobulin molecule) can be produced using a variety of techniques known in the art including, for example, CDR-grafting (EP 239,400; PCT publication WO 91/09967; U.S. Pat. Nos. 5,225,539; 5,530,101; and 5,585,089, each of which is incorporated herein by reference in its entirety), veneering or resurfacing (EP 592,106; EP 519,596; Padlan, Molecular Immunology 28(4/5):489-498 (1991); Studnicka et al. Protein Engineering 7(6):805-814 (1994); Roguska et al. PNAS 91:969-973 (1994), each of which is incorporated herein by reference in its entirety), and chain shuffling (U.S. Pat. No. 5,565,332, incorporated herein by reference in its entirety). Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. See, e.g., U.S. Pat. No. 5,585,089; Riechmann et al. Nature 332:323 (1988), each of which is incorporated herein by reference in its entireties.

### Monoclonal Anti-BCL-6 Antibodies

The monoclonal antibodies disclosed herein can be of any isotype. The monoclonal antibody can be, for example, IgA, IgD, IgE, IgM, or an IgG antibody, such as IgG1 or an IgG2. The class of an antibody that immunospecifically binds BCL-6 can be switched with another (for example, IgG can be switched to IgM), according to well-known procedures. Class switching can also be used to convert one IgG subclass to another, such as from IgG1 to IgG2. The monoclonal antibodies described herein are IgA, including serum IgA and secretory IgA (sIgA), more preferably secretory IgA (sIgA).

The antibodies of the present invention may be monovalent, bivalent, trivalent or multivalent. For example, monovalent scFvs can be multimerized either chemically or by association with another protein or substance. An scFv that is fused to a hexahistidine tag or a Flag tag can be multimerized using Ni-NTA agarose (Qiagen) or using anti-Flag antibodies (Stratagene, Inc.).

The antibodies of the present invention may be monospecific, bispecific, trispecific or of greater multispecificity. Multispecific antibodies may be specific for different epitopes of a BCL-6 polypeptide, or fragment thereof, or may be specific for both a BCL-6 polypeptide, or fragment thereof, and a heterologous epitope, such as a heterologous polypeptide or solid support material. See, e.g., PCT publications WO 93/17715; WO 92/08802; WO 91/00360; WO 92/05793; Tutt, et al. J. Immunol. 147:60-69 (1991); U.S. Pat. Nos. 4,474,893; 4,714,681; 4,925,648; 5,573,920; 5,601,819; Kostelny et al. J. Immunol. 148:1547-1553 (1992), each of which is incorporated herein by reference in its entirety.

### Anti-BCL-6 Antibody Cross-Reactivity

Antibodies of the present invention may also be described or specified in terms of their cross-reactivity. Antibodies that do not bind any other analog, ortholog, or homolog of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide of the present invention are also included in the present invention.

Antibodies of the present invention may cross-react with murine, rat and/or rabbit homologs of human proteins and the corresponding epitopes thereof. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are also included in the present invention. In a specific embodiment, the above-described cross-reactivity is with respect to any single specific antigenic or immunogenic polypeptide, or combination(s) of 2, 3, 4, 5, or more of the specific antigenic and/or immunogenic polypeptides disclosed herein. Further included in the present invention are antibodies which bind polypeptides encoded by polynucleotides which hybridize to a polynucleotide of the present invention under hybridization conditions (as described herein).

In some embodiments, the antibodies of the present invention (including molecules comprising, or alternatively consisting of, antibody fragments or variants thereof), immunospecifically bind to BCL-6 and do not cross-react with any other antigens.

### Anti-BCL-6 Variants and Derivatives

The present invention also provides antibodies that comprise, or alternatively consist of, variants (including derivatives) of the VH domains, VH CDRs, VL domains, and VL CDRs described herein, which antibodies immunospecifically bind to BCL-6. Standard techniques known to those of skill in the art can be used to introduce mutations in the nucleotide sequence encoding a molecule described herein, including, for example, site-directed mutagenesis and PCR-mediated mutagenesis which result in amino acid substitutions. Preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference VH domain, VHCDR1, VHCDR2, VHCDR3, VL domain, VLCDR1, VLCDR2, or VLCDR3. In some embodiments, the variants have conservative amino acid substitutions at one or more predicted non-essential amino acid residues. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind BCL-6). Following mutagenesis, the encoded protein may routinely be expressed and the functional and/or biological activity of the encoded protein, (e.g., ability to immunospecifically bind BCL-6) can be determined using techniques described herein or by routinely modifying techniques known in the art.

The antibodies described herein include derivatives (e.g., variants) that are modified, e.g., by the covalent attachment of any type of molecule to the antibody such that covalent attachment does not affect the ability of the antibody to immunospecifically bind to BCL-6. For example, but not by way of limitation, derivatives described herein include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

Also, glycosylation of the antibody of the present disclosure may be altered for adjusting the affinity of an antibody for a target antigen. Such alteration can be achieved by, for example, changing one or more glycosylation sites within the antibody sequence. More specifically, for example, one or more amino acid substitutions are introduced into an amino acid sequence composing one or more glycosylation sites within FR so as to remove the glycosylation sites, so that deglycosylation can be achieved at the sites. Such deglycosylation may be effective for increasing the affinity of an antibody for an antigen (U.S. Pat. Nos. 5,714,350 and 6,350,861, each of which is incorporated herein in its entirety). Optionally, the antibody carries a further effector function such as an immune stimulating domain or toxin.

In an embodiment, an antibody or antigen-binding fragment thereof described herein is labeled with a radionucleotide, a fluorescent dye, a fluorescent protein, an enzyme, biotin and/or (strept)avidin. In an embodiment, the radionucleotide is ⁶⁴Cu, ¹¹¹In, ⁹⁹Tc, ¹⁴C, ¹³¹I, ³H, ³²P or ³⁵S. In an aspect, the fluorescent dye is fluorescein isothiocyanate (FITC), rhodamine, Texas red, Cy3, or Cy5. In an embodiment, the fluorescent protein is phycoerythrin (PE), allophycocyanin (APC), or green fluorescent protein (GFP). An enzyme described herein may be selected from the group consisting of horseradish peroxidase, alkaline phosphatase, or glucose oxidase.

### Anti-BCL-6 Antibodies Sequences and Structure

In a specific embodiment, an antibody described herein (including a molecule comprising, or alternatively consisting of, an antibody fragment or variant thereof), that immunospecifically binds BCL-6, comprises, or alternatively consists of, an amino acid sequence encoded by a nucleotide sequence that hybridizes to a nucleotide sequence that is complementary to that encoding one of the VH or VL domains disclosed herein under stringent conditions, e.g., hybridization to filter-bound DNA in 6× sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art (see, for example, Ausubel, F. M. et al. eds., 1989, Current Protocols in Molecular Biology, Vol. I, Green Publishing Associates, Inc. and John Wiley & Sons, Inc., New York at pages 6.3.1-6.3.6 and 2.10.3, incorporated herein by reference in its entirety). In another embodiment, an antibody described herein that immunospecifically binds to BCL-6, comprises, or alternatively consists of, an amino acid sequence encoded by a nucleotide sequence that hybridizes to a nucleotide sequence that is complementary to that encoding one of heavy chain constant region and/or light chain constant region disclosed herein under stringent conditions, e.g., hybridization under conditions as described above, or under other stringent hybridization conditions which are known to those of skill in the art. Nucleic acid molecules encoding these antibodies are also encompassed by the invention.

The invention also encompasses antibodies (including molecules comprising, or alternatively consisting of, antibody fragments or variants thereof) that have one or more of the same biological characteristics as one or more of the antibodies described herein. By “biological characteristics” is meant, the in vitro or in vivo activities or properties of the antibodies, such as, for example, the ability to bind to BCL-6 and/or an antigenic and/or epitope region of BCL-6), the ability to substantially block BCL-6 activity. Optionally, the antibodies described herein will bind to the same epitope as at least one of the antibodies specifically referred to herein. Such epitope binding can be routinely determined using assays known in the art.

In other embodiments, the invention provides antibodies that competitively inhibit binding of an antibody comprising a fragment (e.g., VH domain, VL domain, VHCDR1, VHCDR2, VHCDR3, VLCDR1, VLCDR2, or VLCDR3) described herein or variant thereof to a BCL-6 polypeptide. In preferred embodiments, the invention provides antibodies which reduce the binding of an antibody comprising a fragment (e.g., VH domain, VL domain, VHCDR1, VHCDR2, VHCDR3, VLCDR1, VLCDR2, or VLCDR3) described herein or variant thereof to a BCL-6 polypeptide by between 1% and 10% in a competitive inhibition assay.

In preferred embodiments, the invention provides antibodies which reduce the binding of an antibody comprising a fragment (e.g., VH domain, VL domain, VHCDR1, VHCDR2, VHCDR3, VLCDR1, VLCDR2, or VLCDR3) described herein or variant thereof to a BCL-6 polypeptide by at least 10% and up to 20% in a competitive inhibition assay.

In preferred embodiments, the invention provides antibodies which reduce the binding of an antibody comprising a fragment (e.g., VH domain, VL domain, VHCDR1, VHCDR2, VHCDR3, VLCDR1, VLCDR2, or VLCDR3) described herein or variant thereof to a BCL-6 polypeptide by at least 20% and up to 30% in a competitive inhibition assay.

In preferred embodiments, the invention provides antibodies which reduce the binding of an antibody comprising a fragment (e.g., VH domain, VL domain, VHCDR1, VHCDR2, VHCDR3, VLCDR1, VLCDR2, or VLCDR3) described herein or variant thereof to a BCL-6 polypeptide by at least 30% and up to 40% in a competitive inhibition assay.

In preferred embodiments, the invention provides antibodies which reduce the binding of an antibody comprising a fragment (e.g., VH domain, VL domain, VHCDR1, VHCDR2, VHCDR3, VLCDR1, VLCDR2, or VLCDR3) described herein or variant thereof to a BCL-6 polypeptide by at least 40% and up to 50% in a competitive inhibition assay.

In preferred embodiments, the invention provides antibodies which reduce the binding of an antibody comprising a fragment (e.g., VH domain, VL domain, VHCDR1, VHCDR2, VHCDR3, VLCDR1, VLCDR2, or VLCDR3) described herein or variant thereof to a BCL-6 polypeptide by at least 50% and up to 60% in a competitive inhibition assay.

In preferred embodiments, the invention provides antibodies which reduce the binding of an antibody comprising a fragment (e.g., VH domain, VL domain, VHCDR1, VHCDR2, VHCDR3, VLCDR1, VLCDR2, or VLCDR3) described herein or variant thereof to a BCL-6 polypeptide by at least 60% and up to 70% in a competitive inhibition assay.

In preferred embodiments, the invention provides antibodies which reduce the binding of an antibody comprising a fragment (e.g., VH domain, VL domain, VHCDR1, VHCDR2, VHCDR3, VLCDR1, VLCDR2, or VLCDR3) described herein or variant thereof to a BCL-6 polypeptide by at least 70% and up to 80% in a competitive inhibition assay.

In preferred embodiments, the invention provides antibodies which reduce the binding of an antibody comprising a fragment (e.g., VH domain, VL domain, VHCDR1, VHCDR2, VHCDR3, VLCDR1, VLCDR2, or VLCDR3) described herein or variant thereof to a BCL-6 polypeptide by at least 80% and up to 90% in a competitive inhibition assay.

In preferred embodiments, the invention provides antibodies which reduce the binding of an antibody comprising a fragment (e.g., VH domain, VL domain, VHCDR1, VHCDR2, VHCDR3, VLCDR1, VLCDR2, or VLCDR3) described herein or variant thereof to a BCL-6 polypeptide by at least 90% and up to 100% in a competitive inhibition assay.

The present invention also provides for mixtures of antibodies (including scFvs and other molecules comprising, or alternatively consisting of, antibody fragments or variants thereof) that immunospecifically bind to BCL-6, wherein the mixture has at least one, two, three, four, five or more different antibodies described herein. In particular, the invention provides for mixtures of different antibodies that immunospecifically bind to the BCL-6. In some embodiments, the invention provides mixtures of at least 2, preferably at least 4, at least 6, at least 8, at least 10, at least 12, at least 15, at least 20, or at least 25 different antibodies that immunospecifically bind to BCL-6, wherein at least 1, at least 2, at least 4, at least 6, or at least 10, antibodies of the mixture is an antibody described herein. In some embodiments, each antibody of the mixture is an antibody described herein.

### Panels of Antibodies

The present invention also provides for panels of antibodies (including scFvs and other molecules comprising, or alternatively consisting of, antibody fragments or variants thereof) that immunospecifically bind to BCL-6, wherein the panel has at least one, two, three, four, five or more different antibodies described herein. In particular, the invention provides for panels of different antibodies that immunospecifically bind to the BCL-6. In some embodiments, the invention provides for panels of antibodies that have different affinities for BCL-6, different specificities for BCL-6, or different dissociation rates. The invention provides panels of at least 10, preferably at least 25, at least 50, at least 75, at least 100, at least 125, at least 150, at least 175, at least 200, at least 250, at least 300, at least 350, at least 400, at least 450, at least 500, at least 550, at least 600, at least 650, at least 700, at least 750, at least 800, at least 850, at least 900, at least 950, or at least 1000, antibodies. Panels of antibodies can be used, for example, in 96 well plates for assays such as ELISAs.

### Compositions

The present invention further provides for compositions comprising, one or more antibodies (including scFvs and other molecules comprising, or alternatively consisting of antibody fragments or variants described herein). In one embodiment, a composition of the present invention comprises, one, two, three, four, five, or more antibodies that comprise or alternatively consist of, a polypeptide having an amino acid sequence of any one or more of the heavy chain constant regions or a variant thereof. In another embodiment, a composition of the present invention comprises, one, two, three, four, five, or more antibodies that comprise, or alternatively consist of, a polypeptide having an amino acid sequence of any one or more of the light chain constant region or a variant thereof.

In some embodiments, a composition of the present invention comprises, one, two, three, four, five, or more antibodies that comprise, or alternative consist of, the heavy chain constant regions or a variant thereof and a light chain constant region or a variant thereof.

As discussed in more detail below, a composition described herein may be used either alone or in combination with other compositions. The antibodies (including scFvs and other molecules comprising, or alternatively consisting of antibody fragments or variants of the present invention) may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP Patent No. 0 396 387, each of which is incorporated herein by reference in its entirety.

The composition described herein may be a pharmaceutical composition. The composition, including pharmaceutical compositions, may comprise an antibody or antigen-binding fragment described herein and an adjuvant, carrier, buffers, antioxidants, wetting agents, lubricating agents, gelling agents, thickening agents, binding agents, disintegrating agents, humectants, preservatives, diluent, stabilizer, filler, excipient, or a combination thereof.

Antibodies described herein (including scFvs and other molecules comprising, or alternatively consisting of antibody fragments or variants of the present invention) may be used, for example, but not limited to, to purify and detect BCL-6, and to target the polypeptides of the present invention to cells expressing BCL-6, including both in vitro and in vivo diagnostic and therapeutic methods. For example, the antibodies have use in immunoassays for qualitatively and quantitatively measuring levels of BCL-6 in biological samples. See, e.g., Harlow et al. Antibodies: A Laboratory Manual, (Cold Spring Harbor Laboratory Press, 2nd ed. 1988) (incorporated herein by reference in its entirety).

### Nucleic Acids, Vectors, and Host Cells

The present invention also provides for a nucleic acid molecule encoding an antibody described herein (including molecules comprising, or alternatively consisting of, antibody fragments or variants thereof). The nucleic acid may be isolated, recombinant and/or engineered.

Nucleic acid molecules that encode the anti-BCL-6 antibodies described herein. The nucleic acids may be present in whole cells, in a cell lysate, or in a partially purified or substantially pure form. A nucleic acid may be isolated by purification away from other cellular components or other contaminants (e.g., other cellular nucleic acids or proteins) by standard techniques, including alkaline/SDS treatment, CsCl banding, column chromatography, agarose gel electrophoresis and others well known in the art. See Ausubel, et al. (2011) Current Protocols in Molecular Biology John Wiley & Sons, Inc., incorporated herein by reference in its entirety A nucleic acid described herein may be, for example, DNA or RNA and may or may not contain intronic sequences. The nucleic acid may be a cDNA molecule. Nucleic acids described herein may be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma may be obtained by standard PCR amplification or cDNA cloning techniques. For antibodies obtained from an immunoglobulin gene library (e.g., using phage display techniques), nucleic acid encoding the antibody may be recovered from the library. Specifically, degenerate codon substitutions may be achieved by generating, e.g., sequences in which the third position of one or more selected codons is substituted with mixed-base and/or deoxyinosine residues. Batzer, et al. (1991) Nucleic Acid Res. 19: 5081; Ohtsuka, et al. (1985) J. Biol. Chem. 260: 2605-08; Rossolini, et al. (1994) Mol. Cell. Probes 8: 91-98, each of which is incorporated herein by reference in its entirety.

As the codon(s) coding for each amino acid are known, nucleic acid sequences encoding the antibodies, fragments thereof, peptides, and/or proteins disclosed herein may be determined by those of skill in the art using manual or automated reverse translation, in which the amino acid sequence of the antibody, fragment thereof, peptide, and/or protein is reversed translated into the appropriate nucleic acid. Due to the degeneracy of the genetic code, more than one codon may encode a given amino acid. Cells from different organisms may exhibit preference(s) for different codons. Accordingly, in a process referred to as codon optimization, a nucleic acid may be optimized for expression in a given cell while not mutating the sequence coded for. Nucleic acids coding for antibodies or fragments thereof, proteins, and/or peptides disclosed herein may be codon optimized.

### Methods for Producing Antibodies

The antibodies described herein (including scFvs and other molecules comprising, or alternatively consisting of antibody fragments or variants described herein) can be produced by any method known in the art for the synthesis of antibodies, in particular, by chemical synthesis or preferably, by recombinant expression techniques.

Single chain Fvs (scFvs) that immunospecifically bind BCL-6 may be generated using phage display methods known in the art. In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of lymphoid tissues) or synthetic cDNA libraries. The DNA encoding the VH and VL domains are joined together by an scFv linker by PCR and cloned into a phagemid vector (e.g., p CANT AB 6 or pComb 3 HSS). The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to an antigen of interest (e.g., BCL-6 or a fragment or variant thereof) can be selected or identified with antigen, e.g., using labeled antigen or antigen bound or captured to a solid surface or bead. Examples of phage display methods that can be used to make the antibodies of the present invention include, but are not limited to, those disclosed in Brinkman et al. J. Immunol. Methods 182:41-50 (1995); Ames et al. J. Immunol. Methods 184:177-186 (1995); Kettleborough et al. Eur. J. Immunol. 24:952-958 (1994); Persic et al. Gene 187 9-18 (1997); Burton et al. Advances in Immunology 57:191-280(1994); PCT application No. PCT/GB91/01 134; PCT publications WO 90/02809; WO 91/10737; WO 92/01047; WO 92/18619; WO 93/11236; WO 95/15982; WO 95/20401; WO 97/13844; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human or humanized antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in PCT publication WO 92/22324; Mullinax et al. BioTechniques 12(6):864-869 (1992); Sawai et al. AJRI 34:26-34 (1995); and Better et al. Science 240:1041-1043 (1988), each of which is incorporated herein by reference in its entirety).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lambda constant regions. Preferably, the vectors for expressing the VH or VL domains comprise a promoter suitable to direct expression of the heavy and light chains in the chosen expression system, a secretion signal, a cloning site for the immunoglobulin variable domain, immunoglobulin constant domains, and a selection marker such as neomycin. The VH and VL domains may also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

Once an antibody molecule described herein (including molecules comprising, or alternatively consisting of, antibody fragments or variants thereof) has been chemically synthesized or recombinantly expressed, it may be purified by any method known in the art for purification of an immunoglobulin molecule, or more generally, a protein molecule, such as, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention may be fused to heterologous polypeptide sequences described herein or otherwise known in the art, to facilitate purification.

The present invention also provides methods for recombinantly producing the anti-BCL-6 antibodies described herein. Methods of producing the antibodies described herein are well known to those of ordinary skill in the art. The anti-BCL-6 antibodies described herein may also be produced by constructing, using conventional techniques well known to those of ordinary skill in the art, an expression vector containing an operon and a DNA sequence encoding the anti-BCL-6 antibodies described herein. Furthermore, the invention relates to vectors, especially plasmids, cosmids, viruses, bacteriophages and other vectors common in genetic engineering, which contain the above-mentioned nucleic acid molecules described herein. The nucleic acid molecules contained in the vectors may be linked to regulatory elements that ensure the transcription in prokaryotic and eukaryotic cells.

Vectors contain elements that facilitate manipulation for the expression of a foreign protein within the target host cell. Conveniently, manipulation of sequences and production of DNA for transformation is first performed in a bacterial host (e.g., E. coli) and usually vectors will include sequences to facilitate such manipulations, including a bacterial origin of replication and appropriate bacterial selection marker. Selection markers encode proteins necessary for the survival or growth of transformed host cells grown in a selective culture medium. Host cells not transformed with the vector containing the selection gene will not survive in the culture medium. Typical selection genes encode proteins that confer resistance to antibiotics or other toxins, complement auxotrophic deficiencies, or supply critical nutrients not available from complex media. Exemplary vectors and methods for transformation of yeast are described in the art. See, e.g., Burke, et al. (2000) Methods in Yeast Genetics Cold Spring Harbor Laboratory Press, incorporated herein by reference in its entirety.

The polynucleotide coding for the anti-BCL-6 antibodies may be operably linked to transcriptional and translational regulatory sequences that provide for expression of the polypeptide in yeast cells. These vector components may include, but are not limited to, one or more of the following: an enhancer element, a promoter, and a transcription termination sequence. Sequences for the secretion of the polypeptide may also be included (e.g., a signal sequence).

Nucleic acids are “operably linked” when placed into a functional relationship with another nucleic acid sequence. For example, DNA for a signal sequence is operably linked to DNA for a polypeptide if it is expressed as a preprotein that participates in the secretion of the polypeptide; a promoter or enhancer is operably linked to a coding sequence if it affects the transcription of the sequence. Generally, “operably linked” refers broadly to contiguous linked DNA sequences, and, in the case of a secretory leader, contiguous and in reading frame. However, enhancers do not have to be contiguous.

Promoters are untranslated sequences located upstream (5′) to the start codon of a structural gene (generally within about 100 to 1000 bp) that control the transcription and translation of particular nucleic acid sequences to which they are operably linked. Such promoters fall into several classes: inducible, constitutive, and repressible promoters (e.g., that increase levels of transcription in response to absence of a repressor). Inducible promoters may initiate increased levels of transcription from DNA under their control in response to some change in culture conditions (e.g., the presence or absence of a nutrient or a change in temperature.)

The expression vectors are transfected into a host cell by convention techniques well known to those of ordinary skill in the art to produce a transfected host cell, said transfected host cell cultured by conventional techniques well known to those of ordinary skill in the art to produce said anti-BCL-6 antibodies.

The host cells used to express the anti-BCL-6 antibodies may be either a bacterial cell such as E. coli, yeast (e.g., S. cerevisiae), or a eukaryotic cell (e.g., a mammalian cell line). A mammalian cell of a well-defined type for this purpose, such as a myeloma cell, 3T3, HeLa, C6A2780, Vero, MOCK II, a Chinese hamster ovary (CHO), Sf9, Sf21, COS, NSO, or HEK293 cell line may be used.

The general methods by which the vectors may be constructed, transfection methods required to produce the host cell and culturing methods required to produce the antibodies, and fragments thereof, from said host cells all include conventional techniques. Although preferably the cell line used to produce the anti-BCL-6 antibodies is a mammalian cell line, any other suitable cell line, such as a bacterial cell line such as an E. coli-derived bacterial strain, or a yeast cell line, may be used.

Similarly, once produced the anti-BCL-6 antibodies may be purified according to standard procedures in the art, such as for example cross-flow filtration, ammonium sulphate precipitation, and affinity column chromatography.

### Diagnostic Uses of Anti-BCL-6 Antibodies

Labeled antibodies described herein (including molecules comprising, or alternatively consisting of, antibody fragments or variants thereof) which specifically bind to BCL-6 can be used for diagnostic purposes to detect, diagnose, prognose, or monitor diseases and/or disorders associated with the angiogenesis. The invention provides for the detection of active angiogenesis, including pathological angiogenesis, comprising: (a) assaying the presence of BCL-6 in a biological sample from a subject using one or more antibodies described herein that immunospecifically binds to BCL-6; and (b) comparing the level of BCL-6 with a control, e.g., in normal biological samples, with no known pathological angiogenesis.

By “biological sample” is intended any fluids and/or cells obtained from a subject, body fluid, body tissue, body cell, cell line, tissue culture, or other source which may contain BCL-6 protein or mRNA. Body fluids include, but are not limited to, sera, plasma, urine, synovial fluid, spinal fluid, saliva, and mucous. Tissues samples may be taken from virtually any tissue in the body. Tissue samples may also be obtained from autopsy material. Methods for obtaining tissue biopsies and body fluids from mammals are well known in the art. Where the biological sample is to include mRNA, a tissue biopsy is the preferred source.

In addition, the anti-BCL-6 antibodies described herein are useful in diagnostic assays for BCL-6 expression in specific cells or tissues wherein the antibodies are labeled as described below and/or are immobilized on an insoluble matrix. Anti-BCL-6 antibodies also are useful for the affinity purification of BCL-6 from recombinant cell culture or natural sources.

Anti-BCL-6 antibodies can be used for the detection of BCL-6 in any one of a number of well-known diagnostic assay methods. For example, a biological sample may be assayed for BCL-6 by obtaining the sample from a desired source, admixing the sample with anti-BCL-6 antibody to allow the antibody to form antibody/BCL-6 complex with any BCL-6 present in the mixture, and detecting any antibody/BCL-6 complex present in the mixture. The biological sample may be prepared for assay by methods known in the art which are suitable for the particular sample. The methods of admixing the sample with antibodies and the methods of detecting antibody/BCL-6 complex are chosen according to the type of assay used. Such assays include competitive and sandwich assays, and steric inhibition assays. Competitive and sandwich methods employ a phase-separation step as an integral part of the method while steric inhibition assays are conducted in a single reaction mixture.

Analytical methods for BCL-6 all use one or more of the following reagents: labeled BCL-6 analogue, immobilized BCL-6 analogue, labeled anti-BCL-6 antibody, immobilized anti-BCL-6 antibody and steric conjugates. The labeled reagents also are known as “tracers.”

The label used is any detectable functionality that does not interfere with the binding of BCL-6 and anti-BCL-6 antibody. Numerous labels are known for use in immunoassay, examples including moieties that may be detected directly, such as fluorochrome, chemiluminescent, and radioactive labels, as well as moieties, such as enzymes, that must be reacted or derivatized to be detected. Examples of such labels include the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luciferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

Conventional methods are available to bind these labels covalently to proteins or polypeptides. For instance, coupling agents such as dialdehydes, carboduimides, dimaleimides, bis-imidates, bis-diazotized benzidine, and the like may be used to tag the antibodies with the above-described fluorescent, chemiluminescent, and enzyme labels. See, for example, U.S. Pat. No. 3,940,475 (fluorimetry) and 3,645,090 (enzymes); Hunter et al. Nature 144: 945 (1962); David et al. Biochemistry 13: 1014-1021 (1974); Pain et al. J. Immunol. Methods, 40: 219-230 (1981); and Nygren J. Histochem. and Cytochem. 30: 407-412 (1982), each of which is incorporated herein by reference in its entirety. Preferred labels herein include enzymes such as horseradish peroxidase and alkaline phosphatase.

The conjugation of such label, including the enzymes, to the antibody is a standard manipulative procedure for one of ordinary skill in immunoassay techniques. See, for example, O'Sullivan et al. “Methods for the Preparation of Enzyme-antibody Conjugates for Use in Enzyme Immunoassay,” in Methods in Enzymology, ed. J. J. Langone and H. Van Vunakis, Vol. 73 (Academic Press, New York, N.Y., 1981), incorporated herein by reference in its entirety, pages 147-166.

Immobilization of reagents is required for certain assay methods. Immobilization entails separating the anti-BCL-6 antibody from any BCL-6 that remains free in solution. This conventionally is accomplished by either insolubilizing the anti-BCL-6 antibody or BCL-6 analogue before the assay procedure, as by adsorption to a water-insoluble matrix or surface (U.S. Pat. No. 3,720,760), by covalent coupling (for example, using glutaraldehyde cross-linking), or by insolubilizing the anti-BCL-6 antibody or BCL-6 analogue afterward, e.g., by immunoprecipitation.

Other assay methods, known as competitive or sandwich assays, are well established and widely used in the commercial diagnostics industry.

Competitive assays rely on the ability of a tracer BCL-6 analogue to compete with the test sample BCL-6 for a limited number of anti-BCL-6 antibody antigen-binding sites. The anti-BCL-6 antibody generally is insolubilized before or after the competition and then the tracer and BCL-6 bound to the anti-BCL-6 antibody are separated from the unbound tracer and BCL-6. This separation is accomplished by decanting (where the binding partner was preinsolubilized) or by centrifuging (where the binding partner was precipitated after the competitive reaction). The amount of test sample BCL-6 is inversely proportional to the amount of bound tracer as measured by the amount of marker substance. Dose-response curves with known amounts of BCL-6 are prepared and compared with the test results to quantitatively determine the amount of BCL-6 present in the test sample. These assays are called ELISA systems when enzymes are used as the detectable markers.

Another species of competitive assay, called a “homogeneous” assay, does not require a phase separation. Here, a conjugate of an enzyme with the BCL-6 is prepared and used such that when anti-BCL-6 antibody binds to the BCL-6 the presence of the anti-BCL-6 antibody modifies the enzyme activity. In this case, the BCL-6 or its immunologically active fragments are conjugated with a bifunctional organic bridge to an enzyme such as peroxidase. Conjugates are selected for use with anti-BCL-6 antibody so that binding of the anti-BCL-6 antibody inhibits or potentiates the enzyme activity of the label. This method per se is widely practiced under the name of EMIT.

Steric conjugates are used in steric hindrance methods for homogeneous assay. These conjugates are synthesized by covalently linking a low-molecular-weight hapten to a small BCL-6 fragment so that antibody to hapten is substantially unable to bind the conjugate at the same time as anti-BCL-6 antibody. Under this assay procedure the BCL-6 present in the test sample will bind anti-BCL-6 antibody, thereby allowing anti-hapten to bind the conjugate, resulting in a change in the character of the conjugate hapten, e.g., a change in fluorescence when the hapten is a fluorophore.

Sandwich assays particularly are useful for the determination of BCL-6 or anti-BCL-6 antibodies. In sequential sandwich assays an immobilized anti-BCL-6 antibody is used to adsorb test sample BCL-6, the test sample is removed as by washing, the bound BCL-6 is used to adsorb a second, labeled anti-BCL-6 antibody and bound material is then separated from residual tracer. The amount of bound tracer is directly proportional to test sample BCL-6. In “simultaneous” sandwich assays the test sample is not separated before adding the labeled anti-BCL-6. A sequential sandwich assay using an anti-BCL-6 monoclonal antibody as one antibody and a polyclonal anti-BCL-6 antibody as the other is useful in testing samples for BCL-6.

The foregoing are merely exemplary diagnostic assays for BCL-6. Other methods now or hereafter developed that use anti-BCL-6 antibody for the determination of BCL-6 are included within the scope hereof, including the bioassays described above.

Antibodies described herein (including molecules comprising, or alternatively consisting of, antibody fragments or variants thereof) which specifically bind to BCL-6 can be used for diagnostic purposes to detect, diagnose, prognose, or monitor neovasculization of tumors and conditions associated therewith.

### Pharmaceutically Acceptable Salts

Another embodiment of the present invention relates to an antibody (including scFvs and other molecules comprising, or alternatively consisting of antibody fragments or variants of the present invention) described herein that has been synthetically produced (e.g., synthesized) as a pharmaceutically acceptable salt.

Another embodiment of the present invention relates to a peptide consists or consists essentially of an amino acid sequence according to SEQ ID NO: 1, 2, or a combination thereof, that has been synthetically produced (e.g., synthesized) as a pharmaceutically acceptable salt.

Methods to synthetically produce peptides are well known in the art. The salts of the peptides according to the present invention differ substantially from the peptides in their state(s) in vivo, as the peptides as generated in vivo are no salts. The non-natural salt form of the peptide mediates the solubility of the peptide, in particular in the context of pharmaceutical compositions comprising the peptides.

A sufficient and at least substantial solubility of the peptide(s) is required in order to efficiently provide the peptides to the subject to be treated. Preferably, the salts are pharmaceutically acceptable salts of the peptides. These salts according to the invention include alkaline and earth alkaline salts such as salts of the Hofmeister series comprising as anions PO₄³⁻, SO₄²⁻, CH₃COO⁻, Cl⁻, Br⁻, NO₃⁻, ClO₄⁻, I⁻, SCN⁻ and as cations NH₄⁺, Rb⁺, K⁺, Na⁺, Cs⁺, Li⁺, Zn²⁺, Mg²⁺, Ca²⁺, Mn²⁺, Cu²⁺ and Ba²⁺. Particularly salts are selected from (NH₄)₃PO₄, (NH₄)₂HPO₄, (NH₄)H₂PO₄, (NH₄)₂SO₄, NH₄CH₃COO, NH₄Cl, NH₄Br, NH₄NO₃, NH₄ClO₄, NH₄I, NH₄SCN, Rb₃PO₄, Rb₂HPO₄, RbH₂PO₄, Rb₂SO₄, Rb₄CH₃COO, Rb₄Cl, Rb₄Br, Rb₄NO₃, Rb₄ClO₄, Rb₄I, Rb₄SCN, K₃PO₄, K₂HPO₄, KH₂PO₄, K₂SO₄, KCH₃COO, KCl, KBr, KNO₃, KClO₄, KI, KSCN, Na₃PO₄, Na₂HPO₄, NaH₂PO₄, Na₂SO₄, NaCH₃COO, NaCl, NaBr, NaNO₃, NaClO₄, NaI, NaSCN, ZnCI₂ Cs₃PO₄, Cs₂HPO₄, CsH₂PO₄, Cs₂SO₄, CsCH₃COO, CsCl, CsBr, CsNO₃, CsClO₄, CsI, CsSCN, Li₃PO₄, Li₂HPO₄, LiH₂PO₄, Li₂SO₄, LiCH₃COO, LiCl, LiBr, LiNO₃, LiClO₄, LiI, LiSCN, Cu₂SO₄, Mg₃(PO₄)₂, Mg₂HPO₄, Mg(H₂PO₄)₂, Mg₂SO₄, Mg(CH₃COO)₂, MgCl₂, MgBr₂, Mg(NO₃)₂, Mg(ClO₄)₂, MgI₂, Mg(SCN)₂, MnCl₂, Ca₃(PO₄), Ca₂HPO₄, Ca(H₂PO₄)₂, CaSO₄, Ca(CH₃COO)₂, CaCl₂), CaBr₂, Ca(NO₃)₂, Ca(ClO₄)₂, CaI₂, Ca(SCN)₂, Ba₃(PO₄)₂, Ba₂HPO₄, Ba(H₂PO₄)₂, BaSO₄, Ba(CH₃COO)₂, BaCl₂, BaBr₂, Ba(NO₃)₂, Ba(ClO₄)₂, BaI₂, and Ba(SCN)₂. Particularly preferred are NH acetate, MgCl₂, KH₂PO₄, Na₂SO₄, KCl, NaCl, and CaCl₂, such as, for example, the chloride or acetate (trifluoroacetate) salts.

Generally, peptides and variants (at least those containing peptide linkages between amino acid residues) may be synthesized by the Fmoc-polyamide mode of solid-phase peptide synthesis as disclosed by Stawikowski and Fields, Curr Protoc Protein Sci. (2002), Chapter 18: Unit-18.1, incorporated herein by reference in its entirety, and by references as cited therein. Temporary N-amino group protection is afforded by the 9-fluorenylmethyloxycarbonyl (Fmoc) group. Repetitive cleavage of this highly base-labile protecting group is done using 20% piperidine in N, N-dimethylformamide. Side-chain functionalities may be protected as their butyl ethers (in the case of serine threonine and tyrosine), butyl esters (in the case of glutamic acid and aspartic acid), butyloxycarbonyl derivative (in the case of lysine and histidine), trityl derivative (in the case of cysteine) and 4-methoxy-2,3,6-trimethylbenzenesulphonyl derivative (in the case of arginine). Where glutamine or asparagine are C-terminal residues, use is made of the 4,4′-dimethoxybenzhydryl group for protection of the side chain amido functionalities. The solid-phase support is based on a polydimethyl-acrylamide polymer constituted from the three monomers dimethylacrylamide (backbone-monomer), bisacryloylethylene diamine (cross linker) and acryloylsarcosine methyl ester (functionalizing agent). The peptide-to-resin cleavable linked agent used is the acid-labile 4-hydroxymethyl-phenoxyacetic acid derivative. All amino acid derivatives are added as their preformed symmetrical anhydride derivatives with the exception of asparagine and glutamine, which are added using a reversed N, N-dicyclohexyl-carbodiimide/1hydroxybenzotriazole mediated coupling procedure. All coupling and deprotection reactions are monitored using ninhydrin, trinitrobenzene sulphonic acid or isotin test procedures. Upon completion of synthesis, peptides are cleaved from the resin support with concomitant removal of side-chain protecting groups by treatment with 95% trifluoroacetic acid containing a 50% scavenger mix. Scavengers commonly used include ethanedithiol, phenol, anisole and water, the exact choice depending on the constituent amino acids of the peptide being synthesized. Also a combination of solid phase and solution phase methodologies for the synthesis of peptides is possible (see, for example, Bruckdorfer, et al., Current Pharmaceutical Biotechnology (2004) 5(1):29-43, incorporated herein by reference in its entirety, and the references as cited therein).

Trifluoroacetic acid is removed by evaporation in vacuo, with subsequent trituration with diethyl ether affording the crude peptide. Any scavengers present are removed by a simple extraction procedure which on lyophilization of the aqueous phase affords the crude peptide free of scavengers. Reagents for peptide synthesis are generally available from, e.g., Calbiochem-Novabiochem (Nottingham, UK).

Purification may be performed by anyone, or a combination of, techniques such as re-crystallization, size exclusion chromatography, ion-exchange chromatography, hydrophobic interaction chromatography and (usually) reverse-phase high performance liquid chromatography using e.g. acetonitrile/water gradient separation.

Analysis of peptides may be carried out using thin layer chromatography, electrophoresis, in particular capillary electrophoresis, solid phase extraction (CSPE), reverse-phase high performance liquid chromatography, amino-acid analysis after acid hydrolysis and by fast atom bombardment (FAB) mass spectrometric analysis, as well as MALDI and ESI-Q-TOF mass spectrometric analysis.

A “pharmaceutical composition” is a composition suitable for administration to a human being in a medical setting. Preferably, a pharmaceutical composition is sterile and produced according to GMP guidelines.

The pharmaceutical compositions comprise the antibodies (including scFvs and other molecules comprising, or alternatively consisting of antibody fragments or variants of the present invention) and/or peptides described herein either in the free form or in the form of a pharmaceutically acceptable salt (see also above). As used herein, “a pharmaceutically acceptable salt” refers to a derivative of the disclosed peptides wherein the peptide is modified by making acid or base salts of the agent. For example, acid salts are prepared from the free base (typically wherein the neutral form of the drug has a neutral —NH2 group) involving reaction with a suitable acid. Suitable acids for preparing acid salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methane sulfonic acid, ethane sulfonic acid, p-toluenesulfonic acid, salicylic acid, and the like, as well as inorganic acids, e.g., hydrochloric acid, hydrobromic acid, sulfuric acid, nitric acid phosphoric acid and the like. Conversely, preparation of basic salts of acid moieties which may be present on a peptide are prepared using a pharmaceutically acceptable base such as sodium hydroxide, potassium hydroxide, ammonium hydroxide, calcium hydroxide, trimethylamine or the like.

### Adjuvants

A pharmaceutical composition according to the disclosure may contain one or more adjuvants. Adjuvants include substances that non-specifically enhance or potentiate the immune response (e.g., immune responses mediated by CD8-positive T cells and helper-T (TH) cells to an antigen, and would thus be considered useful in the medicament of the present disclosure. Exemplary suitable adjuvants may be selected from the group consisting of colony-stimulating factors, such as Granulocyte Macrophage Colony Stimulating Factor (GM-CSF, sargramostim), cyclophosphamide, imiquimod, resiquimod, and interferon-alpha. In a preferred embodiment, in a pharmaceutical composition according to the disclosure, the adjuvant is selected from the group consisting of colony-stimulating factors, such as Granulocyte Macrophage Colony Stimulating Factor (GM-CSF, sargramostim), cyclophosphamide, imiquimod and resiquimod. In a preferred embodiment of the pharmaceutical composition according to the disclosure, the adjuvant is cyclophosphamide, imiquimod or resiquimod. Even more preferred adjuvants are Montanide IMS 1312, Montanide ISA 206, Montanide ISA 50V, Montanide ISA-51, poly-lCLC (Hiltonol®) and anti-CD40 mAB, or combinations thereof.

Suitable adjuvants also include, but are not limited to, 1018 ISS, aluminum salts, AMPLIVAX®, AS15, BCG, CP-870,893, CpG7909, CyaA, dSLIM, flagellin or TLR5 ligands derived from flagellin, FLT3 ligand, GM-CSF, IC30, IC31, lmiquimod (ALDARA®), resiquimod, lmuFact IMP321, Interleukins as IL-2, IL-13, IL-21, Interferon-alpha or -beta, or pegylated derivatives thereof, IS Patch, ISS, ISCOMATRIX, ISCOMs, Juvlmmune®, LipoVac, MALP2, MF59, monophosphoryl lipid A, Montanide IMS 1312, Montanide ISA 206, Montanide ISA 50V, Montanide ISA-51, water-in-oil and oil-in-water emulsions, OK-432, OM-17 4, OM-197-MPEC, ONTAK, OspA, PepTel® vector system, poly(lactide co-glycolide) [PLG]-based and dextran microparticles, talactoferrin SRL 172, Virosomes and other Virus-like particles, YF-17D, VEGF trap, R848, beta-glucan, Pam3Cys, Aquila's QS21 stimulon, which is derived from saponin, mycobacterial extracts and synthetic bacterial cell wall mimics, and other proprietary adjuvants such as Ribi's Detox, Quil, or Superfos. Adjuvants such as Freund's or GM-CSF are preferred. Several immunological adjuvants (e.g., MF59) specific for dendritic cells and their preparation have been described previously (Allison and Krummel, 1995). Also, cytokines may be used. Several cytokines have been directly linked to influencing dendritic cell migration to lymphoid tissues (e.g., TNF-), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12, IL-15, IL-23, IL-7, IFN-alpha. IFN-beta) (Gabrilovich et al., 1996).

CpG immunostimulatory oligonucleotides have also been reported to enhance the effects of adjuvants in a vaccine setting. Without being bound by theory, CpG oligonucleotides act by activating the innate (non-adaptive) immune system via Toll-like receptors (TLR), mainly TLR9. CpG triggered TLR9 activation enhances antigen-specific humoral and cellular responses to a wide variety of antigens, including antibodies or protein antigens, live or killed viruses, dendritic cell vaccines, autologous cellular vaccines and polysaccharide conjugates in both prophylactic and therapeutic vaccines. More importantly it enhances dendritic cell maturation and differentiation, resulting in enhanced activation of TH1 cells and strong cytotoxic T-lymphocyte (CTL) generation, even in the absence of CD4 T cell help. The TH1 bias induced by TLR9 stimulation is maintained even in the presence of vaccine adjuvants such as alum or incomplete Freund's adjuvant (IFA) that normally promote a TH2 bias. CpG oligonucleotides show even greater adjuvant activity when formulated or co-administered with other adjuvants or in formulations such as microparticles, nanoparticles, lipid emulsions or similar formulations, which are especially necessary for inducing a strong response when the antigen is relatively weak. They also accelerate the immune response and enable the antigen doses to be reduced by approximately two orders of magnitude, with comparable antibody responses to the full-dose vaccine without CpG in some experiments (Krieg, 2006). U.S. Pat. No. 6,406,705 81 describes the combined use of CpG oligonucleotides, non-nucleic acid adjuvants and an antigen to induce an antigen-specific immune response. A CpG TLR9 antagonist is dSLIM (double Stem Loop lmmunomodulator) by Mologen (Berlin, Germany) which is a preferred component of the pharmaceutical composition of the present disclosure. Other TLR binding molecules such as RNA binding TLR 7, TLR 8 and/or TLR 9 may also be used.

Other examples for useful adjuvants include, but are not limited to chemically modified CpGs (e.g. CpR, ldera), dsRNA analogues such as Poly(1:C) and derivatives thereof (e.g. AmpliGen®, Hiltonol®, poly-(ICLC), poly(IC-R), poly(1:C12U), non-CpG bacterial DNA or RNA as well as immunoactive small molecules and antibodies such as cyclophosphamide, sunitinib, Bevacizumab®, celebrex, NCX-4016, sildenafil, tadalafil, vardenafil, sorafenib, temozolomide, temsirolimus, XL-999, CP-547632, pazopanib, VEGF Trap, ZD2171, AZD2171, anti-CTLA4, other antibodies targeting key structures of the immune system (e.g., anti-CD40, anti-TGFbeta, anti-TNFalpha receptor) and SC58175, which may act therapeutically and/or as an adjuvant. The amounts and concentrations of adjuvants and additives useful in the context of the present disclosure can readily be determined by the skilled artisan without undue experimentation.

Preferred adjuvants are anti-CD40, imiquimod, resiquimod, GM-CSF, cyclophosphamide, sunitinib, bevacizumab, interferon-alpha, CpG oligonucleotides and derivatives, poly-(1:C) and derivates, RNA, sildenafil, and particulate formulations with PLG or virosomes. In a preferred embodiment, the pharmaceutical composition according to the disclosure the adjuvant is selected from the group consisting of colony-stimulating factors, such as Granulocyte Macrophage Colony Stimulating Factor (GM-CSF, sargramostim), cyclophosphamide, imiquimod, resiquimod, and interferon-alpha.

In a preferred embodiment, the pharmaceutical composition according to the disclosure the adjuvant is selected from the group consisting of colony-stimulating factors, such as Granulocyte Macrophage Colony Stimulating Factor (GM-CSF, sargramostim), cyclophosphamide, imiquimod and resiquimod. In a preferred embodiment of the pharmaceutical composition according to the disclosure, the adjuvant is cyclophosphamide, imiquimod or resiquimod. Even more preferred adjuvants are Montanide IMS 1312, Montanide ISA 206, Montanide ISA 50V, Montanide ISA-51, poly-lCLC (Hiltonol®) and anti-CD40 mAB, or combinations thereof.

### Therapeutic Methods and Therapeutic Compositions

The anti-BCL-6 antibodies and antigen binding fragments thereof described herein may be used in methods for the inhibition of angiogenesis in a tissue, and thereby inhibiting events in the tissue, which depends upon the nature of the angiogenesis. Generally, the method comprises administering to the tissue a composition comprising an angiogenesis-inhibiting amount of the anti-BCL-6 antibodies and antigen binding fragments thereof described herein.

Angiogenesis includes a variety of processes involving neovascularization of a tissue including “sprouting”, asculogenesis, or vessel enlargement, all of which angiogenesis processes involve disruption of extracellular matrix collagen in blood vessels. With the exception of traumatic wound healing, corpus leuteum formation and embryogenesis, it is believed that the majority of angiogenesis processes are associated with disease processes and therefore the use of the anti-BCL-6 antibodies and antigen binding fragments thereof described herein is selective for the disease.

A variety of diseases in which angiogenesis is believed to be important, referred to as angiogenic diseases, including but not limited to, inflammatory disorders such as immune and non-immune inflammation, chronic articular rheumatism and psoriasis, disorders associated with inappropriate or inopportune invasion of vessels such as diabetic retinopathy, neovascular glaucoma, restenosis, capillary proliferation in atherosclerotic plaques and osteoporosis, and cancer associated disorders, such as solid tumors, solid tumor metastases, angiofibromas, retrolental fibroplasia, hemangiomas, Kaposi's sarcoma and the like cancers which require neovascularization to support tumor growth. Other suitable tumors include melanoma, carcinoma, sarcoma, fibrosarcoma, glioma and astrocytoma. Thus, methods, which inhibit angiogenesis in a diseased tissue, ameliorate symptoms of the disease and, depending upon the disease, can contribute to treatment of the disease.

As described herein, any of a variety of tissues, or organs comprised of organized tissues, can support angiogenesis in disease conditions including skin, muscle, gut, connective tissue, joints, bones and the like tissue in which blood vessels can invade upon angiogenic stimuli. Tissue, as used herein, also encompasses all bodily fluids, secretions, serum, blood, cerebrospinal fluid, plasma, urine, synovial fluid, vitreous humor, or a mixture thereof.

Therapeutic formulations of the anti-BCL-6 antibodies described herein are prepared for storage by mixing antibody having the desired degree of purity with optional physiologically acceptable carriers, excipients, or stabilizers (Remington: The Science and Practice of Pharmacy, 19th Edition, Alfonso, R., ed, Mack Publishing Co. (Easton, Pa.: 1995), incorporated herein by reference in its entirety), in the form of lyophilized cake or aqueous solutions. Examples of excipients include buffers, binding agents, blasting agents, diluents, flavors, and/or lubricants. An extensive listing of excipients that can be used in such a composition, can be, for example, taken from A. Kibbe, Handbook of Pharmaceutical Excipients (Kibbe, 2000), incorporated herein by reference in its entirety. Acceptable carriers, excipients or stabilizers are nontoxic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG). Exemplary formulations can be found in, for example, EP2112253, incorporated herein by reference in its entirety.

The anti-BCL-6 antibody to be used for in vivo administration must be sterile. This is readily accomplished by filtration through sterile filtration membranes, prior to or following lyophilization and reconstitution. The anti-BCL-6 antibody may be stored in lyophilized form or in solution.

Therapeutic anti-BCL-6 antibody compositions generally are placed into a container having a sterile access port, for example, an intravenous solution bag or vial having a stopper pierceable by a hypodermic injection needle.

The route of anti-BCL-6 antibody administration is in accord with known methods, e.g. injection or infusion by intravenous, intraperitoneal, intracerebral, subcutaneous, intramuscular, intraocular, inhaled, optionally intransal, intrapulmonary, intraarterial, intracerebrospinal, or intralesional routes, or by sustained release systems as noted below. The anti-BCL-6 antibody may be administered as an aerosol, via intranasal and/or intrapulmonary routes. Preferably the antibody is given systemically.

Suitable examples of sustained-release preparations include semipermeable polymer matrices in the form of shaped articles, e.g. films, or microcapsules. Sustained release matrices include polyesters, hydrogels, polylactides (U.S. Pat. No. 3,773,919, EP 58,481, each of which is incorporated herein by reference in its entirety), copolymers of L-glutamic acid and gamma ethyl-L-glutamate (Sidman et al. Biopolymers, 22: 547-556 (1983)), poly (2-hydroxyethyl-methacrylate) (Langer et al. J. Biomed. Mater. Res., 15: 167-277 (1981) and Langer, Chem. Tech., 12: 98-105 (1982), each of which is incorporated herein by reference in its entirety), ethylene vinyl acetate (Langer et al. supra) or poly-D-(−)-3-hydroxybutyric acid (EP 133,988). Sustained-release anti-BCL-6 antibody compositions also include liposomally entrapped antibody. Liposomes containing antibody are prepared by methods known per se: DE 3,218,121; Epstein et al. Proc. Natl. Acad. Sci. USA, 82: 3688-3692 (1985); Hwang et al. Proc. Natl. Acad. Sci. USA, 77: 4030-4034 (1980); EP 52,322; EP 36,676; EP 88,046; EP 143,949; EP 142,641; Japanese patent application 83-118008; U.S. Pat. Nos. 4,485,045 and 4,544,545; and EP 102,324, each of which is incorporated herein by reference in its entirety. Ordinarily the liposomes are of the small (about 200-800 Angstroms) unilamelar type in which the lipid content is greater than about 30 mol. % cholesterol, the selected proportion being adjusted for the optimal antibody therapy.

An “effective amount” of anti-BCL-6 antibody and antigen binding fragments thereof described herein can be employed therapeutically will depend, for example, upon the therapeutic objectives, the route of administration, the type of anti-BCL-6 antibody employed, and the condition of the patient. Accordingly, it will be necessary for the therapist to titer the dosage and modify the route of administration as required to obtain the optimal therapeutic effect. Typically, the clinician will administer the anti-BCL-6 antibody until a dosage is reached that achieves the desired effect. The progress of this therapy is easily monitored by conventional assays.

### Routes of Administration

The anti-BCL-6 antibody and antigen binding fragments thereof described herein can be administered parentally by injection or by gradual infusion over time. Although the tissue to be treated can typically be accessed in the body by systemic administration and therefore most often treated by intravenous administration of therapeutic compositions, other tissues and delivery means are contemplated where there is a likelihood that the tissue targeted contains the target molecule. Thus, anti-BCL-6 antibody and antigen binding fragments thereof described herein may be administered intravenously, intraperitoneally, intramuscularly, subcutaneously, intracaviatary, intravesically, transdermally, topically, intraocually, orally, intranasally, or a combination thereof.

Therapeutic compositions of the present invention contain a physiologically tolerable carrier together with a therapeutically effective amount of anti-BCL-6 antibody and antigen binding fragments thereof described herein, dissolved or dispersed therein as an active ingredient. In a preferred embodiment, the therapeutic composition is not immunogenic or has reduced immunogenicity when administered to a mammal or human patient for therapeutic purposes. A therapeutically effective amount is an amount of anti-BCL-6 antibody and antigen binding fragments thereof described herein sufficient to produce a measurable inhibition of angiogenesis in the tissue being treated, e. g., an angiogenesis-inhibiting amount. Inhibition of angiogenesis can be measured in situ by immunohistochemistry, or by other methods known to one skilled in the art.

The anti-BCL-6 antibodies and antibody compositions described herein, may be used to treat, prevent, ameliorate, diagnose or prognose proliferative diseases, including but not limited to cancer, optionally solid tumors, in mammals, preferably humans.

Therapeutic and pharmaceutical compositions comprising the anti-BCL-6 antibody and antigen binding fragments thereof described herein may be used to treat, prevent, ameliorate, diagnose or prognose, angiogenic diseases, including but not limited to, inflammatory disorders such as immune and non-immune inflammation, chronic articular rheumatism and psoriasis, disorders associated with inappropriate or inopportune invasion of vessels such as diabetic retinopathy, neovascular glaucoma, restenosis, capillary proliferation in atherosclerotic plaques and osteoporosis, and cancer associated disorders, such as solid tumors, solid tumor metastases, angiofibromas, retrolental fibroplasia, hemangiomas, Kaposi's sarcoma and the like cancers which require neovascularization to support tumor growth. Other suitable tumors include melanoma, carcinoma, sarcoma, fibrosarcoma, glioma and astrocytoma.

Therapeutic or pharmaceutical compositions comprising the anti-BCL-6 antibodies described herein may be administered to an animal to treat, prevent or ameliorate a pathological angiogenesis disease or a disease characterized by undesirable excessive neovascularization, in particular chosen in the group consisting of cancers with abnormal angiogenesis, ophthalmological diseases with abnormal angiogenesis, rheumatoid arthritis, psoriasis, angioma, endometriosis and Kaposi sarcoma associated with the extracellular presence of human B cell lymphoma, and combinations thereof.

As a general proposition, the initial pharmaceutically effective amount of the antibody administered parenterally will be in the range of about 0.1 to 50 mg/kg of patient body weight per day, with the typical initial range of antibody used being 0.3 to 20 mg/kg/day, more preferably 0.3 to 15 mg/kg/day. The desired dosage can be delivered by a single bolus administration, by multiple bolus administrations, or by continuous infusion administration of antibody, depending on the pattern of pharmacokinetic decay that the practitioner wishes to achieve.

### Kits

A pharmaceutical pack or kit may comprise one or more containers filled with one or more of the ingredients of the pharmaceutical compositions comprising the anti-BCL-6 antibody and antigen binding fragments thereof described herein. Optionally associated with such container(s) can be a notice in the form prescribed by a governmental agency regulating the manufacture, use or sale of pharmaceuticals or biological products, which notice reflects approval by the agency of manufacture, use or sale for human administration.

Kits that can be used in the methods described herein. A kit may comprise an antibody described herein, preferably a purified antibody, in one or more containers. In an alternative embodiment, a kit comprises an antibody fragment that immunospecifically binds to BCL-6. Kits may contain a substantially isolated BCL-6 polypeptide as a control.

Kits may further comprise a control antibody which does not react with BCL-6. In another specific embodiment, the kits of the present invention contain a means for detecting the binding of an antibody to BCL-6 (e.g., the antibody may be conjugated to a detectable substrate such as a fluorescent compound, an enzymatic substrate, a radioactive compound or a luminescent compound, or a second antibody which recognizes the first antibody may be conjugated to a detectable substrate). In specific embodiments, the kit may include a recombinantly produced or chemically synthesized BCL-6. The BCL-6 provided in the kit may also be attached to a solid support. In a more specific embodiment, the detecting means of the above-described kit includes a solid support to which BCL-6 is attached. Such a kit may also include a non-attached reporter-labeled anti-human antibody. In this embodiment, binding of the antibody to BCL-6 can be detected by binding of the said reporter-labeled antibody.

A diagnostic kit for use in screening a biological sample containing antigens of the polypeptide described herein. The diagnostic kit includes a substantially isolated antibody specifically immunoreactive with BCL-6, and means for detecting the binding of BCL-6 to the antibody. In one embodiment, the antibody is attached to a solid support. In a specific embodiment, the antibody may be a monoclonal antibody. The detecting means of the kit may include a second, labeled monoclonal antibody. Alternatively, or in addition, the detecting means may include a labeled, competing antigen.

In one diagnostic configuration, a biological sample is reacted with a solid phase reagent having a surface-bound BCL-6 obtained by the methods of the present invention. After BCL-6 binds to a specific antibody, the unbound serum components are removed by washing, reporter-labeled anti-human antibody is added, unbound anti-human antibody is removed by washing, and a reagent is reacted with reporter-labeled anti-human antibody to bind reporter to the reagent in proportion to the amount of bound anti-BCL-6 antibody on the solid support. Typically, the reporter is an enzyme which is detected by incubating the solid phase in the presence of a suitable fluorometric, luminescent or colorimetric substrate.

The solid surface reagent in the above assay is prepared by known techniques for attaching protein material to solid support material, such as polymeric beads, dip sticks, 96-well plate or filter material. These attachment methods generally include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates can be used in conjunction with biotinylated antigen(s).

Thus, the invention provides an assay system or kit for carrying out this diagnostic method. The kit generally includes a support with surface-bound recombinant BCL-6, and a reporter-labeled anti-human antibody for detecting surface-bound anti-BCL-6 antibody.

Further details described herein can be found in the following example, which further defines the scope described herein. All references cited throughout the specification, and the references cited therein, are hereby expressly incorporated by reference in their entirety.

### Example 1

### Exemplary Amino Acid Sequences

Exemplary amino acid sequences are set forth in Table C.

TABLE C
Exemplary Amino Acid Sequences

| SEQ | | |
|---|---|---|
| ID | | |
| NO | Description | Sequence |
| 3 | Heavy Chain | QSLEESGGDLVKPGASLTLTCTASGFSFNKDAVCWVRQAPGKG |
| | Variable | LEWIACIYAGSSGSTYYATWAKGRFTISETSSTTVTLRMTSLTA |
| | Region (VH) | ADTATYFCSRGTNVWGPGTLVTVSS |
| 4 | Light Chain | ADIAMTQTPSSVEAAVGGTVTIKCQASQTINSYLAWYQQKPGQ |
| | Variable | PPKLLIYRASTLASGAPSRFKGSGSGTQFTLTISDLECADAATYY |
| | Region (VL) | CQSYYYSSNTAYGNTFGGGTDVVVK |
| 5 | Heavy Chain | GFSFNKD |
| | CDR1 | |
| 6 | Heavy Chain | YAGSSGS |
| | CDR2 | |
| 7 | Heavy Chain | GTNV |
| | CDR3 | |
| 8 | Heavy Chain | QSLEESGGDLVKPGASLTLTCTAS |
| | FR1 | |
| 9 | Heavy Chain | AVCWVRQAPGKGLEWIACI |
| | FR2 | |
| 10 | Heavy Chain | TYYATWAKGRFTISETSSTTVTLRMTSLTAADTATYFCSR |
| | FR3 | |
| 11 | Heavy Chain | WGPGTLVTVSS |
| | FR4 | |
| 12 | Light Chain | QASQTINSYLA |
| | CDR1 | |
| 13 | Light Chain | RASTLAS |
| | CDR2 | |
| 14 | Light Chain | QSYYYSSNTAYGNT |
| | CDR3 | |
| 15 | Light Chain | DIAMTQTPSSVEAAVGGTVTIKC |
| | FR1 | |
| 16 | Light Chain | WYQQKPGQPPKLLIY |
| | FR2 | |
| 17 | Light Chain | GAPSRFKGSGSGTQFTLTISDLECADAATYYC |
| | FR3 | |
| 18 | Light Chain | FGGGTDVVVK |
| | FR4 | |
| 19 | Heavy Chain | GQPKAPSVFPLAPCCGDTPSSTVTLGCLVKGYLPEPVTVTWNS |
| | Constant | GTLTNGVRTFPSVRQSSGLYSLSSVVSVTSSSQPVTCNVAHPAT |
| | Region | NTKVDKTVAPSTCSKPTCPPPELLGGPSVFIFPPKPKDTLMISRTP |
| | | EVTCVVVDVSQDDPEVQFTWYINNEQVRTARPPLREQQFNSTI |
| | | RVVSTLPIAHQDWLRGKEFKCKVHNKALPAPIEKTISKARGQPL |
| | | EPKVYTMGPPREELSSRSVSLTCMINGFYPSDISVEWEKNGKAE |
| | | DNYKTTPAVLDSDGSYFLYSKLSVPTSEWQRGDVFTCSVMHEA |
| | | LHNHYTQKSISRSPGK |
| 20 | Light Chain | GDPVAPTVLIFPPAADQVATGTVTIVCVANKYFPDVTVTWEVD |
| | Constant | GTTQTTGIENSKTPQNSADCTYNLSSTLTLTSTQYNSHKEYTCK |
| | Region | VTQGTTSVVQSFNRGDC |
| 21 | Full Heavy | QSLEESGGDLVKPGASLTLTCTASGFSFNKDAVCWVRQAPGKG |
| | Chain | LEWIACIYAGSSGSTYYATWAKGRFTISETSSTTVTLRMTSLTA |
| | | ADTATYFCSRGTNVWGPGTLVTVSSGQPKAPSVFPLAPCCGDT |
| | | PSSTVTLGCLVKGYLPEPVTVTWNSGTLTNGVRTFPSVRQSSGL |
| | | YSLSSVVSVTSSSQPVTCNVAHPATNTKVDKTVAPSTCSKPTCP |
| | | PPELLGGPSVFIFPPKPKDTLMISRTPEVTCVVVDVSQDDPEVQF |
| | | TWYINNEQVRTARPPLREQQFNSTIRVVSTLPIAHQDWLRGKEF |
| | | KCKVHNKALPAPIEKTISKARGQPLEPKVYTMGPPREELSSRSV |
| | | SLTCMINGFYPSDISVEWEKNGKAEDNYKTTPAVLDSDGSYFL |
| | | YSKLSVPTSEWQRGDVFTCSVMHEALHNHYTQKSISRSPGK |
| 22 | Full Light | ADIAMTQTPSSVEAAVGGTVTIKCQASQTINSYLAWYQQKPGQ |
| | Chain | PPKLLIYRASTLASGAPSRFKGSGSGTQFTLTISDLECADAATYY |
| | | CQSYYYSSNTAYGNTFGGGTDVVVKGDPVAPTVLIFPPAADQV |
| | | ATGTVTIVCVANKYFPDVTVTWEVDGTTQTTGIENSKTPQNSA |
| | | DCTYNLSSTLTLTSTQYNSHKEYTCKVTQGTTSVVQSFNRGDC |
| 23 | Light Chain | ADIAMTQTPSSVEAAVGGTVTIKC |
| | FR1 | |

### Example 2

### Selection of Anti-Bcl-6 Monoclonal Antibody

Five antibodies from a rabbit recombinant monoclonal antibody library were assessed for anti-Bcl-6 activity. The one with the greatest anti-Bcl-6 neutralizing activity was chosen for sequencing and further assessment as set forth herein.

### Example 3

### Sequencing of Anti-Bcl-6 Monoclonal Antibody

Seven digestions of the selected antibody were prepared using four different enzymes (Pepsin, Trypsin, Chymotrypsin, Asp N). The digestions for the sample were processed with disulfide reduction, alkylation, and then enzyme digestion. Each digestion contains peptides from all immunoglobulin chains. Digestions were analyzed by LC-MS/MS using a Thermo-Fisher Orbitrap Fusion™ mass spectrometer. Peptides were characterized from LC-MS/MS data using de novo peptide sequencing and then assembled into antibody sequences.

LC-MS/MS data of multiple enzyme digestions were mapped to the assembled antibody sequences. In the heavy chain and light chain, 100% of amino acid residues are covered by at least 5 peptide scans, with significant supporting fragment ions.

Leucine (L) and Isoleucine (I) have the same residue mass. This poses a challenge for accurately determining the identity of the two amino acids. Leucine and Isoleucine were determined based on w-ions in the data when the WILD® option is included. Supplementary information regarding enzyme specificity at L and I in chymotrypsin and pepsin digestions is also provided, along with statistical residue distribution of L and I at each position. See Table D (light chain variable region) and Table E (heavy chain variable region). See also Table C and FIG. 2A and FIG. 2B.

TABLE D
Leucine and Isoleucine Determination for Light Chain Variable Region

| Residue | Confidence | w-ions | Supplementary Information |
|---|---|---|---|
| I@3 | Confirmed | Yes | ++ No observation of chymotrypsin peptides digested |
| | | | after I@3 |
| | | | ++ No observation of pepsin peptides digested before or |
| | | | after I@3 |
| | | | + Residue frequency: L@3 = 6%, I@3 = 44% |
| I@22 | Confirmed | Yes | Low quantity of chymotrypsin peptides digested after |
| | | | I@22 |
| | | | −− High quantity of pepsin peptides digested before or |
| | | | after I@22 |
| | | | + Residue frequency: L@22 = 22%, I@22 = 69% |
| I@30 | Confirmed | Yes | ++ No observation of chymotrypsin peptides digested |
| | | | after I@30 |
| | | | + Very low quantity of pepsin peptides digested before or |
| | | | after I@30 |
| | | | + Residue frequency: L@30 = 10%, I@30 = 27% |
| L@34 | Confirmed | Yes | −− No observation of chymotrypsin peptides digested after |
| | | | L@34 |
| | | | ++ High quantity of pepsin peptides digested after L@34 |
| | | | ++ Residue frequency: L@34 = 53%, I@34 = 4% |
| L@47 | Confirmed | Yes | Very low quantity of chymotrypsin peptides digested after |
| | | | L@47 |
| | | | ++ Residue frequency: L@47 = 74%, I@47 < 1% |
| L@48 | Confirmed | Yes | ++ High quantity of chymotrypsin peptides digested after |
| | | | L@48 |
| | | | ++ Residue frequency: L@48 = 75%, I@48 = 3% |
| L@49 | Confirmed | Yes | ++ No observation of chymotrypsin peptides digested |
| | | | after I@49 |
| | | | ++ Residue frequency: L@49 = 2%, I@49 = 88% |
| L@55 | Confirmed | Yes | ++ High quantity of chymotrypsin peptides digested after |
| | | | L@55 |
| | | | ++ High quantity of pepsin peptides digested before or |
| | | | after L@55 |
| | | | ++ Residue frequency: L@55 = 34%, I@55 < 1% |
| L@74 | Confirmed | Yes | + Decent quantity of chymotrypsin peptides digested after |
| | | | L@74 |
| | | | + Decent quantity of pepsin peptides digested before or |
| | | | after L@74 |
| | | | ++ Residue frequency: L@74 = 96%, I@74 < 1% |
| I@76 | Confirmed | Yes | ++ No observation of chymotrypsin peptides digested |
| | | | after I@76 |
| | | | − Decent quantity of pepsin peptides digested before or |
| | | | after I@76 |
| | | | ++ Residue frequency: L@76 < 1%, I@76 = 97% |
| L@79 | Confirmed | Yes | −− No observation of chymotrypsin peptides digested after |
| | | | L@79 |
| | | | ++ High quantity of pepsin peptides digested before or |
| | | | after L@79 |
| | | | ++ Residue frequency: L@79 = 57%, I@79 < 1% |

TABLE E
Leucine and Isoleucine Determination for Heavy Chain Variable Region

| Residue | Confidence | w-ions | Supplementary Information |
|---|---|---|---|
| L@3 | Confirmed | Yes | −− No observation of chymotrypsin peptides digested after |
| | | | L@3 |
| | | | ++ High quantity of pepsin peptides digested before or |
| | | | after L@3 |
| | | | ++ Residue frequency: L@3 = 95%, I@3 < 1% |
| L@10 | Confirmed | Yes | −− No observation of chymotrypsin peptides digested after |
| | | | L@10 |
| | | | Low quantity of pepsin peptides digested before or after |
| | | | L@10 |
| | | | ++ Residue frequency: L@ 10 = 69%, I@10 < 1% |
| L@17 | Confirmed | Yes | + Decent quantity of chymotrypsin peptides digested after |
| | | | L@17 |
| | | | ++ High quantity of pepsin peptides digested before or |
| | | | after L@17 |
| | | | ++ Residue frequency: L@17 = 64%, I@17 < 1% |
| L@19 | Confirmed | Yes | ++ High quantity of chymotrypsin peptides digested after |
| | | | L@19 |
| | | | ++ High quantity of pepsin peptides digested before or |
| | | | after L@19 |
| | | | + Residue frequency: L@19 = 74%, I@19 = 10% |
| L@44 | Confirmed | Yes | Low quantity of chymotrypsin peptides digested after |
| | | | L@44 |
| | | | Low quantity of pepsin peptides digested before L@44 |
| | | | ++ Residue frequency: L@44 = 96%, I@44 < 1% |
| I@47 | Confirmed | Yes | ++ No observation of chymotrypsin peptides digested |
| | | | after I@47 |
| | | | ++ No observation of pepsin peptides digested after I@47 |
| | | | + Residue frequency: L@47 = 10%, I@47 = 37% |
| I@50 | Confirmed | Yes | ++ No observation of chymotrypsin peptides digested |
| | | | after I@50 |
| | | | −− High quantity of pepsin peptides digested before I@50 |
| | | | ++ Residue frequency: L@50 = 1%, I@50 = 85% |
| I@70 | Confirmed | Yes | ++ No observation of chymotrypsin peptides digested |
| | | | after I@70 |
| | | | ++ No observation of pepsin peptides digested after I@70 |
| | | | + Residue frequency: L@70 = 19%, I@70 = 66% |
| L@80 | Confirmed | Yes | ++ High quantity of chymotrypsin peptides digested after |
| | | | L@80 |
| | | | ++ High quantity of pepsin peptides digested before or |
| | | | after L@80 |
| | | | ++ Residue frequency: L@80 = 69%, I@80 < 1% |
| L@85 | Confirmed | Yes | + Decent quantity of chymotrypsin peptides digested after |
| | | | L@85 |
| | | | + Decent quantity of pepsin peptides digested before or |
| | | | after L@85 |
| | | | ++ Residue frequency: L@85 = 80%, I@85 < 1% |
| L@107 | Confirmed | Yes | ++ High quantity of chymotrypsin peptides digested after |
| | | | L@107 |
| | | | ++ High quantity of pepsin peptides digested before or |
| | | | after L@107 |
| | | | ++ Residue frequency: L@107 = 49%, I@107 < 1% |

The following observations were also made:

A significantly high amount of BSA was present in the sample. The sample was purified with Protein A beads and the enzyme digests were performed using an in-gel approach.

The N@ 179, within the constant region of the light chain, is at a consensus sequence for potential N-linked glycosylation.

O-linked glycosylation was detected on the heavy constant region approximately between positions 176 and 227.

N-linked glycosylation was detected on the heavy constant region at N@285.

Pyro-Glu(Q) modification was observed at N-terminus of the heavy chain.

Significant loss of C-terminal was lysine observed on the heavy chain.

### Example 4

### Anti-Bcl-6 Monoclonal Antibody Detects and Bind to Bcl-6

Total protein from human cells was extracted using a RIPA buffer with Halt Protease Inhibitor Cocktail (Thermo Fisher Scientific, Rockfold, IL). Twenty micrograms of total protein was electrophoresed onto Mini-PROTEAN TGX gels (Bio-Rad Laboratories, Hercules, CA) at 250 V for 20 min. The gels were subjected to transfer onto PVDF membranes using semidry transfer apparatus (Trans-Blot Turbo Transfer System; Bio-Rad Laboratories) at 25 V for 3 min. After blocking in Tris-buffered saline containing 3% I-Block (Thermo Scientific), the membrane was incubated overnight with primary antibodies at 4° C. followed by 1 hour incubation in horseradish peroxidase (HRP)-conjugated secondary antibody. The bound secondary antibody was detected using enhanced chemiluminescence. By this methodology, Bcl-6 was identified in human cells.

Lysates of human endothelial cells (HUVEC) were subjected to Western blot analysis for the presence of Bcl-6. Lysate subjected to primary and secondary antibody showed reactivity, while lysate with only secondary antibody was void of any reactivity. Primary Antibody was provided at 1/100 and secondary antibody was provided at 1/10,000. Only the combination of primary (anti-BCL-6 antibody) and secondary antibody showed any reactivity.

Lysates of a human endothelial cells (HUVEC and HDMEC) and human bladder cancer cell lines (T24, 5637 and 253J-BV) [20 μg of sample each] were subjected to Western blot analysis for the presence of Bcl-6. 50 ng of recombinant BCL6 protein was used as a positive control. Lysate subjected to primary and secondary antibody showed reactivity, while lysate with only secondary antibody was void of any reactivity.

Subcellular fractions from human bladder cancer cell line T24 were obtained from cytoplasm (Cyto), cell membrane (Mem), nucleus (Nuc) and compared to whole cellular lysate for the presence of Bcl-6. Whole lysate and fractionations lysates were subjected to Western blot analysis for the presence of Bcl-6. Bcl-6 was noted to be in the nucleus and cellular membrane. Lamin A, tubulin, and cadherin are markers for nucleus, cytoplasm, and membrane, respectively.

This data shows that the anti-BLC-6 antibodies described herein (IgG antibody having two heavy chains each comprising SEQ ID NO: 21 and two light chains each comprising SEQ ID NO: 22) selectively bind to BLC-6.

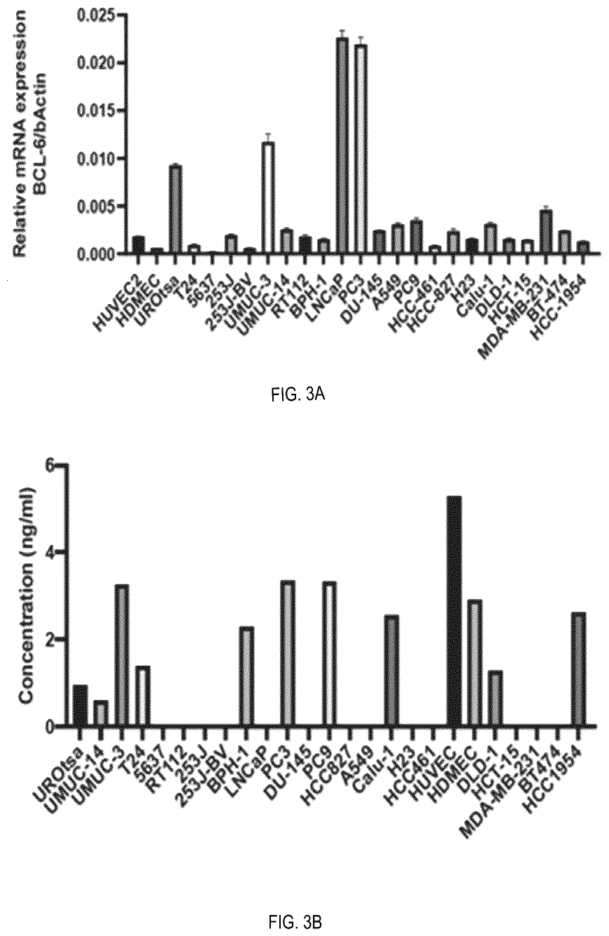

FIG. 3 depicts lysates from human cell lines and cell media supernatants subjected to RNA extraction and qPCR (FIG. 3A) or ELISA assay (FIG. 3B), respectively, for the presence of Bcl-6. Lysates of a panel of human cell lines (human endothelial cells, bladder cells, prostate cell, lung cells, colon cells and breast cells) were subjected to quantitative PCR using primers towards Bcl-6. Bcl-6 levels were highest in prostate (LNCaP and PC-3) and bladder (UROtsa and UMUC-3). Supernatants from these same cell lines were analyzed by ELISA assay towards Bcl-6. Secreted Bcl-6 was noted in all cell types (endothelial cells, bladder cells, prostate cell, lung cells, colon cells and breast cells) with the highest seen associated with endothelial cells. See FIG. 3B.

### Example 5

### The Anti-Bcl-6 Antibody can Inhibit Endothelial Cell Tube Formation

To assess the tube-forming ability of HUVEC and HDMEC cells, growth factor-reduced Matrigel (BD Bioscience, Bedford, MA) was allowed to solidify in the wells of a 48-well plate at 37° C. for 30 min. Next, HUVEC and HDMEC cells were seeded at a density of 3.0×10⁴ cells per well. For capillary tube formation experiments, endothelial cells were resuspended in EBM2 media with 20 μg/mL of control IgG or 20 μg/mL anti-Bcl-6 neutralizing antibody described herein (IgG antibody having two heavy chains each comprising SEQ ID NO: 21 and two light chains each comprising SEQ ID NO: 22) before seeding on Matrigel. Images of capillary tube formation were captured using Leica DMTL inverted microscopy (Leica Microsystems AG, Wetzlar, Germany) at 6 hours after seeding.

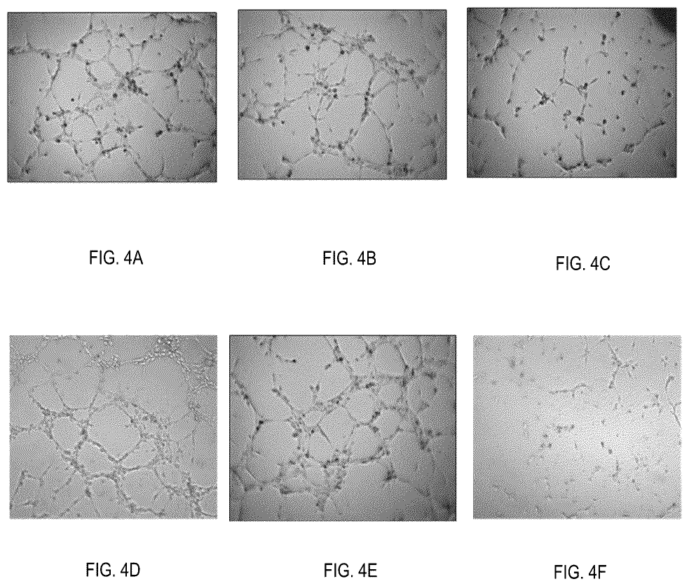

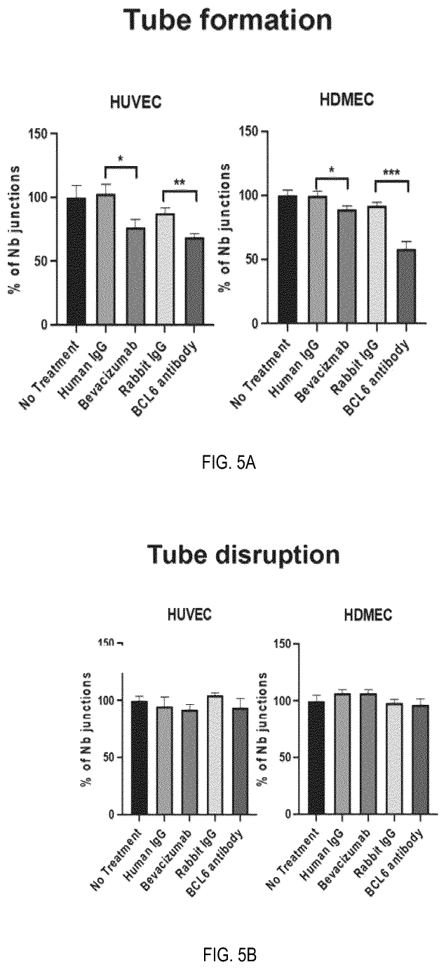

For capillary tube disruption experiments, cells were resuspended in EBM2 media without drugs and seeded and allowed to grow on Matigel for 6 hours prior to the initiation of drug treatment. Two hours after the addition of 20 μg/mL of control IgG or 20 μg/mL of anti-Bcl-6 antibody, images of vessels were captured. The antiangiogenic and vascular-disrupting activities of the antibodies were quantitatively evaluated by measuring the total tube length and surface area of capillary tubes, respectively, in at least 8 viewed fields. In both experiments, cells were constantly maintained at 37° C. and 5% CO₂. By this methodology, a monoclonal antibody described herein specific for Bcl-6 inhibits tube formation in both HMDEC and HUVEC cells. Data shown in FIG. 4 and FIG. 5.

FIG. 4 depicts the ability of the neutralizing anti-Bcl-6 antibody ability described herein to inhibit the ability of endothelial cells, human dermal microvascular endothelial cells (HDMEC) [FIG. 4A, FIG. 4B, FIG. 4C] and human umbilical vein endothelial cells (HUVEC) [FIG. 4D, FIG. 4E, FIG. 4F] to form capillaries. Neutralizing Bcl-6 antibody described herein (IgG antibody having two heavy chains each comprising SEQ ID NO: 21 and two light chains each comprising SEQ ID NO: 22) inhibited the ability of endothelial cells HDMEC and HUVEC to form capillaries. IgG serves as a negative control and AVASTIN® (bevacizumab) as a positive control. (A) HDMEC, no treatment; (B) HDMEC, treated with 0.7 μg/mL normal rabbit IgG; and (C) HDMEC treated with 0.7 mg/mL anti-BCL-6. (D) HUVEC, no treatment; (E) HUVEC, treated with 0.7 μg/mL normal rabbit IgG; and (C) HUVEC treated with 0.7 mg/mL anti-BCL-6. Each is a representative photograph from 96 well tube formation assay.

FIG. 5 depicts a graphical representation of tube formation assay (FIG. 5A) and tube disruption (FIG. 5B) for human dermal microvascular endothelial cells (HDMEC) and human umbilical vein endothelial cells (HUVEC) for no treatment, human IgG, AVASTIN® (bevacizumab, FDA approved drug, comparative control), rabbit IgG, or anti-BCL-6 antibody described herein (IgG antibody having two heavy chains each comprising SEQ ID NO: 21 and two light chains each comprising SEQ ID NO: 22). Both bevacizumab and the anti-Bcl-6 antibody described herein inhibited tube formation (which is generation of new blood vessels), while they have no effect on tube disruption (breakdown of existing blood vessels).

### Example 6

### Anti-Bcl-6 Antibody can Completely Block Huvec Proliferation

In order to demonstrate anti-angiogenic effects of the anti-Bcl-6 antibody described herein, the antibody was evaluated in a vascular endothelial cell proliferation assay.

Human umbilical vein endothelial cell proliferation assay is one of the most widely used in vitro model in angiogenesis research. HUVEC cells express Bcl-6. Therefore, HUVEC cells can be used to evaluate neutralization activity of the anti-Bcl-6 antibody.

In this assay, HUVEC cells were seeded into 24-well plates. After 24 hours, cells were fed with basal medium supplemented with 10 ng/ml Bcl-6. Bcl-6 antibody described herein (IgG antibody having two heavy chains each comprising SEQ ID NO: 21 and two light chains each comprising SEQ ID NO: 22) was added to wells at various concentrations (0.1-5 g/ml) and the plates were cultured at 37° C. in a 10% CO₂ incubator. After 24 hours, cells were digested by trypsin and cell numbers were counted. In control wells, normal IgG at various concentrations were added. Results showed that in the wells that contain 1 μg/ml of anti-Bcl-6 antibody, HUVEC proliferation was completely inhibited. In contrast, in the control wells in which the same amount (1 μg/ml) of normal antibody was added, no inhibitory effect on HUVEC proliferation was observed.

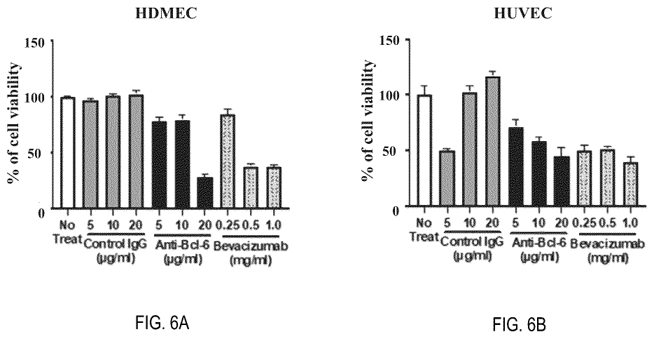

The anti-Bcl-6 antibody described herein can completely inhibit vascular endothelial cell proliferation and is capable of blocking angiogenesis (See, FIG. 6A and FIG. 6B). FIG. 6 depicts those neutralizing antibodies to Bcl-6 described herein reduced (IgG antibody having two heavy chains each comprising SEQ ID NO: 21 and two light chains each comprising SEQ ID NO: 22) cellular viability of HUVEC and HDMEC cells. Recombinant Bcl-6 was added to human endothelial cells and cell viability monitored. The addition of recombinant Bcl-6 did not increase the viability or proliferative ability) of these cells. Alternatively, neutralizing antibody to Bcl-6 reduced cellular viability of both HDMEC and HUVEC cells like what is seen with bevacizumab (FDA approved drug, comparative control), a neutralizing monoclonal antibody towards vascular endothelial growth factor (VEGF). Specifically, about 50-times lower amount of neutralizing antibody to Bcl-6 demonstrated the same efficacy as bevacizumab.

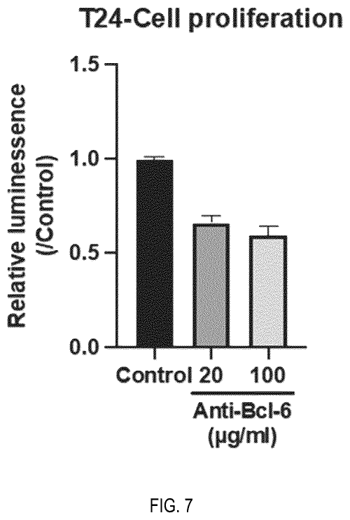

FIG. 7 depicts data showing that the neutralizing antibodies to Bcl-6 described herein (IgG antibody having two heavy chains each comprising SEQ ID NO: 21 and two light chains each comprising SEQ ID NO: 22) reduced cellular number of human bladder cancer cell line T24 after 72 hours of incubation, suggesting that the neutralizing antibodies to Bcl-6 inhibited cancer cellular proliferation.

### Example 7

Bcl-6 in the Urine of Bladder Cancer Patients is Greater than that in the Urine of Control Subjects

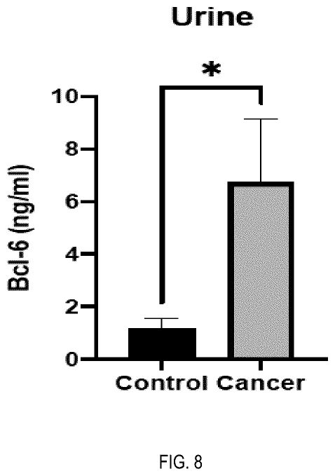

Voided urine samples from bladder cancer patients and controls (non-cancer) were analyzed with a commercial ELISA kit towards human Bcl-6. FIG. 8 depicts data showing that patients with bladder cancer (right bar) had more Bcl-6 in the urine (in ng/ml) than control subjects (left bar).

### Example 8

### Anti-Bcl-6 Antibody can Reduce Cellular Proliferation

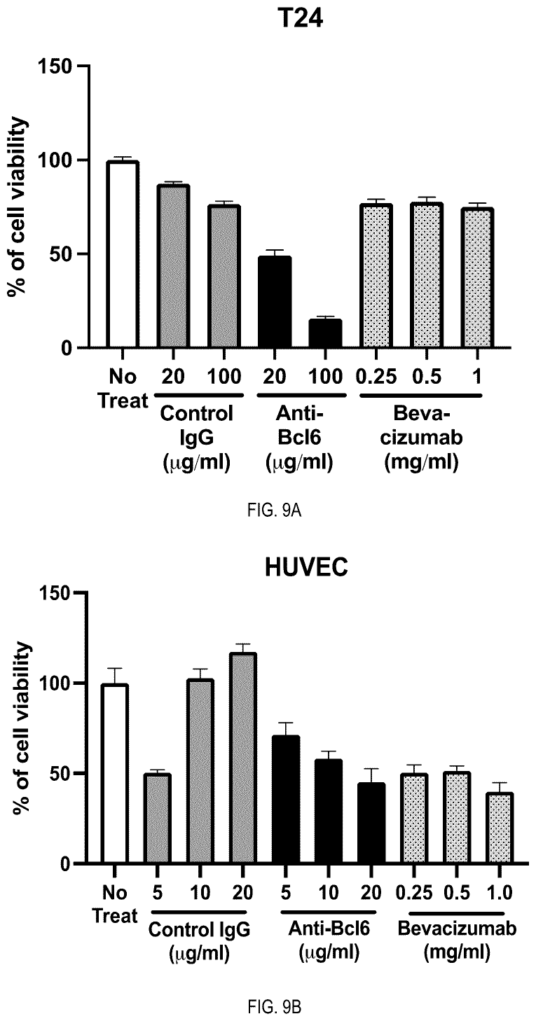

Cellular proliferation of T24 parental cells was measured by MTT assay at 72 hrs after treatment with no treatment, with increasing concentrations of control IgG (20 μg/ml or 100 μg/ml), with increasing concentrations of anti-Bcl-6 antibody disclosed herein (IgG antibody having two heavy chains each comprising SEQ ID NO: 21 and two light chains each comprising SEQ ID NO: 22) (20 μg/ml or 100 μg/ml), or with increasing concentrations of bevacizumab (0.25 mg/ml, 0.5 mg/ml, or 1 mg/ml). In a dose dependent manner, anti-Bcl-6 antibody disclosed herein reduced cellular proliferation, as shown in FIG. 9A, in which % of cell viability is plotted on the Y-axis. Capillary tube formation was measured in HUVECs cultured on growth factor-reduced Matrigel with no treatment, in the presence of increasing concentrations of control IgG (5 μg/ml, 10 μg/ml, or 20 μg/ml), in the presence of increasing concentrations of anti-Bcl-6 antibody disclosed herein (IgG antibody having two heavy chains each comprising SEQ ID NO: 21 and two light chains each comprising SEQ ID NO: 22) (5 μg/ml, 10 μg/ml, or 20 μg/ml), or in the presence of increasing concentrations of bevacizumab (0.25 mg/ml, 0.5 mg/ml, or 1 mg/ml). FIG. 9B, in which % of cell viability is plotted on the Y-axis, shows that capillary tube formation of HUVECs cultured in the presence of anti-Bcl-6 antibody disclosed herein (0, 20, and 100 μg/mL) demonstrated a reduction cellular viability similar to that of bevacizumab at 1 mg/ml. See FIGS. 9A and 9B.

### Example 9

### Anti-Bcl-6 can Antibody Reduce Tumor Growth

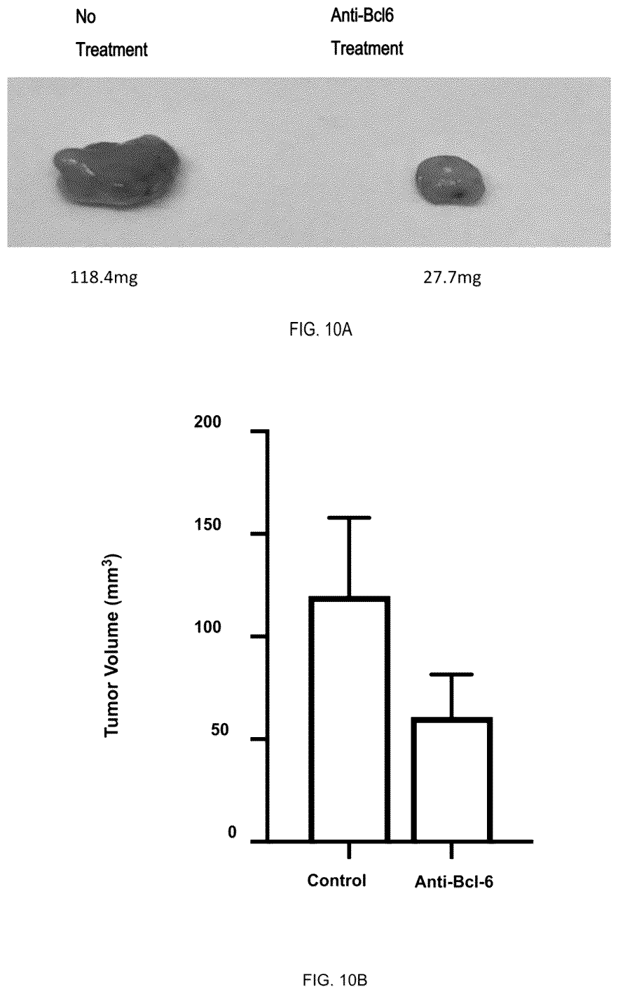

Tumor growth was established by subcutaneous injection of parental T24 cells into athymic mice (nu/nu). Anti-Bcl-6 antibody disclosed herein (IgG antibody having two heavy chains each comprising SEQ ID NO: 21 and two light chains each comprising SEQ ID NO: 22), a neutralizing monoclonal antibody to Bcl-6, was injected intratumorally 15 mcg twice weekly. Sterile PBS injection served as control. FIG. 10A and FIG. 10B are a photograph (FIG. 10A) and a graph (FIG. 10B) showing that tumors treated with anti-Bcl-6 antibody disclosed herein (right photograph and right bar) were significantly smaller than control tumors (left photograph and left bar). FIG. 10A shows that the control tumor had a weight of 118.4 mg, while the tumor treated with anti-Bcl-6 antibody disclosed herein had a weight of 27.7 mg. FIG. 10B shows tumor volume in mm³ plotted on the Y-axis, P=0.0166.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

Unless otherwise specified herein, ranges of values set forth herein are intended to operate as a scheme for referring to each separate value falling within the range individually, including but not limited to the endpoints of the ranges, and each separate value of each range set forth herein is hereby incorporated into the specification as if it were individually recited.

This specification may include references to “one embodiment”, “an embodiment”, “embodiments”, “one aspect”, “an aspect”, or “aspects”. Each of these words and phrases is not intended to convey a different meaning from the other words and phrases. These words and phrases may refer to the same embodiment or aspect, may refer to different embodiments or aspects, and may refer to more than one embodiment or aspect. Various embodiments and aspects may be combined in any manner consistent with this disclosure.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

1. A recombinant and/or isolated antibody or antigen binding fragment thereof comprising:
(a) a heavy chain variable domain comprising
(i) a complementarity-determining region (CDR) 1 comprising the amino acid sequence of SEQ ID NO: 5 comprising at most one conservative substitution,
(ii) a CDR 2 comprising the amino acid sequence of SEQ ID NO: 6 comprising at most one conservative substitution, and
(iii) a CDR 3 comprising the amino acid sequence of SEQ ID NO: 7 comprising at most one conservative substitution; and
(b) a light chain variable domain comprising
(i) a CDR 1 comprising the amino acid sequence of SEQ ID NO: 12 comprising at most one conservative substitution,
(ii) a CDR 2 comprising the amino acid sequence of SEQ ID NO: 13 comprising at most one conservative substitution, and
(iii) a CDR 3 comprising the amino acid sequence of SEQ ID NO: 14 comprising at most one conservative substitution.

2. The recombinant and/or isolated antibody or antigen binding fragment thereof of claim 1, comprising:
(a) a heavy chain variable domain comprising
(i) a CDR 1 comprising the amino acid sequence of SEQ ID NO: 5,
(ii) a CDR 2 comprising the amino acid sequence of SEQ ID NO: 6, and
(iii) a CDR 3 comprising the amino acid sequence of SEQ ID NO: 7; and
(b) a light chain variable domain comprising
(i) a CDR 1 comprising the amino acid sequence of SEQ ID NO: 12,
(ii) a CDR 2 comprising the amino acid sequence of SEQ ID NO: 13, and
(iii) a CDR 3 comprising the amino acid sequence of SEQ ID NO: 14.

3. The recombinant and/or isolated antibody or an antigen binding fragment thereof of claim 1,
(a) wherein the heavy chain variable domain comprises (i) a framework region (FR) 1 comprising an amino acid sequence at least about 95% identical to SEQ ID NO: 8, (ii) a FR 2 comprising an amino acid sequence at least about 95% identical to SEQ ID NO: 9, (iii) a FR 3 comprising an amino acid sequence at least about 95% identical to SEQ ID NO: 10, (iv) a FR 4 comprising an amino acid sequence at least about 95% identical to SEQ ID NO: 11; and
(b) wherein the light chain variable domain comprises (i) a FR 1 comprising an amino acid sequence at least about 95% identical to SEQ ID NO: 15, or a FR 1 comprising an amino acid sequence at least about 95% identical to SEQ ID NO: 23, (ii) a FR 2 comprising an amino acid sequence at least about 95% identical to SEQ ID NO: 16, (iii) a FR 3 comprising an amino acid sequence at least about 95% identical to SEQ ID NO: 17, (iv) a FR 4 comprising an amino acid sequence at least about 95% identical to SEQ ID NO: 18.

4-10. (canceled)

11. The recombinant antibody or antigen binding fragment thereof of claim 1, wherein the antibody or antigen binding fragment thereof is monoclonal.

12. The recombinant antibody or antigen binding fragment thereof of claim 1, wherein the antibody or antigen binding fragment thereof is humanized.

13. The recombinant antibody or antigen binding fragment thereof of claim 1, wherein the antibody or antigen binding fragment thereof is chimeric.

14-24. (canceled)

25. The recombinant antibody of claim 1, wherein the antibody or isolated antibody or antigen binding fragment thereof is conjugated to a detectable label.

26. The recombinant antibody or antigen binding fragment thereof of claim 25, wherein the antibody or isolated antibody or antigen binding fragment thereof is conjugated to a detectable label selected from a fluorescent label, luminescent label, bioluminescent label, radioactive label, chemiluminescent label, colorimetric label, fluorogenic label, enzymatic label, or a combination thereof.

27-46. (canceled)

47. A method for detecting a human B cell lymphoma 6 protein polypeptide comprising contacting a sample with the recombinant antibody of claim 1.

48. The method of claim 47, wherein the antibody or antigen binding fragment thereof, is attached to a solid phase support.

49. The method of claim 48, wherein the antibody is attached to a solid phase support.

50-51. (canceled)

52. A method for treating an angiogenesis-dependent condition comprising administering an effective amount of the recombinant antibody of claim 1.

53-56. (canceled)

57. The method of claim 52, wherein the condition is acute respiratory failure, retinopathy, age related macular degeneration, chronic articular rheumatism and psoriasis, disorders associated with inappropriate or inopportune invasion of vessels such as diabetic retinopathy, neovascular glaucoma, restenosis, capillary proliferation in atherosclerotic plaques and osteoporosis, and cancer associated disorders, such as solid tumors, solid tumor metastases, angiofibromas, retrolental fibroplasia, hemangiomas, Kaposi's sarcoma, cancer, optionally, solid tumors.

58. The method of claim 57, wherein the condition is cancer.

59. The method of claim 57, wherein the cancer comprises a solid tumor, optionally a tumor that requires neovascularization to support tumor growth.

60-164. (canceled)
